SEQUENCE LISTING (52 sequences)

SEQ ID NO: 1 - protein, 107 aa, Artificial Sequence [sequence omitted]

SEQ ID NO: 2 - protein, 107 aa, Artificial Sequence
  VL2
DIQMTQSPSSLSASVGDRVTITCRASQDVNIAVAWYQQKPGKAPKLLIYSASFLYSGVPSRFSGSRSGTDFTLTISSLQPEDFATYYCQQHYTTPPTFGQGTKVEIK

SEQ ID NO: 3 - protein, 107 aa, Artificial Sequence
  VL3
DIQMTQSPSSLSASVGDRVTITCRASQDVNTAVAWYQQKPGKAPKLLIYSASFLYSGVPSRFSGSRSGTDFTLTISSLQPEDFATYYCQQHYTYPPTFGQGTKVEIK

SEQ ID NO: 4 - protein, 107 aa, Artificial Sequence
  VL4
DIQMTQSPSSLSASVGDRVTITCRASQDVNIAVAWYQQKPGKAPKLLIYSASFLYSGVPSRFSGSRSGTDFTLTISSLQPEDFATYYCQQHYTYPPTFGQGTKVEIK

SEQ ID NO: 5 - protein, 107 aa, Artificial Sequence [sequence omitted]

SEQ ID NO: 6 - protein, 107 aa, Artificial Sequence
  VL6
DIQMTQSPSSLSASVGDRVTITCKASQDVSIGVAWYQQKPGKAPKLLIYSASYRYTGVPSRFSGSGSGTDFTLTISSLQPEDFATYYCQQYYITPYTFGQGTKVEIK

SEQ ID NO: 7 - protein, 214 aa, Artificial Sequence
  LC1
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 214 aa, Artificial Sequence
  LC2
DIQMTQSPSSLSASVGDRVTITCRASQDVNIAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 214 aa, Artificial Sequence
  LC3
DIQMTQSPSSLSASVGDRVTITCRASQDVNTAVAWYQQKPGKAPKLLIYSASFLYSGVPSRFSGSRSGTDFTLTISSLQPEDFATYYCQQHYTYPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 214 aa, Artificial Sequence
  LC4
DIQMTQSPSSLSASVGDRVTITCRASQDVNIAVAWYQQKPGKAPKLLIYSASFLYSGVPSRFSGSRSGTDFTLTISSLQPEDFATYYCQQHYTYPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 214 aa, Artificial Sequence
  LC5
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 214 aa, Artificial Sequence
  LC6
DIQMTQSPSSLSASVGDRVTITCKASQDVSIGVAWYQQKPGKAPKLLIYSASYRYTGVPSRFSGSGSGTDFTLTISSLQPEDFATYYCQQYYITPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 119 aa, Artificial Sequence [sequence omitted]

SEQ ID NO: 14 - protein, 120 aa, Artificial Sequence [sequence omitted]

SEQ ID NO: 15 - protein, 449 aa, Artificial Sequence
  HC1
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 16 - protein, 450 aa, Artificial Sequence
  HC2
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 17 - protein, 450 aa, Artificial Sequence
  HC3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 449 aa, Artificial Sequence
  HC4
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 19 - protein, 330 aa, Artificial Sequence
  FC1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 20 - protein, 330 aa, Artificial Sequence
  FC2
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 21 - protein, 471 aa, Artificial Sequence
  FC3
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 227 aa, Artificial Sequence
  FC4
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 23 - protein, 227 aa, Artificial Sequence
  FC5
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 24 - protein, 471 aa, Artificial Sequence
  FC6
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFKLYSKLTVDKSRWQQGNVFSCSVMHEALHNHYTQKSLSLSPGK

SEQ ID NO: 25 - protein, 471 aa, Artificial Sequence
  FC7
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSELTVDKSRWQQGNVFSCSVMHEALHNHYTQKSLSLSPGK

SEQ ID NO: 26 - protein, 471 aa, Artificial Sequence
  FC8
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 27 - protein, 471 aa, Artificial Sequence
  FC9
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPEVCTLPPSRDELTENQVSLWCLVKGFYPSDIAVEWESNGQPENNYKTTPPVLDSDGSFKLYSKLTVDKSRWQQGNVFSCSVMHEALHNHYTQKSLSLSPGK

SEQ ID NO: 28 - protein, 227 aa, Artificial Sequence
  FC10
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 29 - protein, 471 aa, Artificial Sequence
  FC11
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPRVCTLPPSRDELTKNQVSLWCLVKGFYPSDIAVEWESNGQPENNYKTTPPVLDSDGSFKLYSKLTVDKSRWQQGNVFSCSVMHEALHNHYTQKSLSLSPGK

SEQ ID NO: 30 - protein, 227 aa, Artificial Sequence
  FC12
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSRCELTENQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 31 - protein, 471 aa, Artificial Sequence
  FC13
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPEVCTLPPSRDELTENQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 32 - protein, 227 aa, Artificial Sequence
  FC14
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRCELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 33 - protein, 471 aa, Artificial Sequence
  FC15
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPRVCTLPPSRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 34 - protein, 227 aa, Artificial Sequence
  FC16
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSRCELTENQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 35 - protein, 471 aa, Artificial Sequence
  FC17
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCEVKGFYPSDIAVEWESNGQPENNYKTTPPVLDSDGSFFLYSALTVDKSRWQQGNVFSCSVMHEALHNHYTQKSLSLSPGK

SEQ ID NO: 36 - protein, 471 aa, Artificial Sequence
  FC18
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYDTTPPVLDSDGSFFLYSALTVDKSRWQQGNVFSCSVMHEALHNHYTQKSLSLSPGK

SEQ ID NO: 37 - protein, 227 aa, Artificial Sequence
  FC19
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLSSDGSFKLVSKLTVDKSRWQQGNVFSCSVMHEALHNHYTQKSLSLSPGK

SEQ ID NO: 38 - protein, 471 aa, Artificial Sequence
  FC20
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 39 - protein, 227 aa, Artificial Sequence
  FC21
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCGVKGFYPSDIAVEWESNGQPENNYKTTPPVLDSDGSFKLASKLTVDKSRWQQGNVFSCSVMHEALHNHYTQKSLSLSPGK

SEQ ID NO: 40 - protein, 227 aa, Artificial Sequence
  FC22
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDALTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 41 - protein, 471 aa, Artificial Sequence
  FC23
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVDTLPPDRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 42 - protein, 471 aa, Artificial Sequence
  FC24
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFKLYSKLTVDKSRWQQGNVFSCSVMHEALHNHYTQKSLSLSPGK

SEQ ID NO: 43 - protein, 227 aa, Artificial Sequence
  FC25
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSALTVDKSRWQQGNVFSCSVMHEALHNHYTQKSLSLSPGK

SEQ ID NO: 44 - protein, 471 aa, Artificial Sequence
  FC26
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLSSDGSFKLYSKLTVDKSRWQQGNVFSCSVMHEALHNHYTQKSLSLSPGK

SEQ ID NO: 45 - protein, 227 aa, Artificial Sequence
  FC27
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYDTTPPVLDSDGSFFLVSALTVDKSRWQQGNVFSCSVMHEALHNHYTQKSLSLSPGK

SEQ ID NO: 46 - protein, 227 aa, Artificial Sequence
  FC28
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCGVKGFYPSDIAVEWESNGQPENNYKTTPPVLDSDGSFFLASALTVDKSRWQQGNVFSCSVMHEALHNHYTQKSLSLSPGK

SEQ ID NO: 47 - protein, 471 aa, Artificial Sequence
  FC29
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVDTLPPSRDELTKNQVSLWCLVKGFYPSDIAVEWESNGQPENNYKTTPPVLDSDGSFKLYSKLTVDKSRWQQGNVFSCSVMHEALHNHYTQKSLSLSPGK

SEQ ID NO: 48 - protein, 227 aa, Artificial Sequence
  FC30
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDALTKNQVSLSCAVKGFYPSDIAVEWESNGQPENNYKTTPPVLDSDGSFFLVSALTVDKSRWQQGNVFSCSVMHEALHNHYTQKSLSLSPGK

SEQ ID NO: 49 - protein, 471 aa, Artificial Sequence
  FC31
EVQLLESGGGVVQPGRSLRLSCIASGFTFSSYPMTWVRQAPGKGLEWVASISYDGSYKYKADSMKGRLTISRDNSKNTLYLEMNSLTAEDTAVYYCARTAFFNAYDFWGQGTLVTVSSASTKGPSVGGGGSGGGGSEIVMTQSPATLSVSPGERATLSCRASQSVRSNLAWYQQKPGQAPRLLIYAASTRATGIPARFSGSGSGTEFTLTISSLQSEDFAVYYCQQYNEWFRTSGQGTKVEIKRDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVDTLPPDRDELTKNQVSLWCLVKGFYPSDIAVEWESNGQPENNYKTTPPVLDSDGSFKLYSKLTVDKSRWQQGNVFSCSVMHEALHNHYTQKSLSLSPGK

SEQ ID NO: 50 - protein, 330 aa, Homo sapiens [sequence omitted]

SEQ ID NO: 51 - protein, 227 aa, Artificial Sequence
  Fc 1-1
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 52 - protein, 227 aa, Artificial Sequence
  Fc 2-1
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

(19) United States
(12) Patent Application Publication
(10) Pub. No.: US 2023/0118053 A1
(43) Pub. Date: Apr. 20, 2023

(54) COMBINATION OF ANTI-HER2 ANTIBODY AND CDK INHIBITIOR FOR TUMOR TREATMENT

(71) Applicant: JIANGSU ALPHAMAB BIOPHARMACEUTICALS CO., LTD., Suzhou, Jiangsu (CN)

(72) Inventors: Ting XU, Suzhou, Jiangsu (CN); Junfang XU, Suzhou, Jiangsu (CN); Pilin WANG, Suzhou, Jiangsu (CN); Jing YANG, Suzhou, Jiangsu (CN); Jiazhu FANG, Suzhou, Jiangsu (CN); Lihong YUN, Suzhou, Jiangsu (CN); Yanrong DONG, Suzhou, Jiangsu (CN)

(21) Appl. No.: 17/914,916

(22) PCT Filed: Mar. 26, 2021
(86) PCT No.: PCT/CN2021/083309

§ 371 Date: Sep. 27, 2022

(30) Foreign Application Priority Data
Mar. 27, 2020 (CN) PCT/CN2020/081761
Mar. 27, 2020 (CN) PCT/CN2020/081773

(51) Int. Cl. A61K 39/395; A61K 31/566; A61P 35/00; A61P 35/04; A61K 31/519

(52) U.S. Cl. CPC A61K 39/39558; A61K 31/566; A61P 35/00; A61P 35/04; A61K 31/519

(57) ABSTRACT

The present application provides a medicinal product comprising: an the HER2 inhibitor or a CDK inhibitor, wherein said CDK inhibitor inhibits CDK4 and/or CDK6. The present application also provides an the HER2 inhibitor for the use of treating tumor in combination with a CDK inhibitor, as well as their use in the preparation of a medicament for treating tumor. The combination in present will significantly enhance tumor inhibiting.

### BACKGROUND OF THE INVENTION

HER2 belongs to a family of receptor tyrosine kinases leading to constitutive signaling through AKT and mTOR. Cyclin D1-CDK4/6 can participate in HER2 signaling by inducing the phosphorylation and inactivation of TSC2, an mTORC1 inhibitor. In preclinical models, CDK4/6 inhibitors display enhanced HER2 activity and increased sensitivity to HER2 kinase inhibitors or anti-HER2 antibodies.

And it is urgent and necessary to explore anti-tumor activity of dual HER2 blockade plus CDK4/6 inhibitor HER2-positive metastatic breast cancer (MBC) in clinical trial.

### SUMMARY OF THE INVENTION

The present application provides a medicinal product comprising: a HER2 inhibitor and a CDK inhibitor, wherein said CDK inhibitor inhibits CDK4 and/or CDK6. The present application also provides a HER2 inhibitor in combination with a CDK inhibitor of the present application for use in the preparation of a medicament for treating cancer in a subject in need thereof. The combination of the HER2 inhibitor in combination with the CDK inhibitor of the present application will significantly enhance tumor inhibiting.

In one aspect, the present application provides a medicinal product comprising: a HER2 inhibitor and a CDK inhibitor, wherein the HER2 inhibitor is capable of binding to a first HER2 antigen and a second HER2 antigen, and wherein the CDK inhibitor inhibits CDK4 and/or CDK6.

In some embodiments, the medicinal product is a composition.

In some embodiments, the HER2 is human HER2.

In some embodiments, the first HER2 antigen comprises an epitope within HER2 extracellular domain 2 (ECD2).

In some embodiments, the second HER2 antigen comprises an epitope within HER2 extracellular domain 2 (ECD2).

In some embodiments, the second HER2 antigen comprises an epitope within HER2 extracellular domain 4 (ECD4).

In some embodiments, the HER2 inhibitor is a bispecific antibody or an antigen binding portion thereof.

In some embodiments, the antigen binding portion thereof comprises Fab, Fab′, F(ab)₂, Fv fragment, F(ab′)₂, scFv, di-scFv and/or dAb.

In some embodiments, the HER2 inhibitor is a bispecific antibody or the antigen binding portion thereof, and the bispecific antibody or the antigen binding portion thereof has a first light chain and a second light chain, and wherein the first light chain and the second light chain comprises a same amino acid sequence.

In some embodiments, the HER2 inhibitor is a bispecific antibody or the antigen binding portion thereof, and the bispecific antibody or the antigen binding portion thereof has a first heavy chain and a second heavy chain, and wherein the first heavy chain and the second heavy chain are capable of correctly assembling with the light chains respectively under physiological conditions or during in vitro protein expression.

In some embodiments, the first light chain and the second light chain is capable of assembling with a heavy chain of Pertuzumab and a heavy chain of Trastuzumab, respectively.

In some embodiments, variable region of the first light chain and/or the second light chain comprises an amino acid sequence as set forth in any one of SEQ ID NO: 1-6.

In some embodiments, variable region of the first light chain and/or the second light chain comprises an amino acid sequence as set forth in SEQ ID NO: 1.

In some embodiments, the first light chain and the second light chain is selected from a light chain of Pertuzumab or a mutant thereof, a light chain of Trastuzumab or a mutant thereof, respectively.

In some embodiments, the first light chain comprises an amino acid sequence as set forth in any one of SEQ ID NO: 7-12, and/or, the second light chain comprises an amino acid sequence as set forth in any one of SEQ ID NO: 7-12.

In some embodiments, heavy chain variable regions are a heavy chain variable region of Pertuzumab and a heavy chain variable region of Trastuzumab, respectively.

In some embodiments, variable region of the first heavy chain comprises an amino acid sequence as set forth in SEQ ID NO: 13; and variable region of the second heavy chain comprises an amino acid sequence as set forth in SEQ ID NO: 14.

In some embodiments, the first heavy chain and the second heavy chain comprises a constant region, and the constant region is originated from human IgG constant region.

In some embodiments, Fc fragment sequences of the heavy chains comprise sequences as set forth in any one of SEQ ID NO: 19-49, 51-52.

In some embodiments, two heavy chains thereof comprise a sequence as set forth in any one of SEQ ID NO: 15-18.

In some embodiments, the CDK4 is human CDK4, and/or the CDK6 is human CDK6.

In some embodiments, the CDK inhibitor has a structure of formula I:

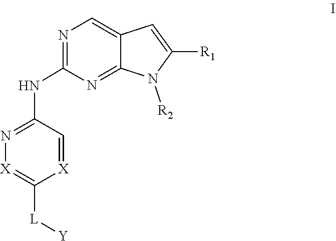

wherein X is CR⁹, or N; R¹ is C₁₋₈alkyl, CN, C(O)OR⁴ or CONR⁵R⁶, a 5-14 membered heteroaryl group, or a 3-14 membered cycloheteroalkyl group; R² is C₁₋₈alkyl, C₃_i₄cycloalkyl, or a 5-14 membered heteroaryl group, and wherein R² may be substituted with one or more C₁₋₈alkyl, or OH; L is a bond, C₁₋₈alkylene, C(O), or C(O)NR¹⁰, and wherein L may be substituted or unsubstituted; Y is H, R¹¹, NR¹²R¹³, OH, or Y is part of the following group

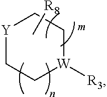

where Y is CR⁹ or N; where 0-3 R⁸ may be present, and R⁸ is C₁₋₈alkyl, oxo, halogen, or two or more R⁸ may form a bridged alkyl group; W is CR⁹, or N; R³ is H, C₁₋₈alkyl, C₁₋₈alkylR¹⁴, C₃-i₄cycloalkyl, C(O)C₁₋₈ alkyl, C₁₋₈haloalkyl, C₁₋₈alkylOH, C(O)NR¹⁴R¹⁵, Ci-gcyanoalkyl, C(O)R¹⁴, Co-₈alkylC(O)C₀₋₈alkylNR¹⁴R¹⁵, C₀₋₈alkylC(O)OR¹⁴, NR¹⁴R¹⁵, SO₂C₁₋₈alkyl, C₁₋₈alkylC₃-i₄cycloalkyl, C(O)C₁₋₈alkylC₃-i₄cycloalkyl, C₁₋₈alkoxy, or OH which may be substituted or unsubstituted when R³ is not H. R⁹ is H or halogen; R⁴, R⁵, R⁶, R⁷, R¹⁰, R¹¹, R¹², R¹³, R¹⁴, and R¹⁵ are each independently selected from H, C₁₋₈alkyl, C₃—H cycloalkyl, a 3-14 membered cycloheteroalkyl group, a Cβ-α aryl group, a 5-14 membered heteroaryl group, alkoxy, C(O)H, C(N)OH, C(N)OCH₃, C(O)C₁₋₃alkyl, C₁₋₈alkylNH₂, C₁₋₆ alkylOH, and wherein R⁴, R⁵, R⁶, R⁷, R¹⁰, R¹¹, R¹², and R¹³, R¹⁴, and R¹⁵ when not H may be substituted or unsubstituted; m and n are independently 0-2; and wherein L, R³, R⁴, R⁵, R⁶, R⁷, R¹⁰, R¹¹, R¹², and R¹³, R¹⁴, and R¹⁵ may be substituted with one or more of C₁₋₈alkyl, C₂₋₈alkenyl, C₂₋₈alkynyl, C3₋₁₄cycloalkyl, 5-14 membered heteroaryl group, C₆₋₁₄aryl group, a 3-14 membered cycloheteroalkyl group, OH, (O), CN, alkoxy, halogen, OrNH₂.

In some embodiments, R is H, C₁₋₈alkyl, C₃_i₄cycloalkyl, C(O)Ci-S alkyl, Ci-₈alkylOH, Ci-gcyanoalkyl, Co-₈alkylC(0)C₀-8alkylNR¹⁴R¹⁵, C₀₋₈alkylC(O)OR¹⁴, NR¹⁴R¹⁵, C₁₋₈alkylC_{3_}i₄cycloalkyl, C(O)C₁₋₈alkylC_{3_14}cycloalkyl, C₀₋₈alkoxy, C₁₋₈alkylR¹⁴, C₁₋₈haloalkyl, or C(O)R, which may be substituted with one or more of OH, CN, F, or NH₂, and wherein R and R are each independently selected from H, C₁₋₈alkyl, C₃_i₄cycloalkyl, alkoxy, C(O)C_{1_3}alkyl, C_{1_8}alkylNH₂, or C_{1_6}alkylOH.

In some embodiments, R is H, C₁₋₈alkyl, C₃_i₄cycloalkyl, C(O)Ci-S alkyl, Ci-₈alkylOH, Ci-gcyanoalkyl, Co-₈alkylC(O)C₀-8alkylNR¹⁴R¹⁵, C₀₋₈alkylC(O)OR¹⁴, NR¹⁴R¹⁵, C₁₋₈alkylC₃_i₄cycloalkyl, C(O)C₁₋₈alkylC_{3_14}cycloalkyl, C₀₋₈alkoxy, C₁₋₈alkylR¹⁴, C₁₋₈haloalkyl, or C(O)R, which may be substituted with one or more of OH, CN, F, or NH₂, and wherein R and R are each independently selected from H, C₁₋₈alkyl, C₃_i₄cycloalkyl, alkoxy, C(O)C_{1_3}alkyl, C_{1_8}alkylNH₂, or C_{1_6}alkylOH.

In some embodiments, Y is H, OH, or Y is part of the following group

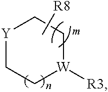

where Y is N and W is CR⁹, or N; where 0-2 R may be present, and R is C₁₋₈alkyl, oxo, or two or more R may form a bridged alkyl group.

In some embodiments, L is a bond, Ci-salkylene, or C(O)NH, or C(O).

In some embodiments, R² is C_{3_H}cycloalkyl.

In some embodiments, R is cyclopentane.

In some embodiments, R¹ is CN, C(O)OR⁴, CONR⁵R, or a 5-14 membered heteroaryl group.

In some embodiments, R is CONR R, a and R⁵ and R⁶ are C₁₋₈alkyl.

In some embodiments, X is CR.

In some embodiments, one X is N and the other X is CR.

In some embodiments, X is CR and Y is

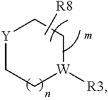

where m and n are 1, and Y and W are N.

In some embodiments, the CDK inhibitor has a structure of formula Ia:

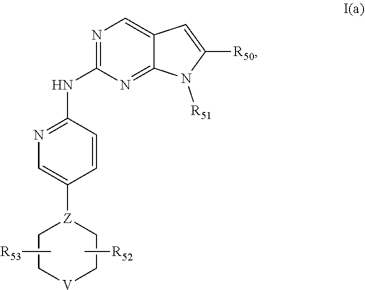

R⁵⁰ is CONR⁵⁴R⁵⁵, or CN; R⁵¹ is C_{3-H}cycloalkyl which may be unsubstituted or substituted by C₁₋₃alkyl, or OH; Z is CH or N; and V is NR⁵⁶ or CHR⁵⁷; R⁵⁴ and R⁵⁵ are independently H, C₁₋₃alkyl,
R⁵², R⁵³ R⁵⁶, and R⁵⁷ are independently H, C₁₋₈alkyl, C₃-i₄cycloalkyl, C₁₋₈haloalkyl, NR⁵⁸R⁵⁹, C(O)OR⁶⁰, C(O)C₁₋₈alkyl, Co-₈alkylC(O)C₀-8alkyl-NR⁶¹R⁶², C₁₋₈alkoxy, C₁₋₈alkyl0R⁶³, C(O)-5-Hcycloheteroalkyl group, C_{3-H}Cycloalkyl group, each of which when not H may be substituted by one or more of C₁₋₈alky, OH, or CN; R⁵⁸, R⁵⁹, R⁶⁰, R⁶¹, R⁶², and R⁶³ are H or C₁₋₈alkyl.

In some embodiments, the CDK inhibitor is selected from: 7-Cyclopentyl-2-[5-(3-methyl-piperazin-1-yl)-pyridin-2-ylamino]-7H-pyrrolo[2,3d]pyrimidine-6-carbonitrile; 7-Cyclopentyl-2-{5-[4-(2-fluoro-ethyl)-piperazin-1-yl]-pyridin-2-ylamino}-7H-pyrrolo[2,3-d]pyrimidine-6-carboxylic acid dimethylamide;7-Cyclopentyl-2-(4-dimethylamino-3,4,5,6-tetrahydro-2H-[1,3′]bipyridinyl-6′-ylamino)-7H-pyrrolo[2,3-d]pyrimidine-6-carboxylic acid dimethylamide;2-[5-(4-Carbamoylmethyl-piperazin-1-yl)-pyridin-2-ylamino]-7-cyclopentyl-7H-pyrrolo[2,3-d]pyrimidine-6-carboxylic acid dimethylamide;2-{5-[4-(2-Amino-acetyl)-piperazin-1-yl]-pyridin-2-ylamino}-7-cyclopentyl-7H-pyrrolo[2,3-d]pyrimidine-6-carboxylic acid dimethylamide;2-[5-(3-Amino-pyrrolidin-1-yl)-pyridin-2-ylamino]-7-cyclopentyl-7H-pyrrolo[2,3-d]pyrimidine-6-carboxylic acid dimethylamide;7-Cyclopentyl-2-{5-[4-(2-methoxy-ethyl)-piperazin-1-yl]-pyridin-2-ylamino}-7H-pyrrolo[2,3-d]pyrimidine-6-carboxylic acid dimethylamide;7-Cyclopentyl-2-[4-(2-hydroxyethyl)-3,4,5,6-tetrahydro-2H-[1,2′]bipyrazinyl-5′-ylamino]-7H-pyrrolo[2,3-d]pyrimidine-6-carboxylic acid dimethylamide; 7-Cyclopentyl-2-[5-((R)-3-methyl-piperazin-1-yl)-pyridin-2-ylamino]-7H-pyrrolo[2,3-d]pyrimidine-6-carboxylic acid dimethylamide;7-Cyclopentyl-2-[5-((S)-3-methylpiperazin-1-yl)-pyridin-2-ylamino]-7H-pyrrolo[2,3-d]pyrimidine-6-carboxylic acid dimethylamide;7-Cyclopentyl-2-[5-(3-methylpiperazin-1-yl)-pyridin-2-ylamino]-7H-pyrrolo[2,3-d]pyrimidine-6-carboxylic acid dimethylamide;7-Cyclopentyl-2-{5-[4-(3-hydroxypropyl)-piperazin-1-yl]-pyridin-2-ylamino}-7H-pyrrolo[2,3-d]pyrimidine-6-carboxylic acid dimethylamide;7-Cyclopentyl-2-{5-[4-(pyrrolidine-1-carbonyl)-piperazin-1-yl]-pyridin-2-ylamino}-7H-pyrrolo[2,3-d]pyrimidine-6-carboxylic acid dimethylamide;7-Cyclopentyl-2-{5-[4-(2-hydroxy-ethyl)-piperazin-1-yl]-pyridin-2-ylamino}-7H-pyrrolo[2,3-d]pyrimidine-6-carboxylic acid dimethylamide;7-Cyclopentyl-2-{5-[4-((S)-2,3-dihydroxypropyl)-piperazin-1-yl]-pyridin-2-ylamino}-7H-pyrrolo[2,3-d]pyrimidine-6-carboxylic acid dimethylamide; 7-Cyclopentyl-2-(5-{4-[2-(2-hydroxyethoxy)-ethyl]-piperazin-1-yl}-pyridin-2-ylamino)-7H-pyrrolo[2,3-d]pyrimidine-6-carboxylic acid dimethylamide; 7-Cyclopentyl-2-{5-[4-(2-hydroxy-1-methylethyl)-piperazin-1-yl]-pyridin-2-ylamino}-7H-pyrrolo[2,3-d]pyrimidine-6-carboxylic acid dimethylamide;7-Cyclopentyl-2-{6-[4-(2-hydroxyethyl)-piperazin-1-yl]-pyridazin-3-ylamino}-7H-pyrrolo [2,3-d]pyrimidine-6-carboxylic acid dimethylamide;7-Cyclopentyl-2-{5-[4-(2,3-dihydroxypropyl)-piperazin-1-yl]-pyridin-2-ylamino}-7H-pyrrolo[2,3-d]pyrimidine-6-carboxylic acid dimethylamide;7-Cyclopentyl-2-{5-[4-((R)-2,3-dihydroxypropyl)-piperazin-1-yl]-pyridin-2-ylamino}-7H-pyrrolo[2,3-d]pyrimidine-6-carboxylic acid dimethylamide;7-Cyclopentyl-2-(4-dimethylamino-3,4,5,6-tetrahydro-2H-[1,3′]bipyridinyl-6′-ylamino)-7H-pyrrolo[2,3-d]pyrimidine-6-carbonitrile;7-Cyclopentyl-2-(3,4,5,6-tetrahydro-2H-[1,2′]bipyrazinyl-5′-ylamino)-7H-pyrrolo[2,3-d]pyrimidine-6-carboxylic acid dimethylamide;7-Cyclopentyl-2-[5-(piperazine-1-carbonyl)-pyridin-2-ylamino]-7H-pyrrolo[2,3d]pyrimidine-6-carboxylic acid dimethylamide;7-Cyclopentyl-2-[5-(4-dimethylaminopiperidine-1-carbonyl)-pyridin-2-ylamino]-7H-pyrrolo[2,3-d]pyrimidine-6-carboxylic acid dimethylamide;7-Cyclopentyl-2-(1′,2^{>},3^{>},4^{>},5^{>},6′-hexahydro-[3,4^{>}]bipyridinyl-6-ylamino)-7H-pyrrolo[2,3d]pyrimidine-6-carboxylic acid dimethylamide;7-Cyclopentyl-2-[5-((S)-3-methylpiperazin-1-ylmethyl)-pyridin-2-ylamino]-7Hpyrrolo[2,3-d]pyrimidine-6-carboxylic acid dimethylamide;7-Cyclopentyl-2-{5-[4-((S)-2-hydroxypropyl)-piperazin-1-yl]-pyridin-2-ylamino}-7H-pyrrolo[2,3-d]pyrimidine-6-carboxylic acid dimethylamide;7-Cyclopentyl-2-{5-[4-((R)-2-hydroxypropyl)-piperazin-1-yl]-pyridin-2-ylamino}-7H-pyrrolo [2,3-d]pyrimidine-6-carboxylic acid dimethylamide;7-Cyclopentyl-2-(5-piperazin-1-yl-pyridin-2-ylamino)-7H-pyrrolo[2,3-d]pyrimidine-6-carboxylic acid methylamide;7-Cyclopentyl-2-[5-(4-isopropyl-piperazin-1-yl)-pyridin-2-ylamino]-7H-pyrrolo[2,3d]pyrimidine-6-carboxylic acid dimethylamide;7-Cyclopentyl-2-[5-(4-isopropyl-piperazine-1-carbonyl)-pyridin-2-ylamino]-7H-pyrrolo[2,3-d]pyrimidine-6-carboxylic acid dimethylamide; 7-Cyclopentyl-2-{5-[4-(4-methyl-pentyl)-piperazin-1-yl]-pyridin-2-ylamino}-7H-pyrrolo[2,3-d]pyrimidine-6-carboxylic acid dimethylamide;7-Cyclopentyl-2-[6-(4-isopropyl-piperazin-1-yl)-pyridazin-3-ylamino]-7H-pyrrolo[2,3-d]pyrimidine-6-carboxylic acid dimethylamide;7-Cyclopentyl-2-{5-[4-(2-hydroxy-2methylpropyl)-piperazin-1-yl]-pyridin-2-ylamino}-7H-pyrrolo[2,3-d]pyrimidine-6-carboxylic acid dimethylamide;7-Cyclopentyl-2-[5-(3,3-dimethyl-piperazin-1-yl)-pyridin-2-ylamino]-7H-pyrrolo[2,3-d]pyrimidine-6-carboxylic acid dimethylamide;7-Cyclopentyl-2-[5-(3,8-diaza-bicyclo[3.2.1]oct-3-ylmethyl)-pyridin-2-ylamino]-7H-pyrrolo[2,3-d]pyrimidine-6-carboxylic acid dimethylamide;7-Cyclopentyl-2-(5-piperazin-1-yl-pyridin-2-ylamino)-7H-pyrrolo[2,3-d]pyrimidine-6-carboxylic acid dimethylamide;7-Cyclopentyl-2-[5-(4-ethyl-piperazin-1-yl)-pyridin-2-ylamino]-7H-pyrrolo[2,3-d]pyrimidine-6-carboxylic acid dimethylamide;7-Cyclopentyl-2-[5-(4-cyclopentyl-piperazin-1-yl)-pyridin-2-ylamino]-7H-pyrrolo[2,3-d]pyrimidine-6-carboxylic acid dimethylamide;7-Cyclopentyl-2-(1′-isopropyl-1′,2′,3′,4′,5′,6′-hexahydro-[3,4′]bipyridinyl-6-ylamino)-7H-pyrrolo[2,3-d]pyrimidine-6-carboxylic acid dimethylamide;7-Cyclopentyl-2-{5-[(R)-4-(2-hydroxyethyl)-3-methyl-piperazin-1-yl]-pyridin-2-ylamino}-7H-pyrrolo[2,3-d]pyrimidine-6-carboxylic acid dimethylamide;7-Cyclopentyl-2-{5-[(S)-4-(2-hydroxy ethyl)-3-methyl-piperazin-1-yl]-pyridin-2-ylamino}-7H-pyrrolo[2,3-d]pyrimidine-6-carboxylic acid dimethylamide;7-Cyclopentyl-2-{5-[4-(2-hydroxyethyl)-piperazin-1-ylmethyl]-pyridin-2-ylamino}-7H-pyrrolo[2,3-d]pyrimidine-6-carboxylic acid dimethylamide;7-Cyclopentyl-2-{5-[4-(2-dimethylaminoacetyl)-piperazin-1-yl]-pyridin-2-ylamino}-7H-pyrrolo[2,3-d]pyrimidine-6-carboxylic acid dimethylamide;7-Cyclopentyl-2-{5-[4-(2-ethyl-butyl)piperazin-1-yl]-pyridin-2-ylamino}-7H-pyrrolo[2,3-d]pyrimidine-6-carboxylic acid dimethylamide;2-{5-[4-(2-Cyclohexyl-acetyl)piperazin-1-yl]-pyridin-2-ylamino}-7-cyclopentyl-7H-pyrrolo[2,3-d]pyrimidine-6-carboxylic acid dimethylamide; 7-Cyclopentyl-2-{5-[4-(3-cyclopentyl-propionyl)-piperazin-1-yl]-pyridin-2-ylamino}7H-pyrrolo[2,3-d]pyrimidine-6-carboxylic acid dimethylamide;7-Cyclopentyl-2-[5-(4-isobutylpiperazin-1-yl)-pyridin-2-ylamino]-7H-pyrrolo[2,3d]pyrimidine-6-carboxylic acid dimethylamide;{4-[6-(7-Cyclopentyl-6-dimethylcarbamoyl-7H-pyrrolo[2,3-d]pyrimidin-2-ylamino)pyridin-3-yl]-piperazin-1-yl}-acetic acid methyl ester;7-Cyclopentyl-2-{5-[4-(2-isopropoxyethyl)-piperazin-1-yl]-pyridin-2-ylamino}-7Hpyrrolo[2,3-d]pyrimidine-6-carboxylic acid dimethylamide;{4-[6-(7-Cyclopentyl-6-dimethylcarbamoyl-7H-pyrrolo[2,3-d]pyrimidin-2-ylamino)pyridin-3-yl]-piperazin-1-yl}-acetic acid ethyl ester;4-(6-{7-Cyclopentyl-6-[(2-hydroxy-ethyl)methyl-carbamoyl]-7H-pyrrolo[2,3-d]pyrimidin-2-ylamino}-pyridin-3-yl)piperazine-1-carboxylic acid tert-butyl ester;7-Cyclopentyl-2-{5-[4-(2-methyl-butyl)piperazin-1-yl]-pyridin-2-ylamino}-7H-pyrrolo[2,3-d]pyrimidine-6-carboxylic acid dimethylamide;7-Cyclopentyl-2-[1′-(2-hydroxy-ethyl)-1′,2′,3′,4′,5′,6′-hexahydro-[3,4′]bipyridinyl-6-ylamino]-7H-pyrrolo[2,3-d]pyrimidine-6-carboxylic acid dimethylamide;{4-[6-(7-Cyclopentyl-6-dimethylcarbamoyl-7H-pyrrolo[2,3-d]pyrimidin-2-ylamino)-pyridin-3-yl]piperazin-1-yl}-acetic acid; and 2-{4-[6-(7-Cyclopentyl-6-dimethylcarbamoyl-7H-pyrrolo[2,3-d]pyrimidin-2-ylamino)-pyridin-3-yl]-piperazin-1-yl}-propionic acid; or pharmaceutically acceptable salts thereof.

In some embodiments, the CDK inhibitor has a structure of formula II:

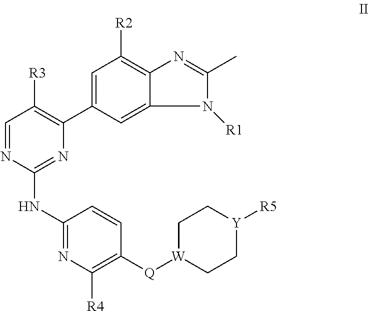

R1 is C₃-C₅ alkyl, C₃-C₅ cycloalkyl or cyclopropyl-methyl; R2 and R3 are H or fluorine, wherein at least one of R2 or R3 is fluorine; R4 is H or CH₃; R5 is C₁-C₆ alkyl or —NR6R7 wherein R6 and R7 are C₁-C₃ alkyl; Q is CH₂, O, S or a direct bond; and W and Y are C or N, wherein at least one of W or Y is N and wherein when Q is O or S, W is C; or a pharmaceutically acceptable salt thereof.

In some embodiments, R1 is isopropyl, cyclopropyl, cyclopentyl or cyclopropyl-methyl.

In some embodiments, R1 is isopropyl.

In some embodiments, R2 and R3 are each fluorine.

In some embodiments, R4 is H.

In some embodiments, R5 is C₁-C₃ alkyl.

In some embodiments, Q is CH₂ or a direct bond.

In some embodiments, Q is CH₂.

In some embodiments, Y is N.

In some embodiments, W is N.

In some embodiments, the CDK inhibitor is selected from the group consisting of:

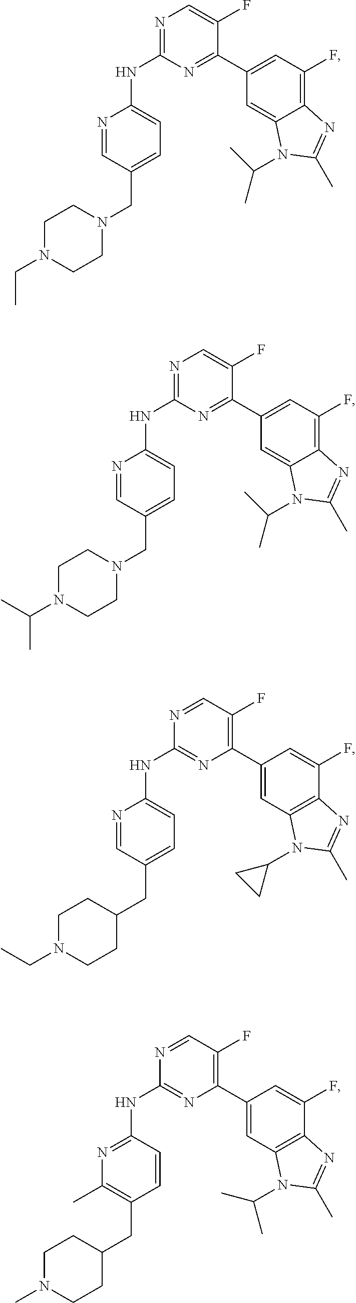 [SMILES: CCN1CCN(Cc2ccc(Nc3ncc(F)c(-c4cc(F)c5nc(C)n(C(C)C)c5c4)n3)nc2)CC1 ; Cc1nc2c(F)cc(-c3nc(Nc4ccc(CN5CCN(C(C)C)CC5)cn4)ncc3F)cc2n1C(C)C ; CCN1CCC(Cc2ccc(Nc3ncc(F)c(-c4cc(F)c5nc(C)n(C6CC6)c5c4)n3)nc2)CC1 ; Cc1nc(Nc2ncc(F)c(-c3cc(F)c4nc(C)n(C(C)C)c4c3)n2)ccc1CC1CCN(C)CC1]

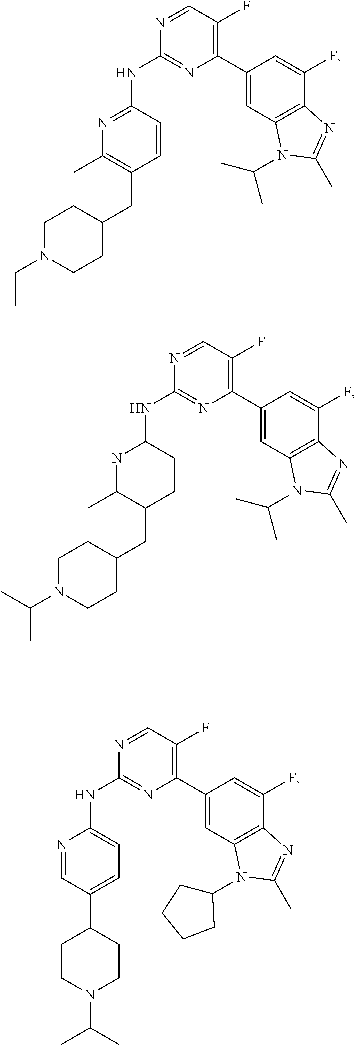 [SMILES: CCN1CCC(Cc2ccc(Nc3ncc(F)c(-c4cc(F)c5nc(C)n(C(C)C)c5c4)n3)nc2C)CC1 ; Cc1nc2c(F)cc(-c3nc(NC4CCC(CC5CCN(C(C)C)CC5)C(C)N4)ncc3F)cc2n1C(C)C ; Cc1nc2c(F)cc(-c3nc(Nc4ccc(C5CCN(C(C)C)CC5)cn4)ncc3F)cc2n1C1CCCC1]

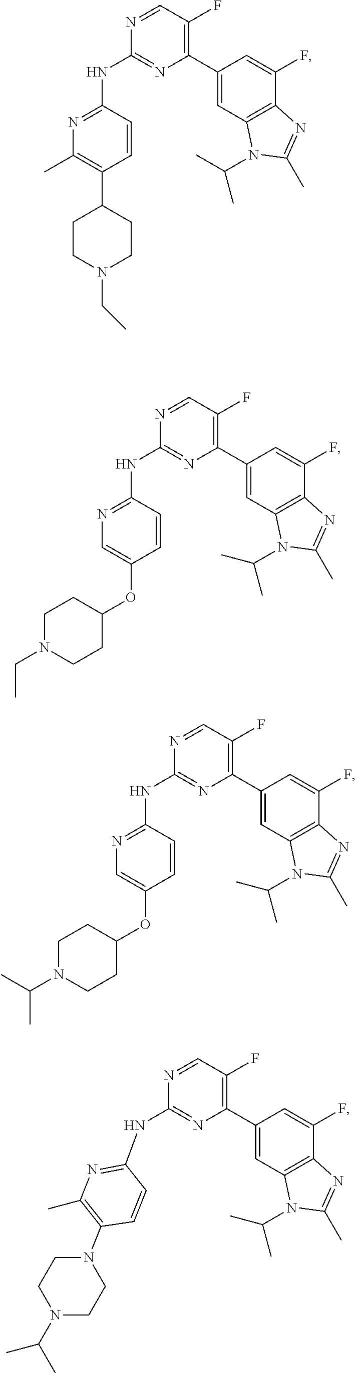 [SMILES: CCN1CCC(c2ccc(Nc3ncc(F)c(-c4cc(F)c5nc(C)n(C(C)C)c5c4)n3)nc2C)CC1 ; CCN1CCC(Oc2ccc(Nc3ncc(F)c(-c4cc(F)c5nc(C)n(C(C)C)c5c4)n3)nc2)CC1 ; Cc1nc2c(F)cc(-c3nc(Nc4ccc(OC5CCN(C(C)C)CC5)cn4)ncc3F)cc2n1C(C)C ; Cc1nc(Nc2ncc(F)c(-c3cc(F)c4nc(C)n(C(C)C)c4c3)n2)ccc1N1CCN(C(C)C)CC1]

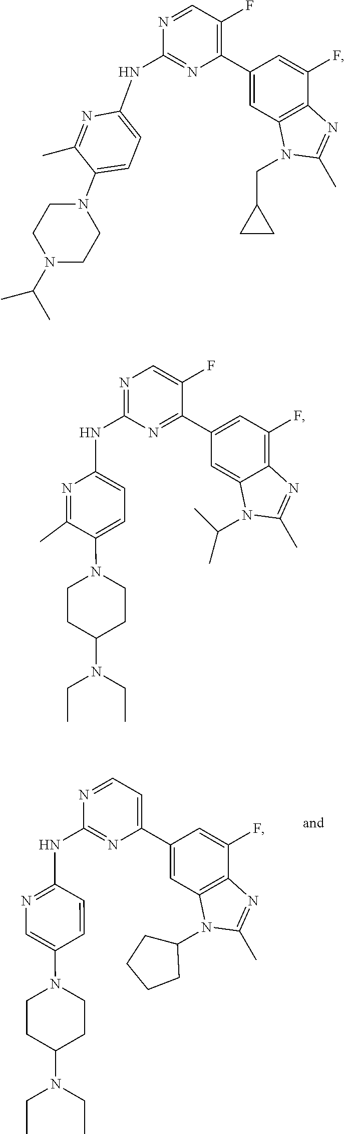 [SMILES: Cc1nc(Nc2ncc(F)c(-c3cc(F)c4nc(C)n(CC5CC5)c4c3)n2)ccc1N1CCN(C(C)C)CC1 ; CCN(CC)C1CCN(c2ccc(Nc3ncc(F)c(-c4cc(F)c5nc(C)n(C(C)C)c5c4)n3)nc2C)CC1 ; CCN(CC)C1CCN(c2ccc(Nc3nccc(-c4cc(F)c5nc(C)n(C6CCCC6)c5c4)n3)nc2)CC1]

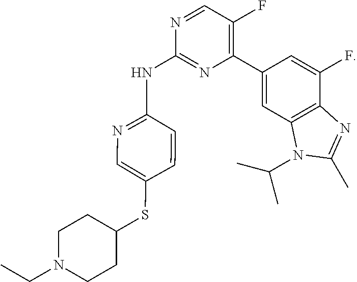 [SMILES: CCN1CCC(Sc2ccc(Nc3ncc(F)c(-c4cc(F)c5nc(C)n(C(C)C)c5c4)n3)nc2)CC1]

In some embodiments, the CDK inhibitor is

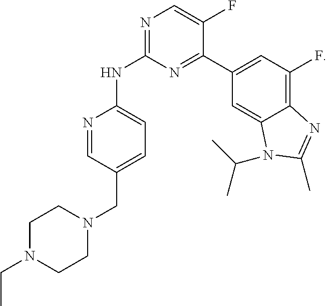 [SMILES: CCN1CCN(Cc2ccc(Nc3ncc(F)c(-c4cc(F)c5nc(C)n(C(C)C)c5c4)n3)nc2)CC1]

In some embodiments, the CDK inhibitor has a structure of formula III,

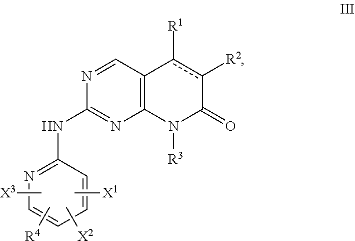

wherein the HER2 inhibitor comprises a first antigen binding domain and a second antigen binding domain, the first antigen binding domain is capable of specifically binding to a first HER2 antigen, the second antigen binding domain is capable of specifically binding to a second HER2 antigen, and the first HER2 antigen and the second HER2 antigen are not identical; X¹, X², and X³ are independently hydrogen, halogen, C₁-C₆ alkyl, C₁-C₆ halo alkyl, C₁-C₈ alkoxy, C₁-C₈ alkoxyalkyl, CN, NO₂, OR⁵, NR⁵R⁶, CO₂R⁵, COR⁵, S(O)ₙR⁵, CONR⁵R⁶, NR⁵COR⁶, NR⁵SO₂R⁶, SO₂NR⁵R⁶, and P(O)(OR⁵)(OR⁶); with the proviso that at least one of X¹, X², and X³ must be hydrogen; n is selected from 0, 1 and 2; R¹ is, in each instance, independently, hydrogen, halogen, C₁-C₆ alkyl, C₁-C₆ halo alkyl, C₁-C₆ hydroxyalkyl, or C₃-C₇ cycloalkyl; R² and R⁴ are each independently selected from hydrogen, halogen, C₁-C₈ alkyl, C₃-C₇ cycloalkyl, C₁-C₈ alkoxy, C₁-C₈ alkoxyalkyl, C₁-C₈ halo alkyl, C₁-C₈ hydroxyalkyl, C₂-C₈ alkenyl, C₂-C₈ alkynyl, nitrile, nitro, OR⁵, SR⁵, NR⁵R⁶, N(O)R⁵R⁶, P(O)(OR⁵)(OR⁶), (CR⁵R⁶)ₘNR⁷R⁸, COR⁵, (CR⁴R⁵)ₘC(O)R⁷, CO₂R⁵, CONR⁵R⁶, C(O)NR⁵SO₂R⁶, NR⁵SO₂R⁶, C(O)NR⁵OR⁶, S(O)ₙR⁵, SO₂NR⁵R⁶, P(O)(OR⁵)(OR⁶), (CR⁵R⁶)ₘP(O)(OR⁷)(OR⁸), (CR⁵R⁶)ₘ-aryl, (CR⁵R⁶)ₘ-heteroaryl, T(CH₂)ₘQR⁵, —C(O)T(CH₂)ₘQR⁵, NR⁵C(O)T(CH₂)ₘQR⁵, and —CR⁵═CR⁶C(O)R⁷; or R¹ and R² may form a carbocyclic group containing 3-7 ring members, preferably 5-6 ring members, up to four of which can optionally be replaced with a heteroatom independently selected from oxygen, sulfur, and nitrogen, and wherein the carbocyclic group is unsubstituted or substituted with one, two, or three groups independently selected from halogen, hydroxy, hydroxyalkyl, nitrile, lower C₁-C₈ alkyl, lower C₁-C₈ alkyl, alkoxycarbonyl, alkylcarbonyl, alkylcarbonylamino, aminoalkyl, trifluoromethyl, N-hydroxyacetamide, trifluoromethylalkyl, amino, and mono or dialkylamino, (CH₂)ₘC(O)NR⁵R⁶, and O(CH₂)ₘC(O)OR⁵, provided, however, that there is at least one carbon atom in the carbocyclic ring and that if there are two or more ring oxygen atoms, the ring oxygen atoms are not adjacent to one another; T is O, S, NR⁷, N(O)R⁷, NR⁷R⁸W, or CR⁷R⁸; Q is O, S, NR⁷, N(O)R⁷, NR⁷R⁸W, CO₂, O(CH₂)ₘ-heteroaryl, O(CH₂)ₘS(O)ₙR⁸, (CH₂)-heteroaryl, or a carbocyclic group containing from 3-7 ring members, up to four of which ring members are optionally heteroatoms independently selected from oxygen, sulfur, and nitrogen, provided, however, that there is at least one carbon atom in the carbocyclic ring and that if there are two or more ring oxygen atoms, the ring oxygen atoms are not adjacent to one another, wherein the carbocyclic group is unsubstituted or substituted with one, two, or three groups independently selected from alkoxycarbonyl, alkylcarbonyl, alkylcarbonylamino, aminoalkyl, trifluoromethyl, N-hydroxyacetamide, trifluoromethylalkyl, amino, and mono or dialkylamino; W is an anion selected from the group consisting of chloride, bromide, trifluoroacetate, and triethylammonium; m is selected from 0, 1, 2, 3, 4, 5 and 6; R⁴ and one of X¹, X² and X³ may form an aromatic ring containing up to three heteroatoms independently selected from oxygen, sulfur, and nitrogen, and optionally substituted by up to 4 groups independently selected from alogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, mono- or dialkylamino, N-hydroxyacetamido, aryl, heteroaryl, carboxyalkyl, nitrile, NR⁷SO₂R⁸, C(O)NR⁷R⁸, NR⁷C(O)R⁸, C(O)OR⁷, C(O)NR⁷SO₂R⁸, (CH₂)ₘS(O)ₙR⁷, (CH₂)ₘ-heteroaryl, O(CH₂)ₘ-heteroaryl, (CH₂), C(O)NR⁷R⁸, O(CH₂)ₘC(O)OR⁷, (CH₂)ₘSO₂NR⁷R⁸, and C(O)R⁷; R³ is hydrogen, aryl, C₁-C₈ alkyl, C₁-C₈ alkyl, C₃-C₇ cycloalkyl, or C₃-C₇-heterocyclyl; R⁵ and R⁶ are each independently hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl; or R⁵ and R⁶, when attached to the same nitrogen atom, taken together with the nitrogen to which they are attached, form a heterocyclic ring containing from 3-8 ring members, up to four of which members can optionally be replaced with heteroatoms independently selected from oxygen, sulfur, S(O), S(O)₂, and nitrogen, provided, however, that there is at least one carbon atom in the heterocyclic ring and that if there are two or more ring oxygen atoms, the ring oxygen atoms are not adjacent to one another, wherein the heterocyclic group is unsubstituted or substituted with one, two or three groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, nitrile, mono- or dialkylamino, N-hydroxyacetamido, aryl, heteroaryl, carboxyalkyl, NR⁷SO₂R⁸, C(O)NR⁷R⁸, NR⁷C(O)R⁸, C(O)OR⁷, C(O)NR⁷SO₂R⁸, (CH₂)ₘS(O)ₙR⁷, (CH₂)ₘ-heteroaryl, O(CH₂)ₘ-heteroaryl, (CH₂)ₘC(O)NR⁷R⁸, O(CH₂)ₘC(O)OR⁷, and (CH₂)SO₂NR⁷R⁸; R⁷ and R⁸ are, each independently, hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl; or R⁷ and R⁸, when attached to the same nitrogen atom, taken together with the nitrogen to which they are attached, may form a heterocyclic ring containing from 3-8 ring members, up to four of which members are optionally heteroatoms independently selected from oxygen, sulfur, S(O), S(O)₂, and nitrogen, provided, however, that there is at least one carbon atom in the heterocyclic ring and that if there are two or more ring oxygen atoms, the ring oxygen atoms are not adjacent to one another, wherein the heterocyclic group is unsubstituted or substituted with one, two or three groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, nitrile, mono- or dialkylamino, N-hydroxyacetamido, aryl, heteroaryl, carboxyalkyl; and the pharmaceutically acceptable salts, esters, amides, and prodrugs thereof.

In some embodiments, the compound has a structure of formula IV:

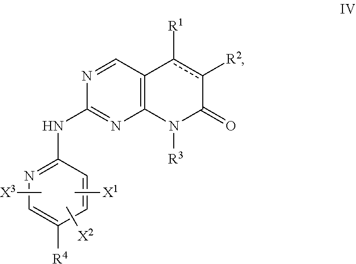

wherein X¹, X², and X³ are independently hydrogen, halogen, C₁-C₆ alkyl, C₁-C₆ halo alkyl, C₁-C₈ alkoxy, C₁-C₈ alkoxyalkyl, CN, NO₂, OR⁵, NR⁵R⁶, CO₂R⁵, COR⁵, S(O)ₙR⁵, CONR⁵R⁶, NR⁵COR⁶, NR⁵SO₂R⁶, SO₂NR⁵R⁶, and P(O)(OR⁵)(OR⁶); with the proviso that at least one of X¹, X², and X³ must be hydrogen; n is selected from 0, 1 and 2; R¹ is, in each instance, independently, hydrogen, halogen, C₁-C₆ alkyl, C₁-C₆ halo alkyl, C₁-C₆ hydroxyalkyl, or C₃-C₇ cycloalkyl; R² and R⁴ are each independently selected from hydrogen, halogen, C₁-C₈ alkyl, C₃-C₇ cycloalkyl, C₁-C₈ alkoxy, C₁-C₈ alkoxyalkyl, C₁-C₈ halo alkyl, C₁-C₈ hydroxyalkyl, C₂-C₈ alkenyl, C₂-C₈ alkynyl, nitrile, nitro, OR⁵, SR⁵, NR⁵R⁶, N(O)R⁵R⁶, P(O)(OR⁵)(OR⁶), (CR⁵R⁶)ₘNR⁷R⁸, COR⁵, (CR⁴R⁵)ₘC(O)R⁷, CO₂R⁵, CONR⁵R⁶, C(O)NR⁵SO₂R⁶, NR⁵SO₂R⁶, C(O)NR⁵OR⁶, S(O)ₙR⁵, SO₂NR⁵R⁶, P(O)(OR⁵)(OR⁶), (CR⁵R⁶)ₘP(O)(OR⁷)(OR⁸), (CR⁵R⁶)ₘ-aryl, (CR⁵R⁶)ₘ-heteroaryl, T(CH₂)ₘQR⁵, —C(O)T(CH₂)ₘQR⁵, NR⁵C(O)T(CH₂)ₘQR⁵, and —CR⁵═CR⁶C(O)R⁷; or R¹ and R² may form a carbocyclic group containing 3-7 ring members, preferably 5-6 ring members, up to four of which can optionally be replaced with a heteroatom independently selected from oxygen, sulfur, and nitrogen, and wherein the carbocyclic group is unsubstituted or substituted with one, two, or three groups independently selected from halogen, hydroxy, hydroxyalkyl, nitrile, lower C₁-C₈ alkyl, lower C₁-C₈ alkyl, alkoxycarbonyl, alkylcarbonyl, alkylcarbonylamino, aminoalkyl, trifluoromethyl, N-hydroxyacetamide, trifluoromethylalkyl, amino, and mono or dialkylamino, (CH₂)ₘC(O)NR⁵R⁶, and O(CH₂)ₘC(O)OR⁵, provided, however, that there is at least one carbon atom in the carbocyclic ring and that if there are two or more ring oxygen atoms, the ring oxygen atoms are not adjacent to one another; T is O, S, NR⁷, N(O)R⁷, NR⁷R⁸W, or CR⁷R⁸; Q is O, S, NR⁷, N(O)R⁷, NR⁷R⁸W, CO₂, O(CH₂)ₘ-heteroaryl, O(CH₂)ₘS(O)ₙR⁸, (CH₂)-heteroaryl, or a carbocyclic group containing from 3-7 ring members, up to four of which ring members are optionally heteroatoms independently selected from oxygen, sulfur, and nitrogen, provided, however, that there is at least one carbon atom in the carbocyclic ring and that if there are two or more ring oxygen atoms, the ring oxygen atoms are not adjacent to one another, wherein the carbocyclic group is unsubstituted or substituted with one, two, or three groups independently selected from alkoxycarbonyl, alkylcarbonyl, alkylcarbonylamino, aminoalkyl, trifluoromethyl, N-hydroxyacetamide, trifluoromethylalkyl, amino, and mono or dialkylamino; W is an anion selected from the group consisting of chloride, bromide, trifluoroacetate, and triethylammonium; m is selected from 0, 1, 2, 3, 4, 5 and 6; R⁴ and one of X¹, X² and X³ may form an aromatic ring containing up to three heteroatoms independently selected from oxygen, sulfur, and nitrogen, and optionally substituted by up to 4 groups independently selected from alogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, mono- or dialkylamino, N-hydroxyacetamido, aryl, heteroaryl, carboxyalkyl, nitrile, NR⁷SO₂R⁸, C(O)NR⁷R⁸, NR⁷C(O)R⁸, C(O)OR⁷, C(O)NR⁷SO₂R⁸, (CH₂)ₘS(O)ₙR⁷, (CH₂)ₘ-heteroaryl, O(CH₂)ₘ-heteroaryl, (CH₂), C(O)NR⁷R⁸, O(CH₂)ₘC(O)OR⁷, (CH₂)ₘSO₂NR⁷R⁸, and C(O)R⁷; R³ is hydrogen, aryl, C₁-C₈ alkyl, C₁-C₈ alkyl, C₃-C₇ cycloalkyl, or C₃-C₇-heterocyclyl; R⁵ and R⁶ are each independently hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl; or R⁵ and R⁶, when attached to the same nitrogen atom, taken together with the nitrogen to which they are attached, form a heterocyclic ring containing from 3-8 ring members, up to four of which members can optionally be replaced with heteroatoms independently selected from oxygen, sulfur, S(O), S(O)₂, and nitrogen, provided, however, that there is at least one carbon atom in the heterocyclic ring and that if there are two or more ring oxygen atoms, the ring oxygen atoms are not adjacent to one another, wherein the heterocyclic group is unsubstituted or substituted with one, two or three groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, nitrile, mono- or dialkylamino, N-hydroxyacetamido, aryl, heteroaryl, carboxyalkyl, NR⁷SO₂R⁸, C(O)NR⁷R⁸, NR⁷C(O)R⁸, C(O)OR⁷, C(O)NR⁷SO₂R⁸, (CH₂)ₘS(O)ₙR⁷, (CH₂)ₘ-heteroaryl, O(CH₂)ₘ-heteroaryl, (CH₂)ₘC(O)NR⁷R⁸, O(CH₂)ₘC(O)OR⁷, and (CH₂)SO₂NR⁷R⁸; R⁷ and R⁸ are, each independently, hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl; or R⁷ and R⁸, when attached to the same nitrogen atom, taken together with the nitrogen to which they are attached, may form a heterocyclic ring containing from 3-8 ring members, up to four of which members are optionally heteroatoms independently selected from oxygen, sulfur, S(O), S(O)₂, and nitrogen, provided, however, that there is at least one carbon atom in the heterocyclic ring and that if there are two or more ring oxygen atoms, the ring oxygen atoms are not adjacent to one another, wherein the heterocyclic group is unsubstituted or substituted with one, two or three groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, nitrile, mono- or dialkylamino, N-hydroxyacetamido, aryl, heteroaryl, carboxyalkyl; and the pharmaceutically acceptable salts, esters, amides, and prodrugs thereof.

In some embodiments, R³ is a cyclopentyl group.

In some embodiments, R¹ is alkyl.

In some embodiments, R¹ is methyl.

In some embodiments, R² is (CO)CH₃.

In some embodiments, the compound is selected from a group consisting of: 8-Cyclopentyl-2-(pyridin-2-ylamino)-8H-pyrido[2,3-d]pyrimidin-7-one, 6-Bromo-8-cyclopentyl-2-(5-piperazin-1-yl-pyridin-2-ylamino)-8H-pyrido [2,3-d]pyrimidin-7-one hydrochloride, 8-Cyclopentyl-6-ethyl-2-(5-piperazin-1-yl-pyridin-2-ylamino)-8H-pyrido[2,3-d]pyrimidin-7-one hydrochloride, 8-Cyclopentyl-7-oxo-2-(5-piperazin-1-yl-pyridin-2-ylamino)-7,8-dihydro-pyrido[2,3-d]pyrimidine-6-carboxylic acid ethyl ester hydrochloride, 6-Amino-8-cyclopentyl-2-(5-piperazin-1-yl-pyridin-2-ylamino)-8H-pyrido[2,3-d]pyrimidin-7-one hydrochloride, 6-Bromo-8-cyclopentyl-2-[5-((R)-1-methy-1-pyrrolidin-2-yl)-pyridin-2-ylamino]-8H-pyrido[2,3-d]pyrimidin-7-one hydrochloride, 6-Bromo-8-cyclohexyl-2-(pyridin-2-yl-amino)-8H-pyrido[2,3-d]pyrimidin-7-one, 6-Acetyl-8-cyclopentyl-2-[5-(3,5-dimethyl-piperazin-1-yl)-pyridin-2-ylamino]-5-methyl-8H-pyrido[2,3-d]pyrimidin-7-one, 6-Acetyl-8-cyclopentyl-2-[5-(3,3-dimethyl-piperazin-1-yl)-pyridin-2-ylamino]-5-methyl-8H-pyrido[2,3-d]pyrimidin-7-one, 6-Acetyl-8-cyclopentyl-5-methyl-2-[5-(4-methyl-piperazin-1-yl)-pyridin-2-ylamino]-8H-pyrido[2,3-d]pyrimidin-7-one, 6-Acetyl-2-[5-(3-amino-pyrrolidin-1-yl)-pyridin-2-ylamino]-8-cyclopentyl-5-methyl-8H-pyrido[2,3-d]pyrimidin-7-one, 6-Bromo-8-cyclopentyl-5-methyl-2-(5-morpholin-4-yl-pyridin-2-ylamino)-8H-pyrido[2,3-d]pyrimidin-7-one, 2-{5-[Bis-(2-methoxy-ethyl)-amino]-pyridin-2-ylamino}-6-bromo-8-cyclopentyl-5-methyl-8H-pyrido[2,3-d]pyrimidin-7-one, 6-Acetyl-8-cyclopentyl-5-methyl-2-(5-morpholin-4-yl-pyridin-2-ylamino)-8H-pyrido[2,3-d]pyrimidin-7-one, 6-Acetyl-2-{5-[bis-(2-methoxy-ethyl)-amino]-pyridin-2-ylamino}-8-cyclopentyl-5-methyl-8H-pyrido[2,3-d]pyrimidin-7-one, 4-[6-(8-Cyclopentyl-6-iodo-5-methyl-7-oxo-7,8-dihydro-pyrido[2,3-d]pyrimidin-2-ylamino)-pyridin-3-yl]-piperazine-1-carboxylic acid tert-butyl ester, 8-Cyclopentyl-6-iodo-5-methyl-2-(5-piperazin-1-yl-pyridin-2-ylamino)-8H-pyrido[2,3-d]pyrimidin-7-one, 4-{6-[8-Cyclopentyl-6-(2-ethoxy-ethoxy)-7-oxo-7,8-dihydro-pyrido[2,3-d]pyrimidin-2-ylamino]-pyridin-3-yl}-piperazine-1-carboxylic acid tert-butyl ester, 8-Cyclopentyl-6-(2-ethoxy-ethoxy)-2-(5-piperazin-1-yl-pyridin-2-ylamino)-8H-pyrido[2,3-d]pyrimidin-7-one, 2-{5-[Bis-(2-methoxy-ethyl)-amino]-pyridin-2-ylamino}-6-bromo-8-cyclopentyl-5-methyl-8H-pyrido[2,3-d]pyrimidin-7-one, 6-Acetyl-2-{5-[bis-(2-methoxy-ethyl)-amino]-pyridin-2-ylamino}-8-cyclopentyl-5-methyl-8H-pyrido[2,3-d]pyrimidin-7-one, 4-[6-(8 isopropyl-7-oxo-7,8-dihydro-pyrido[2,3]pyrimidin-2-ylamino)-pyridin-3-yl]-piperazine-1-carboxylic acid tert-butyl ester, 8-isopropyl-2-(5-piperazin-1-yl-pyridin-2-ylamino)-8H-pyrido[2,3-d]pyrimidin-7-one, 4-[6-(8-cyclopentyl-7-oxox-7,8-dihydro-pyrido[2,3-d]pyrimidin-2-ylamino)-pyridin-3-yl]-piperazine-1-carboxylic acid tert-butyl ester, 8-cyclopentyl-2-(5-piperazin-1-yl-pyridin-2-ylamino)-8H-pyrido[2,3-d]pyrimidin-7-one, 4-[6-(8-cyclohexyl-7-oxo-7,8-dihydro-pyrido[2,3-d]pyrimidin-2-ylamino)-pyridin-3-yl]-piperazine-1-carboxylic acid tert-butyl ester, 8-cyclohexyl-2-(5-piperazin-1-yl-pyridin-2-ylamino)-8H-pyrido[2,3-d]pyrimidin-7-one, 4-[6-(8-cyclopropyl-7-oxo-7,8-dihydro-pyrido[2,3-d]pyrimidin-2-ylamino)-pyridin-3-yl]-piperazine-1-carboxylic acid tert-butyl ester, 8-cyclopropyl-2-(5-piperazin-1-yl-pyridin-2-ylamino)-8H-pyrido[2,3-d]pyrimidin-7-one, 6-Bromo-8-cyclopentyl-2-(pyridin-2,6-yldiamino)-8H-pyrido[2,3-d]pyrimidin-7-one, 6-Bromo-8-cyclopentyl-5-methyl-2-(pyridin-2-ylamino)-8H-pyrido[2,3-d]pyrimidin-7-one, 6-Bromo-8-cyclopentyl-5-methyl-2-[5-(4-methyl-piperazin-1-yl)-pyridin-2-ylamino]-8H-pyrido[2,3-d]pyrimidin-7-one, 8-Cyclopentyl-6-(1-ethoxy-vinyl)-5-methyl-2-[5-(4-methyl-piperazin-1-yl)-pyridin-2-ylamino]-8H-pyrido[2,3-d]pyrimidin-7-one, (1-{6-[8-Cyclopentyl-6-(1-ethoxy-vinyl)-5-methyl-7-oxo-7,8-dihydro-pyrido[2,3-d]pyrimidin-2-ylamino]-pyridin-3-yl}-pyrrolidin-3-yl)-carbamic acid tert-butyl ester, 6-Acetyl-8-cyclopentyl-2-(4-hydroxy-3,4,5,6-tetrahydro-2H-[1,3′]bipyridinyl-6′-ylamino)-5-methyl-8H-pyrido[2,3-d]pyrimidin-7-one, 4-[6-(6-Bromo-8-cyclopentyl-5-methyl-7-oxo-7,8-dihydro-pyrido[2,3-d]pyrimidin-2-ylamino)-pyridin-3-yl]-azepane-1-carboxylic acid tert-butyl ester, 6-Bromo-8-cyclopentyl-2-(5-[1,4]diazepan-1-yl-pyridin-2-ylamino)-5-methyl-8H-pyrido[2,3-d]pyrimidin-7-one, 4-{6-[8-Cyclopentyl-6-(1-ethoxy-vinyl)-5-methyl-7-oxo-7,8-dihydro-pyrido[2,3-d]pyrimidin-2-ylamino]-pyridin-3-yl}-[1,4]diazepane-1-carboxylic acid tert-butyl ester, 6-Acetyl-8-cyclopentyl-2-(5-[1,4]diazepan-1-yl-pyridin-2-ylamino)-5-methyl-8H-pyrido[2,3-d]pyrimidin-7-one, 6-Acetyl-8-cyclopentyl-5-methyl-2-(pyridin-2-ylamino)-8H-pyrido[2,3-d]pyrimidin-7-one, 4-[6-(8-Cyclopentyl-5-methyl-7-oxo-7,8-dihydro-pyrido[2,3-d]pyrimidin-2-ylamino)-pyridin-3-yl]-piperazine-1-carboxylic acid tert-butyl ester, 8-Cyclopentyl-5-methyl-2-(5-piperazin-4-yl-pyridin-2-ylamino)-8H-pyrido[2,3-d]pyrimidin-7-one, 4-[6-(6-Bromo-8-cyclopentyl-5-methyl-7-oxo-7,8-dihydro-pyrido[2,3-d]pyrimidin-2-ylamino)-pyridin-3-yl]-2,2-dimethyl-piperazine-1-carboxylic acid tert-butyl ester, 6-Bromo-8-cyclopentyl-2-[5-(3,3-dimethyl-piperazin-1-yl)-pyridin-2-ylamino]-5-methyl-8H-pyrido[2,3d]pyrimidin-7-one, 4-{6-[8-Cyclopentyl-6-(1-ethoxy-vinyl)-5-methyl-7-oxo-7,8-dihydro-pyrido[2,3-d]pyrimidin-2-ylamino]-pyridin-3-yl}-2,2-dimethyl-piperazine-1-carboxylic acid tert-butyl ester, 4-[6-(6-Bromo-8-cyclopentyl-5-methyl-7-oxo-7,8-dihydro-pyrido[2,3-d]pyrimidin-2-ylamino)-pyridin-3-yl]-2,6-dimethyl-piperazine-1-carboxylic acid tert-butyl ester, 6-Bromo-8-cyclopentyl-2-[5-(3,5-dimethyl-piperazin-1-yl)-pyridin-2-ylamino]-5-methyl-8H-pyrido[2,3-d]pyrimidin-7-one, 4-{6-[8-Cyclopentyl-6-(1-ethoxy-vinyl)-5-methyl-7-oxo-7,8-dihydro-pyrido[2,3-d]pyrimidin-2-ylamino]-pyridin-3-yl}-2,6-dimethyl-piperazine-1-carboxylic acid tert-butyl ester, 8-Cyclopentyl-6-(1-ethoxy-vinyl)-5-methyl-2-(5-morpholin-4-yl-pyridin-2-ylamino)-8H-pyrido[2,3-d]pyrimidin-7-one, 6-Bromo-8-cyclopentyl-5-methyl-2-(3,4,5,6-tetrahydro-2H-[1,3′]bipyridinyl-6′-ylamino)-8H-pyrido[2,3-d]pyrimidin-7-one, 8-Cyclopentyl-6-(1-ethoxy-vinyl)-5-methyl-2-(3,4,5,6-tetrahydro-2H-[1,3′]bipyridinyl-6′-ylamino)-8H-pyrido[2,3-d]pyrimidin-7-one, 6-Acetyl-8-cyclopentyl-5-methyl-2-(3,4,5,6-tetrahydro-2H-[1,3′]bipyridinyl-6′-ylamino)-8H-pyrido[2,3-d]pyrimidin-7-one, 4-{6-[8-Cyclopentyl-6-(2-ethoxy-ethyl)-7-oxo-7,8-dihydro-pyrido[2,3-d]pyrimidin-2-ylamino]-pyridin-3-yl}-piperazine-1-carboxylic acid tert-butyl ester, 8-Cyclopentyl-6-(2-ethoxy-ethyl)-2-(5-piperazin-1-yl-pyridin-2-ylamino)-8H-pyrido[2,3-d]pyrimidin-7-one, 4-{6-[8-Cyclopentyl-6-(2-methoxy-ethoxymethyl)-7-oxo-7,8-dihydro-pyrido[2,3-d]pyrimidin-2-ylamino]-pyridin-3-yl}-piperazine-1-carboxylic acid tert-butyl ester, 8-Cyclopentyl-6-(2-methoxy-ethoxymethyl)-2-(5-piperazin-1-yl-pyridin-2-ylamino)-8H-pyrido[2,3-d]pyrimidin-7-one, 4-[6-(8-Cyclopentyl-6-ethoxymethyl-7-oxo-7,8-dihydro-pyrido[2,3-d]pyrimidin-2-ylamino)-pyridin-3-yl]-piperazine-1-carboxylic acid tert-butyl ester, 8-Cyclopentyl-6-ethoxymethyl-2-(5-piperazin-1-yl-pyridin-2-ylamino)-8H-pyrido[2,3-d]pyrimidin-7-one, 4-[6-(8-Cyclopentyl-6-methoxymethyl-7-oxo-7,8-dihydro-pyrido[2,3-d]pyrimidin-2-ylamino)-pyridin-3-yl]-piperazine-1-carboxylic acid tert-butyl ester, 8-Cyclopentyl-6-methoxymethyl-2-(5-piperazin-1-yl-pyridin-2-ylamino)-8H-pyrido[2,3-d]pyrimidin-7-one, 6-Bromo-8-cyclopentyl-2-[5-(2,6-dimethyl-morpholin-4-yl)-pyridin-2-ylamino]-5-methyl-8H-pyrido[2,3-d]pyrimidin-7-one, 8-Cyclopentyl-6-ethoxymethyl-2-(3,4,5,6-tetrahydro-2H-[1,3′]bipyridinyl-6′-ylamino)-8H-pyrido[2,3-d]pyrimidin-7-one, 8-Cyclopentyl-6-ethoxymethyl-2-(5-morpholin-4-yl-pyridin-2-ylamino)-8H-pyrido[2,3-d]pyrimidin-7-one, [8-Cyclopentyl-7-oxo-2-(3,4,5,6-tetrahydro-2H-[1,3′]bipyridinyl-6′-ylamino)-7,8-dihydro-pyrido[2,3-d]pyrimidin-6-ylmethyl]-carbamic acid benzyl ester, 8-Cyclopentyl-2-[5-(2,6-dimethyl-morpholin-4-yl)-pyridin-2-ylamino]-6-(1-ethoxy-vinyl)-5-methyl-8H-pyrido[2,3-d]pyrimidin-7-one, 6-Acetyl-8-cyclopentyl-2-[5-(2,6-dimethyl-morpholin-4-yl)-pyridin-2-ylamino]-5-methyl-8H-pyrido[2,3-d]pyrimidin-7-one, 8-Cyclopentyl-5-methyl-2-(5-piperazin-1-yl-pyridin-2-ylamino)-6-propionyl-8H-pyrido[2,3-d]pyrimidin-7-one, 6-Bromo-8-cyclopentyl-2-methyl-8H-pyrido[2,3-d]pyrimidin-7-one, 6-Bromo-8-cyclopentyl-5-methyl-2-(5-piperazin-1-yl-pyridin-2-ylamino)-8H-pyrido[2,3-d]pyrimidin-7-one, 8-Cyclopentyl-6-fluoro-2-(5-piperazin-1-yl-pyridin-2-ylamino)-8H-pyrido[2,3-d]pyrimidin-7-one hydrochloride, 8-Cyclopentyl-6-methyl-2-(5-piperazin-1-yl-pyridin-2-ylamino)-8H-pyrido[2,3-d]pyrimidin-7-one hydrochloride, 8-Cyclopentyl-6-isobutoxy-2-(5-piperazin-1-yl-pyridin-2-ylamino)-8H-pyrido[2,3-d]pyrimidin-7-one hydrochloride, 6-Benzyl-8-cyclopentyl-2-(5-piperazin-1-yl-pyridin-2-ylamino)8H-pyrido[2,3-d]pyrimidin-7-one hydrochloride, 8-Cyclopentyl-6-hydroxymethyl-2-(5-piperazin-1-yl-pyridin-2-ylamino)-8H-pyrido[2,3-d]pyrimidin-7-one hydrochloride, 2-[5-(4-tert-Butoxycarbonyl-piperazin-1-yl)-pyridin-2-ylamino]-8-cyclopentyl-5-methyl-7-oxo-7,8-dihydro-pyrido[2,3-d]pyrimidine-6-carboxylic acid ethyl ester, 6-Acetyl-8-cyclopentyl-2-(5-piperazin-1-yl-pyridin-2-ylamino)-8H-pyrido[2,3-d]pyrimidin-7-one, 6-Acetyl-8-cyclopentyl-5-methyl-2-(5-piperazin-1-yl-pyridin-2-ylamino)-8H-pyrido[2,3-d]pyrimidin-7-one, 6-Bromo-8-cyclopentyl-5-methyl-2-(pyridin-2-ylamino)-8H-pyrido[2,3-d]pyrimidin-7-one, 6-Bromo-8-cyclopentyl-2-(pyridin-2-ylamino)-8H-pyrido[2,3-d]pyrimidin-7-one, 6-Bromo-8-cyclopentyl-2-[5-(3,5-dimethyl-piperazin-1-yl)-pyridin-2-ylamino]-8H-pyrido[2,3-d]pyrimidin-7-one, 6-Bromo-8-cyclopentyl-2-[5-(3,3-dimethyl-piperazin-1-yl)-pyridin-2-ylamino]-8H-pyrido[2,3-d]pyrimidin-7-one, 6-Bromo-8-cyclopentyl-2-[5-(4-methyl-piperazin-1-yl)-pyridin-2-ylamino]-8H-pyrido[2,3-d]pyrimidin-7-one, 2-[5-(3-Amino-pyrrolidin-1-yl)-pyridin-2-ylamino]-6-bromo-8-cyclopentyl-8H-pyrido[2,3-d]pyrimidin-7-one, 6-Bromo-8-cyclopentyl-2-[5-(3-ethylamino-pyrrolidin-1-yl)-pyridin-2-ylamino]-8H-pyrido[2,3-d]pyrimidin-7-one, 6-Bromo-8-cyclopentyl-2-(5-pyrrolidin-1-yl-pyridin-2-ylamino)-8H-pyrido[2,3-d]pyrimidin-7-one, 2-{5-[3-(1-Amino-1-methyl-ethyl)-pyrrolidin-1-yl]-pyridin-2-ylamino}-6-bromo-8-cyclopentyl-8H-pyrido[2,3-d]pyrimidin-7-one, 1-[6-(6-Bromo-8-cyclopentyl-7-oxo-7,8-dihydro-pyrido[2,3-d]pyrimidin-2-ylamino)-pyridin-3-yl]-pyrrolidine-2-carboxylic acid, 6-Bromo-8-cyclopentyl-2-[5-(4-diethylamino-butylamino)-pyridin-2-ylamino]-8H-pyrido[2,3-d]pyrimidin-7-one, 6-Acetyl-8-cyclopentyl-2-[5-(3-ethylamino-pyrimidin-1-yl)-pyridin-2-ylamino]-5-methyl-8H-pyrido[2,3-d]pyrimidin-7-one, 6-Acetyl-8-cyclopentyl-5-methyl-2-(5-pyrrolidin-1-yl-pyridin-2-ylamino)-8H-pyrido[2,3-d]pyrimidin-7-one, 6-Acetyl-2-{5-[3-(1-amino-1-methyl-ethyl)-pyrrolidin-1-yl]-pyridin-2-ylamino}-8-cyclopentyl-5-methyl-8H-pyrido[2,3-d]pyrimidin-7-one, 1-[6-(6-Acetyl-8-cyclopentyl-5-methyl-7-oxo-7,8-dihydro-pyrido[2,3-d]pyrimidin-2-ylamino)-pyridin-3-yl]-pyrrolidine-2-carboxylic acid, 6-Acetyl-8-cyclopentyl-2-[5-(4-diethylamino-butylamino)-pyridin-2-ylamino]-5-methyl-8H-pyrido[2,3-d]pyrimidin-7-one, 8-Cyclopentyl-2-[5-(3,5-dimethyl-piperazin-1-yl)-pyridin-2-ylamino]-6-ethyl-8H-pyrido[2,3-d]pyrimidin-7-one, 8-Cyclopentyl-2-[5-(3,3-dimethyl-piperazin-1-yl)-pyridin-2-ylamino]-6-ethyl-8H-pyrido[2,3-d]pyrimidin-7-one, 8-Cyclopentyl-6-ethyl-2-[5-(4-methyl-piperazin-1-yl)-pyridin-2-ylamino]-8H-pyrido[2,3-d]pyrimidin-7-one, 2-[5-(3-Amino-pyrrolidin-1-yl)-pyridin-2-ylamino]-8-cyclopentyl-6-ethyl-8H-pyrido[2,3-d]pyrimidin-7-one, 8-Cyclopentyl-6-ethyl-2-[5-(3-ethylamino-pyrrolidin-1-yl)-pyridin-2-ylamino]-8H-pyrido[2,3-d]pyrimidin-7-one, 8-Cyclopentyl-6-ethyl-2-(5-pyrrolidin-1-yl-pyridin-2-ylamino)-8H-pyrido[2,3-d]pyrimidin-7-one, 2-{5-[3-(1-Amino-1-methyl-ethyl)-pyrrolidin-1-yl]-pyridin-2-ylamino}-8-cyclopentyl-6-ethyl-8H-pyrido[2,3-d]pyrimidin-7-one, 1-[6-(8-Cyclopentyl-6-ethyl-7-oxo-7,8-dihydro-pyrido[2,3-d]pyrimidin-2-ylamino)-pyridin-3-yl]-pyrrolidine-2-carboxylic acid, 8-Cyclopentyl-2-[5-(4-diethylamino-butylamino)-pyridin-2-ylamino]-6-ethyl-8H-pyrido[2,3-d]pyrimidin-7-one, 6-Benzyl-8-cyclopentyl-2-[5-(3,5-dimethyl-piperazin-1-yl)-pyridin-2-ylamino]-8H-pyrido[2,3-d]pyrimidin-7-one, 6-Benzyl-8-cyclopentyl-2-[5-(3,3-dimethyl-piperazin-1-yl)-pyridin-2-ylamino]-8H-pyrido[2,3-d]pyrimidin-7-one, 6-Benzyl-8-cyclopentyl-2-[5-(4-methyl-piperazin-1-yl)-pyridin-2-ylamino]-8H-pyrido[2,3d]pyrimidin-7-one, 2-[5-(3-Amino-pyrrolidin-1-yl)-pyridin-2-ylamino]-6-benzyl-8-cyclopentyl-8H-pyrido[2,3-d]pyrimidin-7-one, 6-Benzyl-8-cyclopentyl-2-[5-(3-ethylamino-pyrrolidin-1-yl)-pyridin-2-ylamino]-8H-pyrido[2,3-d]pyrimidin-7-one, 6-Benzyl-8-cyclopentyl-2-(5-pyrrolidin-1-yl-pyridin-2-ylamino)-8H-pyrido[2,3-d]pyrimidin-7-one, 2-{5-[3-(1-Amino-1-methyl-ethyl)-pyrrolidin-1-yl]-pyridin-2-ylamino}-6-benzyl-8-cyclopentyl-8H-pyrido[2,3-d]pyrimidin-7-one, 1-[6-(6-Benzyl-8-cyclopentyl-7-oxo-7,8-dihydro-pyrido[2,3-d]pyrimidin-2-ylamino)-pyridin-3-yl]-pyrrolidine-2-carboxylic acid, 6-Benzyl-8-cyclopentyl-2-[5-(4-diethylamino-butylamino)-pyridin-2-ylamino]-8H-pyrido[2,3-d]pyrimidin-7-one, 8-Cyclopentyl-2-[5-(3,5-dimethyl-piperazin-1-yl)-pyridin-2-ylamino]-6-hydroxymethyl-8H-pyrido[2,3-d]pyrimidin-7-one, 8-Cyclopentyl-2-[5-(3,3-dimethyl-piperazin-1-yl)-pyridin-2-ylamino]-6-hydroxymethyl-8H-pyrido[2,3-d]pyrimidin-7-one, 8-Cyclopentyl-6-hydroxymethyl-2-[5-(4-methyl-piperazin-1-yl)-pyridin-2-ylamino]-8H-pyrido[2,3-d]pyrimidin-7-one, 2-[5-(3-Amino-pyrrolidin-1-yl)-pyridin-2-ylamino]-8-cyclopentyl-6-hydroxymethyl-8H-pyrido[2,3-d]pyrimidin-7-one, 8-Cyclopentyl-2-[5-(3-ethylamino-pyrrolidin-1-yl)-pyridin-2-ylamino]-6-hydroxymethyl-8H-pyrido[2,3-d]pyrimidin-7-one, 8-Cyclopentyl-6-hydroxymethyl-2-(5-pyrrolidin-1-yl-pyridin-2-ylamino)-8H-pyrido[2,3-d]pyrimidin-7-one, 2-{5-[3-(1-Amino-1-methyl-ethyl)-pyrrolidin-1-yl]-pyridin-2-ylamino}-8-cyclopentyl-6-hydroxymethyl-8H-pyrido[2,3-d]pyrimidin-7-one, 1-[6-(8-Cyclopentyl-6-hydroxymethyl-7-oxo-7,8-dihydro-pyrido[2,3-d]pyrimidin-2-ylamino)-pyridin-3-yl]-pyrrolidine-2-carboxylic acid, 8-Cyclopentyl-2-[5-(4-diethylamino-butylamino)-pyridin-2-ylamino]-6-hydroxymethyl-8H-pyrido[2,3-d]pyrimidin-7-one, 6-Amino-8-cyclopentyl-2-[5-(3,5-dimethyl-piperazin-1-yl)-pyridin-2-ylamino]-8H-pyrido[2,3-d]pyrimidin-7-one, 6-Amino-8-cyclopentyl-2-[5-(3,3-dimethyl-piperazin-1-yl)-pyridin-2-ylamino]-8H-pyrido[2,3-d]pyrimidin-7-one, 6-Amino-8-cyclopentyl-2-[5-(4-methyl-piperazin-1-yl)-pyridin-2-ylamino]-8H-pyrido[2,3d]pyrimidin-7-one, 6-Amino-2-[5-(3-amino-pyrrolidin-1-yl)-pyridin-2-ylamino]-8-cyclopentyl-8H-pyrido[2,3-d]pyrimidin-7-one, 6-Amino-8-cyclopentyl-2-[5-(3-ethylamino-pyrrolidin-1-yl)-pyridin-2-ylamino]-8H-pyrido[2,3-d]pyrimidin-7-one, 6-Amino-8-cyclopentyl-2-(5-pyrrolidin-1-yl-pyridin-2-ylamino)-81H-pyrido[2,3-d]pyrimidin-7-one, 6-Amino-2-{5-[3-(1-amino-1-methyl-ethyl)-pyrrolidin-1-yl]-pyridin-2-ylamino}-8-cyclopentyl-8H-pyrido[2,3-d]pyrimidin-7-one, 1-[6-(6-Amino-8-cyclopentyl-7-oxo-7,8-dihydro-pyrido[2,3-d]pyrimidin-2-ylamino)-pyridin-3-yl]-pyrrolidine-2-carboxylic acid, 6-Amino-8-cyclopentyl-2-[5-(4-diethylamino-butylamino)-pyridin-2-ylamino]-8H-pyrido[2,3-d]pyrimidin-7-one, 6-Bromo-8-cyclopentyl-2-(3,4,5,6-tetrahydro-2H-[1,3′]bipyridinyl-6′-ylamino)-8H-pyrido[2,3-d]pyrimidin-7-one, 6-Bromo-8-cyclopentyl-2-(5-morpholin-4-yl-pyridin-2-ylamino)-8H-pyrido[2,3-d]pyrimidin-7-one, 6-Bromo-8-cyclopentyl-2-(5-diethylamino-pyridin-2-ylamino)-8H-pyrido[2,3-d]pyrimidin-7-one, 2-{5-[Bis-(2-hydroxy-ethyl)-amino]-pyridin-2-ylamino}-6-bromo-8-cyclopentyl-8H-pyrido[2,3-d]pyrimidin-7-one, 2-{5-[Bis-(2-methoxy-ethyl)-amino]-pyridin-2-ylamino}-6-bromo-8-cyclopentyl-8H-pyrido[2,3-d]pyrimidin-7-one, 2-[5-(2-Amino-ethylamino)-pyridin-2-ylamino]-6-bromo-8-cyclopentyl-8H-pyrido[2,3-d]pyrimidin-7-one, 6-Bromo-8-cyclopentyl-2-(5-dimethylamino-pyridin-2-ylamino)-8H-pyrido[2,3-d]pyrimidin-7-one, N-[6-(6-Bromo-8-cyclopentyl-7-oxo-7,8-dihydro-pyrido[2,3-d]pyrimidin-2-ylamino)-pyridin-3-yl]-N-methyl-acetamide, 6-Bromo-8-cyclopentyl-2-[5-(2-methoxy-ethoxy)-pyridin-2-ylamino]-8H-pyrido[2,3-d]pyrimidin-7-one, 6-Bromo-8-cyclopentyl-2-[5-(2-methoxy-ethoxymethyl)-pyridin-2-ylamino]-8H-pyrido[2,3-d]pyrimidin-7-one, 6-Bromo-8-cyclopentyl-2-[5-(2-diethylamino-ethoxy)-pyridin-2-ylamino]-8H-pyrido[2,3-d]pyrimidin-7-one, 6-Bromo-8-cyclopentyl-2-(5-pyrrolidin-1-yl-pyridin-2-ylamino)-8H-pyrido[2,3-d]pyrimidin-7-one, 6-Bromo-8-cyclopentyl-2-(6-methyl-5-piperazin-1-yl-pyridin-2-ylamino)-8H-pyrido[2,3-d]pyrimidin-7-one, 6-Bromo-8-cyclopentyl-5-methyl-2-(3,4,5,6-tetrahydro-2H-[1,3′]bipyridinyl-6′-ylamino)-8H-pyrido[2,3-d]pyrimidin-7-one, 6-Bromo-8-cyclopentyl-2-(5-diethylamino-pyridin-2-ylamino)-5-methyl-8H-pyrido[2,3-d]pyrimidin-7-one, 2-{5-[Bis-(2-hydroxy-ethyl)-amino]-pyridin-2-ylamino}-6-bromo-8-cyclopentyl-5-methyl-8H-pyrido[2,3-d]pyrimidin-7-one, 2-[5-(2-Amino-ethylamino)-pyridin-2-ylamino]-6-bromo-8-cyclopentyl-5-methyl-8H-pyrido[2,3-d]pyrimidin-7-one, 6-Bromo-8-cyclopentyl-2-(5-dimethylamino-pyridin-2-ylamino)-5-methyl-8H-pyrido[2,3-d]pyrimidin-7-one, N-[6-(6-Bromo-8-cyclopentyl-5-methyl-7-oxo-7,8-dihydro-pyrido[2,3-d]pyrimidin-2-ylamino)-pyridin-3-yl]-N-methyl-acetamide, 6-Bromo-8-cyclopentyl-2-[5-(2-methoxy-ethoxy)-pyridin-2-ylamino]-5-methyl-8H-pyrido[2,3-d]pyrimidin-7-one, 6-Bromo-8-cyclopentyl-2-[5-(2-methoxy-ethoxymethyl)-pyridin-2-ylamino]-5-methyl-8H-pyrido[2,3-d]pyrimidin-7-one, 6-Bromo-8-cyclopentyl-2-[5-(2-diethylamino-ethoxy)-pyridin-2-ylamino]-5-methyl-8H-pyrido[2,3-d]pyrimidin-7-one, 6-Bromo-8-cyclopentyl-5-methyl-2-(5-pyrrolidin-1-yl-pyridin-2-ylamino)-8H-pyrido[2,3-d]pyrimidin-7-one, 6-Bromo-8-cyclopentyl-5-methyl-2-(6-methyl-5-piperazin-1-yl-pyridin-2-ylamino)-8H-pyrido[2,3-d]pyrimidin-7-one, 6-Acetyl-8-cyclopentyl-5-methyl-2-(3,4,5,6-tetrahydro-2H-[1,3′]bipyridinyl-6′-ylamino)-8H-pyrido[2,3-d]pyrimidin-7-one, 6-Acetyl-8-cyclopentyl-2-(5-diethylamino-pyridin-2-ylamino)-5-methyl-8H-pyrido[2,3-d]pyrimidin-7-one, 6-Acetyl-2-{5-[bis-(2-hydroxy-ethyl)-amino]-pyridin-2-ylamino}-8-cyclopentyl-5-methyl-8H-pyrido[2,3-d]pyrimidin-7-one, 6-Acetyl-2-[5-(2-amino-ethylamino)-pyridin-2-ylamino]-8-cyclopentyl-5-methyl-8H-pyrido[2,3-d]pyrimidin-7-one, 6-Acetyl-8-cyclopentyl-2-(5-dimethylamino-pyridin-2-ylamino)-5-methyl-8H-pyrido[2,3-d]pyrimidin-7-one, N-[6-(6-Acetyl-8-cyclopentyl-5-methyl-7-oxo-7,8-dihydro-pyrido[2,3-d]pyrimidin-2-ylamino)-pyridin-3-yl]-N-methyl-acetamide, 6-Acetyl-8-cyclopentyl-2-[5-(2-methoxy-ethoxy)-pyridin-2-ylamino]-5-methyl-8H-pyrido[2,3-d]pyrimidin-7-one, 6-Acetyl-8-cyclopentyl-2-[5-(2-methoxy-ethoxymethyl)-pyridin-2-ylamino]-5-methyl-8H-pyrido[2,3-d]pyrimidin-7-one, 6-Acetyl-8-cyclopentyl-2-[5-(2-diethylamino-ethoxy)-pyridin-2-ylamino]-5-methyl-8H-pyrido[2,3-d]pyrimidin-7-one, 6-Acetyl-8-cyclopentyl-5-methyl-2-(5-pyrrolidin-1-yl-pyridin-2-ylamino)-8H-pyrido[2,3-d]pyrimidin-7-one, 6-Acetyl-8-cyclopentyl-5-methyl-2-(6-methyl-5-piperazin-1-yl-pyridin-2-ylamino)-8H-pyrido[2,3-d]pyrimidin-7-one, 6-Acetyl-8-cyclopentyl-2-(3,4,5,6-tetrahydro-2H-[1,3′]bipyridinyl-6′-ylamino)-8H-pyrido[2,3-d]pyrimidin-7-one, 6-Acetyl-8-cyclopentyl-2-(5-morpholin-4-yl-pyridin-2-ylamino)-8H-pyrido[2,3-d]pyrimidin-7-one, 6-Acetyl-8-cyclopentyl-2-(5-diethylamino-pyridin-2-ylamino)-8H-pyrido[2,3-d]pyrimidin-7-one, 6-Acetyl-2-{5-[bis-(2-hydroxy-ethyl)-amino]-pyridin-2-ylamino}-8-cyclopentyl-8H-pyrido[2,3-d]pyrimidin-7-one, 6-Acetyl-2-{5-[bis-(2-methoxy-ethyl)-amino]-pyridin-2-ylamino}-8-cyclopentyl-8H-pyrido[2,3-d]pyrimidin-7-one, 6-Acetyl-2-[5-(2-amino-ethylamino)-pyridin-2-ylamino]-8-cyclopentyl-8H-pyrido[2,3-d]pyrimidin-7-one, 6-Acetyl-8-cyclopentyl-2-(5-dimethylamino-pyridin-2-ylamino)-8H-pyrido[2,3-d]pyrimidin-7-one, N-[6-(6-Acetyl-8-cyclopentyl-7-oxo-7,8-dihydro-pyrido[2,3-d]pyrimidin-2-ylamino)-pyridin-3-yl]-N-methyl-acetamide, 6-Acetyl-8-cyclopentyl-2-[5-(2-methoxy-ethoxy)-pyridin-2-ylamino]-8H-pyrido[2,3-d]pyrimidin-7-one, 6-Acetyl-8-cyclopentyl-2-[5-(2-methoxy-ethoxymethyl)-pyridin-2-ylamino]-8H-pyrido[2,3-d]pyrimidin-7-one, 6-Acetyl-8-cyclopentyl-2-[5-(2-diethylamino-ethoxy)-pyridin-2-ylamino]-8H-pyrido[2,3-d]pyrimidin-7-one, 6-Acetyl-8-cyclopentyl-2-(5-pyrrolidin-1-yl-pyridin-2-ylamino)-8H-pyrido[2,3-d]pyrimidin-7-one, 6-Acetyl-8-cyclopentyl-2-(6-methyl-5-piperazin-1-yl-pyridin-2-ylamino)-8H-pyrido[2,3-d]pyrimidin-7-one, 6-Bromo-8-cyclopentyl-2-[5-(2-methoxy-ethoxy)-pyridin-2-ylamino]-8H-pyrido[2,3d]pyrimidin-7-one, 6-Bromo-8-cyclopentyl-2-[5-(2-methoxy-ethylamino)-pyridin-2-ylamino]-8H-pyrido[2,3-d]pyrimidin-7-one, 2-(5-Azetidin-1-yl-pyridin-2-ylamino)-6-bromo-8-cyclopentyl-8H-pyrido[2,3-d]pyrimidin-7-one, 2-(5-Azepan-1-yl-pyridin-2-ylamino)-6-bromo-8-cyclopentyl-8H-pyrido[2,3-d]pyrimidin-7-one, N-[6-(6-Bromo-8-cyclopentyl-7-oxo-7,8-dihydro-pyrido[2,3-d]pyrimidin-2-ylamino)-pyridin-3-yl]-acetamide, 6-Bromo-8-cyclopentyl-2-(5-phenylamino-pyridin-2-ylamino)-8H-pyrido[2,3-d]pyrimidin-7-one, 6-Bromo-8-cyclopentyl-2-[5-(4-fluoro-benzylamino)-pyridin-2-ylamino]-8H-pyrido[2,3-d]pyrimidin-7-one, N-[6-(6-Bromo-8-cyclopentyl-7-oxo-7,8-dihydro-pyrido[2,3-d]pyrimidin-2-ylamino)-pyridin-3-yl]-methanesulfonamide, 6-Bromo-8-cyclopentyl-2-(5-methanesulfonyl-pyridin-2-ylamino)-8H-pyrido[2,3-d]pyrimidin-7-one, 6-Bromo-8-cyclopentyl-2-(5-phenyl-pyridin-2-ylamino)-8H-pyrido[2,3-d]pyrimidin-7-one, 6-Amino-8-cyclopentyl-2-[5-(2-methoxy-ethoxy)-pyridin-2-ylamino]-8H-pyrido[2,3-d]pyrimidin-7-one, 6-Amino-8-cyclopentyl-2-[5-(2-methoxy-ethylamino)-pyridin-2-ylamino]-8H-pyrido[2,3-d]pyrimidin-7-one, 6-Amino-2-(5-azetidin-1-yl-pyridin-2-ylamino)-8-cyclopentyl-8H-pyrido[2,3-d]pyrimidin-7-one, 6-Amino-2-(5-azepan-1-yl-pyridin-2-ylamino)-8-cyclopentyl-8H-pyrido[2,3-d]pyrimidin-7-one, N-[6-(6-Amino-8-cyclopentyl-7-oxo-7,8-dihydro-pyrido[2,3-d]pyrimidin-2-ylamino)-pyridin-3-yl]-acetamide, 6-Amino-8-cyclopentyl-2-(5-phenylamino-pyridin-2-ylamino)-8H-pyrido[2,3-d]pyrimidin-7-one, 6-Amino-8-cyclopentyl-2-[5-(4-fluoro-benzylamino)-pyridin-2-ylamino]-8H-pyrido[2,3-d]pyrimidin-7-one, N-[6-(6-Amino-8-cyclopentyl-7-oxo-7,8-dihydro-pyrido[2,3-d]pyrimidin-2-ylamino)-pyridin-3-yl]-methanesulfonamide, 6-Amino-8-cyclopentyl-2-(5-methanesulfonyl-pyridin-2-ylamino)-8H-pyrido[2,3-d]pyrimidin-7-one, 6-Amino-8-cyclopentyl-2-(5-phenyl-pyridin-2-ylamino)-8H-pyrido[2,3-d]pyrimidin-7-one, 6-Acetyl-8-cyclopentyl-2-[5-(2-methoxy-ethoxy)-pyridin-2-ylamino]-5-methyl-8H-pyrido[2,3-d]pyrimidin-7-one, 6-Acetyl-8-cyclopentyl-2-[5-(2-methoxy-ethylamino)-pyridin-2-ylamino]-5-methyl-8H-pyrido[2,3-d]pyrimidin-7-one, 6-Acetyl-2-(5-azetidin-1-yl-pyridin-2-ylamino)-8-cyclopentyl-5-methyl-8H-pyrido[2,3-d]pyrimidin-7-one, 6-Acetyl-2-(5-azepan-1-yl-pyridin-2-ylamino)-8-cyclopentyl-5-methyl-8H-pyrido[2,3-d]pyrimidin-7-one, N-[6-(6-Acetyl-8-cyclopentyl-5-methyl-7-oxo-7,8-dihydro-pyrido[2,3-d]pyrimidin-2-ylamino)-pyridin-3-yl]-acetamide, 6-Acetyl-8-cyclopentyl-5-methyl-2-(5-phenylamino-pyridin-2-ylamino)-8H-pyrido[2,3-d]pyrimidin-7-one, 6-Acetyl-8-cyclopentyl-2-[5-(4-fluoro-benzylamino)-pyridin-2-ylamino]-5-methyl-8H-pyrido[2,3-d]pyrimidin-7-one, N-[6-(6-Acetyl-8-cyclopentyl-5-methyl-7-oxo-7,8-dihydro-pyrido[2,3-d]pyrimidin-2-ylamino)-pyridin-3-yl]-methanesulfonamide, 6-Acetyl-8-cyclopentyl-2-(5-methanesulfonyl-pyridin-2-ylamino)-5-methyl-8H-pyrido[2,3-d]pyrimidin-7-one, 6-Acetyl-8-cyclopentyl-5-methyl-2-(5-phenyl-pyridin-2-ylamino)-8H-pyrido[2,3-d]pyrimidin-7-one, 6-Benzyl-8-cyclopentyl-2-[5-(2-methoxy-ethoxy)-pyridin-2-ylamino]-8H-pyrido[2,3-d]pyrimidin-7-one, 6-Benzyl-8-cyclopentyl-2-[5-(2-methoxy-ethylamino)-pyridin-2-ylamino]-8H-pyrido[2,3-d]pyrimidin-7-one, 2-(5-Azetidin-1-yl-pyridin-27ylamino)-6-benzyl-8-cyclopentyl-8H-pyrido[2,3-d]pyrimidin-7-one, 2-(5-Azepan-1-yl-pyridin-2-ylamino)-6-benzyl-8-cyclopentyl-8H-pyrido[2,3-d]pyrimidin-7-one, N-[6-(6-Benzyl-8-cyclopentyl-7-oxo-7,8-dihydro-pyrido[2,3-d]pyrimidin-2-ylamino)-pyridin-3-yl]-acetamide, 6-Benzyl-8-cyclopentyl-2-(5-phenylamino-pyridin-2-ylamino)-8H-pyrido[2,3-d]pyrimidin-7-one, 6-Benzyl-8-cyclopentyl-2-[5-(4-fluoro-benzylamino)-pyridin-2-ylamino]-8H-pyrido[2,3-d]pyrimidin-7-one, N-[6-(6-Benzyl-8-cyclopentyl-7-oxo-7,8-dihydro-pyrido[2,3-d]pyrimidin-2-ylamino)-pyridin-3-yl]-methanesulfonamide, 6-Benzyl-8-cyclopentyl-2-(5-methanesulfonyl-pyridin-2-ylamino)-8H-pyrido[2,3-d]pyrimidin-7-one, 6-Benzyl-8-cyclopentyl-2-(5-phenyl-pyridin-2-ylamino)-8H-pyrido[2,3-d]pyrimidin-7-one, 8-Cyclopentyl-6-hydroxymethyl-2-[5-(2-methoxy-ethoxy)-pyridin-2-ylamino]-8H-pyrido[2,3-d]pyrimidin-7-one, 8-Cyclopentyl-6-hydroxymethyl-2-[5-(2-methoxy-ethylamino)-pyridin-2-ylamino]-8H-pyrido[2,3-d]pyrimidin-7-one, 2-(5-Azetidin-1-yl-pyridin-2-ylamino)-8-cyclopentyl-6-hydroxymethyl-8H-pyrido[2,3-d]pyrimidin-7-one, 2-(5-Azepan-1-yl-pyridin-2-ylamino)-8-cyclopentyl-6-hydroxymethyl-8H-pyrido[2,3-d]pyrimidin-7-one, N-[6-(8-Cyclopentyl-6-hydroxymethyl-7-oxo-7,8-dihydro-pyrido[2,3-d]pyrimidin-2-ylamino)-pyridin-3-yl]-acetamide, 8-Cyclopentyl-6-hydroxymethyl-2-(5-phenylamino-pyridin-2-ylamino)-8H-pyrido[2,3-d]pyrimidin-7-one, 8-Cyclopentyl-2-[5-(4-fluoro-benzylamino)-pyridin-2-ylamino]-6-hydroxymethyl-8H-pyrido[2,3-d]pyrimidin-7-one, N-[6-(8-Cyclopentyl-6-hydroxymethyl-7-oxo-7,8-dihydro-pyrido[2,3-d]pyrimidin-2-ylamino)-pyridin-3-yl]-methanesulfonamide, 8-Cyclopentyl-6-hydroxymethyl-2-(5-methanesulfonyl-pyridin-2-ylamino)-8H-pyrido[2,3-d]pyrimidin-7-one, 8-Cyclopentyl-6-hydroxymethyl-2-(5-phenyl-pyridin-2-ylamino)-8H-pyrido[2,3-d]pyrimidin-7-one, 8-Cyclopentyl-6-ethyl-2-[5-(2-methoxy-ethoxy)-pyridin-2-ylamino]-8H-pyrido[2,3-d]pyrimidin-7-one, 8-Cyclopentyl-6-ethyl-2-[5-(2-methoxy-ethylamino)-pyridin-2-ylamino]-8H-pyrido[2,3-d]pyrimidin-7-one, 2-(5-Azetidin-1-yl-pyridin-2-ylamino)-8-cyclopentyl-6-ethyl-8H-pyrido[2,3-d]pyrimidin-7-one, 2-(5-Azepan-1-yl-pyridin-2-ylamino)-8-cyclopentyl-6-ethyl-8H-pyrido[2,3-d]pyrimidin-7-one, N-[6-(8-Cyclopentyl-6-ethyl-7-oxo-7,8-dihydro-pyrido[2,3-d]pyrimidin-2-ylamino)-pyridin-3-yl]-acetamide, 8-Cyclopentyl-6-ethyl-2-(5-phenylamino-pyridin-2-ylamino)-8H-pyrido[2,3-d]pyrimidin-7-one, 8-Cyclopentyl-6-ethyl-2-[5-(4-fluoro-benzylamino)-pyridin-2-ylamino]-8H-pyrido[2,3-d]pyrimidin-7-one, N-[6-(8-Cyclopentyl-6-ethyl-7-oxo-7,8-dihydro-pyrido[2,3-d]pyrimidin-2-ylamino)-pyridin-3-yl]-methanesulfonamide, 8-Cyclopentyl-6-ethyl-2-(5-methanesulfonyl-pyridin-2-ylamino)-8H-pyrido[2,3-d]pyrimidin-7-one, 8-Cyclopentyl-6-ethyl-2-(5-phenyl-pyridin-2-ylamino)-8H-pyrido[2,3-d]pyrimidin-7-one, 6-Bromo-8-cyclopentyl-2-[5-(piperazine-1-carbonyl)-pyridin-2-ylamino]-8H-pyrido[2,3-d]pyrimidin-7-one, 6-Bromo-8-cyclopentyl-2-[5-(3,5-dimethyl-piperazine-1-carbonyl)-pyridin-2-ylamino]-8H-pyrido[2,3-d]pyrimidin-7-one, 2-[5-(3-Amino-pyrrolidine-1-carbonyl)-pyridin-2-ylamino]-6-bromo-8-cyclopentyl-8H-pyrido[2,3-d]pyrimidin-7-one, 6-Bromo-8-cyclopentyl-2-[5-(morpholine-4-carbonyl)-pyridin-2-ylamino]-8H-pyrido[2,3-d]pyrimidin-7-one, 6-Bromo-8-cyclopentyl-5-methyl-2-[5-(piperazine-1-carbonyl)-pyridin-2-ylamino]-8H-pyrido[2,3-d]pyrimidin-7-one, 6-Bromo-8-cyclopentyl-2-[5-(3,5-dimethyl-piperazine-1-carbonyl)-pyridin-2-ylamino]-5-methyl-8H-pyrido[2,3-d]pyrimidin-7-one, 2-[5-(3-Amino-pyrrolidine-1-carbonyl)-pyridin-2-ylamino]-6-bromo-8-cyclopentyl-5-methyl-8H-pyrido[2,3-d]pyrimidin-7-one, 6-Bromo-8-cyclopentyl-5-methyl-2-[5-(morpholine-4-carbonyl)-pyridin-2-ylamino]-8H-pyrido[2,3-d]pyrimidin-7-one, 6-Acetyl-8-cyclopentyl-5-methyl-2-[5-(piperazine-1-carbonyl)-pyridin-2-ylamino]-8H-pyrido[2,3-d]pyrimidin-7-one, 6-Acetyl-8-cyclopentyl-2-[5-(3,5-dimethyl-piperazine-1-carbonyl)-pyridin-2-ylamino]-5-methyl-8H-pyrido[2,3-d]pyrimidin-7-one, 6-Acetyl-2-[5-(3-amino-pyrrolidine-1-carbonyl)-pyridin-2-ylamino]-8-cyclopentyl-5-methyl-8H-pyrido[2,3-d]pyrimidin-7-one, 6-Acetyl-8-cyclopentyl-5-methyl-2-[5-(morpholine-4-carbonyl)-pyridin-2-ylamino]-8H-pyrido[2,3-d]pyrimidin-7-one, 8-Cyclopentyl-6-ethyl-2-[5-(piperazine-1-carbonyl)-pyridin-2-ylamino]-8H-pyrido[2,3-d]pyrimidin-7-one, 8-Cyclopentyl-2-[5-(3,5-dimethyl-piperazine-1-carbonyl)-pyridin-2-ylamino]-6-ethyl-8H-pyrido[2,3-d]pyrimidin-7-one, 2-[5-(3-Amino-pyrrolidine-1-carbonyl)-pyridin-2-ylamino]-8-cyclopentyl-6-ethyl-8H-pyrido[2,3-d]pyrimidin-7-one, 8-Cyclopentyl-6-ethyl-2-[5-(morpholine-4-carbonyl)-pyridin-2-ylamino]-8H-pyrido[2,3-d]pyrimidin-7-one, 6-Bromo-8-cyclopentyl-2-[5-(piperazine-1-sulfonyl)-pyridin-2-ylamino]-8H-pyrido[2,3-d]pyrimidin-7-one, 6-Bromo-8-cyclopentyl-2-[5-(morpholine-4-sulfonyl)-pyridin-2-ylamino]-8H-pyrido[2,3-d]pyrimidin-7-one, 2-[5-(3-Amino-pyrrolidine-1-sulfonyl)-pyridin-2-ylamino]-6-bromo-8-cyclopentyl-8H-pyrido[2,3-d]pyrimidin-7-one, 6-Bromo-8-cyclopentyl-2-[5-(3,5-dimethyl-piperazine-1-sulfonyl)-pyridin-2-ylamino]-8H-pyrido[2,3-d]pyrimidin-7-one, 6-Bromo-8-cyclopentyl-5-methyl-2-[5-(piperazine-1-sulfonyl)-pyridin-2-ylamino]-8H-pyrido[2,3-d]pyrimidin-7-one, 6-Bromo-8-cyclopentyl-5-methyl-2-[5-(morpholine-4-sulfonyl)-pyridin-2-ylamino]-8H-pyrido[2,3-d]pyrimidin-7-one, 2-[5-(3-Amino-pyrrolidine-1-sulfonyl)-pyridin-2-ylamino]-6-bromo-8-cyclopentyl-5-methyl-8H-pyrido[2,3-d]pyrimidin-7-one, 6-Bromo-8-cyclopentyl-2-[5-(3,5-dimethyl-piperazine-1-sulfonyl)-pyridin-2-ylamino]-5-methyl-8H-pyrido[2,3-d]pyrimidin-7-one, 8-Cyclopentyl-6-ethyl-2-[5-(piperazine-1-sulfonyl)-pyridin-2-ylamino]-8H-pyrido[2,3-d]pyrimidin-7-one, 8-Cyclopentyl-6-ethyl-2-[5-(morpholine-4-sulfonyl)-pyridin-2-ylamino]-8H-pyrido[2,3-d]pyrimidin-7-one, 2-[5-(3-Amino-pyrrolidine-1-sulfonyl)-pyridin-2-ylamino]-8-cyclopentyl-6-ethyl-8H-pyrido[2,3-d]pyrimidin-7-one, 8-Cyclopentyl-2-[5-(3,5-dimethyl-piperazine-1-sulfonyl)-pyridin-2-ylamino]-6-ethyl-8H-pyrido[2,3-d]pyrimidin-7-one, 6-Acetyl-8-cyclopentyl-5-methyl-2-[5-(piperazine-1-sulfonyl)-pyridin-2-ylamino]-8H-pyrido[2,3-d]pyrimidin-7-one, 6-Acetyl-8-cyclopentyl-5-methyl-2-[5-(morpholine-4-sulfonyl)-pyridin-2-ylamino]-8H-pyrido[2,3-d]pyrimidin-7-one, 6-Acetyl-2-[5-(3-amino-pyrrolidine-1-sulfonyl)-pyridin-2-ylamino]-8-cyclopentyl-5-methyl-8H-pyrido[2,3-d]pyrimidin-7-one, 6-Acetyl-8-cyclopentyl-2-[5-(3,5-dimethyl-piperazine-1-sulfonyl)-pyridin-2-ylamino]-5-methyl-8H-pyrido[2,3-d]pyrimidin-7-one, and 6-Acetyl-8-cyclopentyl-5-methyl-2-([1,6]naphthyridin-2-ylamino)-8H-pyrido[2,3-d]pyrimidin-7-one, 6-Acetyl-8-cyclopentyl-2-[5-(1,1-dioxo-116-thiomorpholin-4-yl)-pyridin-2-ylamino]-5-methyl-8H-pyrido[2,3-d]pyrimidin-7-one, 8-Cyclopentyl-6-hydroxymethyl-5-methyl-2-(5-piperazin-1-yl-pyridin-2-ylamino)-8H-pyrido[2,3-d]pyrimidin-7-one, 6-Acetyl-2-(3-chloro-5-piperazin-1-yl-pyridin-2-ylamino)-8-cyclopentyl-5-methyl-8H-pyrido[2,3-d]pyrimidin-7-one, 4-[6-Acetyl-5-methyl-7-oxo-2-(pyridin-2-ylamino)-7H-pyrido[2,3-d]pyrimidin-8-yl]-cyclohexanecarboxylic acid, 4-[6-Acetyl-2-(5-dimethylamino-pyridin-2-ylamino)-5-methyl-7-oxo-7H-pyrido[2,3-d]pyrimidin-8-yl]-cyclohexanecarboxylic acid, 6-Bromo-8-cyclopentyl-5-methyl-2-[5-(piperazine-1-sulfonyl)-pyridin-2-ylamino]-8H-pyrido[2,3-d]pyrimidin-7-one, 6-(8-Cyclopentyl-6-ethyl-7-oxo-7,8-dihydro-pyrido[2,3-d]pyrimidin-2-ylamino)-3-piperazin-1-yl-pyridine-2-carboxylic acid, 2-(6-Acetyl-5-piperazin-1-yl-pyridin-2-ylamino)-8-cyclopentyl-6-ethyl-8H-pyrido[2,3-d]pyrimidin-7-one, 3-{2-[6-(8-Cyclopentyl-6-ethyl-7-oxo-7,8-dihydro-pyrido[2,3-d]pyrimidin-2-ylamino)-pyridin-3-yloxy]-ethoxy}-propionic acid, [6-(8-Cyclopentyl-6-ethyl-7-oxo-7,8-dihydro-pyrido[2,3-d]pyrimidin-2-ylamino)-pyridin-3-yloxy]-acetic acid, 8-Cyclopentyl-2-(5-{2-[2-(5-methyl-pyridin-2-yl)-ethoxy]-ethoxy}-pyridin-2-ylamino)-8H-pyrido[2,3-d]pyrimidin-7-one, 2-[5-(3-Benzenesulfonyl-propoxy)-pyridin-2-ylamino]-8-cyclopentyl-8H-pyrido[2,3-d]pyrimidin-7-one, 8-Cyclopentyl-6-ethyl-2-{5-[2-(2-methoxy-ethoxy)-ethoxy]-pyridin-2-ylamino}-8H-pyrido[2,3-d]pyrimidin-7-one, 8-Cyclopentyl-2-(5-{[3-(3,5-dimethyl-piperazin-1-yl)-propyl]-methyl-amino}-pyridin-2-ylamino)-8H-pyrido[2,3-d]pyrimidin-7-one, 8-Cyclopentyl-2-{5-[(3-imidazol-1-yl-propyl)-methyl-amino]-pyridin-2-ylamino}-8H-pyrido[2,3-d]pyrimidin-7-one, 6-Acetyl-5-methyl-2-(5-methyl-pyridin-2-ylamino)-8-piperidin-4-yl-8H-pyrido[2,3-d]pyrimidin-7-one, and 6-Acetyl-2-[5-(3,4-dihydroxy-pyrrolidin-1-yl)-pyridin-2-ylamino]-8-methoxymethyl-5-methyl-8H-pyrido[2,3-d]pyrimidin-7-one.

In some embodiments, the compound is selected from a group consisting of: 8-Cyclopentyl-2-(pyridin-2-ylamino)-8H-pyrido[2,3-d]pyrimidin-7-one, 6-Bromo-8-cyclopentyl-2-(5-piperazin-1-yl-pyridin-2-ylamino)-8H-pyrido [2,3-d]pyrimidin-7-one hydrochloride, 8-Cyclopentyl-6-ethyl-2-(5-piperazin-1-yl-pyridin-2-ylamino)-8H-pyrido[2,3-d]pyrimidin-7-one hydrochloride, 8-Cyclopentyl-7-oxo-2-(5-piperazin-1-yl-pyridin-2-ylamino)-7,8-dihydro-pyrido[2,3-d]pyrimidine-6-carboxylic acid ethyl ester hydrochloride, 6-Amino-8-cyclopentyl-2-(5-piperazin-1-yl-pyridin-2-ylamino)-8H-pyrido[2,3-d]pyrimidin-7-one hydrochloride, 6-Bromo-8-cyclopentyl-2-[5-((R)-methy-1-pyrrolidin-2-yl)-pyridin-2-ylamino]-8H-pyrido[2,3-d]pyrimidin-7-one hydrochloride, 6-Bromo-8-cyclohexyl-2-(pyridin-2-yl-amino)-8H-pyrido[2,3-d]pyrimidin-7-one, 6-Bromo-8-cyclopentyl-2-methyl-8H-pyrido[2,3-d]pyrimidin-7-one, 6-Bromo-8-cyclopentyl-5-methyl-2-(5-piperazin-1-yl-pyridin-2-ylamino)-8H-pyrido[2,3-d]pyrimidin-7-one, 8-Cyclopentyl-6-fluoro-2-(5-piperazin-1-yl-pyridin-2-ylamino)-8H-pyrido[2,3-d]pyrimidin-7-one hydrochloride, 8-Cyclopentyl-6-methyl-2-(5-piperazin-1-yl-pyridin-2-ylamino)-8H-pyrido[2,3-d]pyrimidin-7-one hydrochloride, 8-Cyclopentyl-6-isobutoxy-2-(5-piperazin-1-yl-pyridin-2-ylamino)-8H-pyrido[2,3-d]pyrimidin-7-one hydrochloride, 6-Benzyl-8-cyclopentyl-2-(5-piperazin-1-yl-pyridin-2-ylamino)-8H-pyrido[2,3-d]pyrimidin-7-one hydrochloride, 8-Cyclopentyl-6-hydroxymethyl-2-(5-piperazin-1-yl-pyridin-2-ylamino)-8H-pyrido[2,3-d]pyrimidin-7-one hydrochloride, 2-[5-(4-tert-Butoxycarbonyl-piperazin-1-yl)-pyridin-2-ylamino]-8-cyclopentyl-5-methyl-7-oxo-7,8-dihydro-pyrido[2,3-d]pyrimidine-6-carboxylic acid ethyl ester, 6-Acetyl-8-cyclopentyl-2-(5-piperazin-1-yl-pyridin-2-ylamino)-8H-pyrido[2,3-d]pyrimidin-7-one, 6-Acetyl-8-cyclopentyl-5-methyl-2-(5-piperazin-1-yl-pyridin-2-ylamino)-8H-pyrido[2,3-d]pyrimidin-7-one, 6-Bromo-8-cyclopentyl-5-methyl-2-(pyridin-2-ylamino)-8H-pyrido[2,3-d]pyrimidin-7-one, and 6-Bromo-8-cyclopentyl-2-(pyridin-2-ylamino)-8H-pyrido[2,3-d]pyrimidin-7-one.

In some embodiments, the compound is 6-Acetyl-8-cyclopentyl-5-methyl-2-(5-piperazin-1-yl-pyridin-2-ylamino)-8H-pyrido[2,3-d]pyrimidin-7-one.

In some embodiments, the medicinal product further comprises a fulvestrant.

In some embodiments, the HER2 inhibitor and the CDK inhibitor are not mixed with each other in the composition.

In some embodiments, the HER2 inhibitor, the CDK inhibitor and fulvestrant are not mixed with each other in the composition.

In another aspect, the present application provides the HER2 inhibitor of the present application for the use of preventing, alleviating or treating tumor or inhibiting tumor growth in combination with the CDK inhibitor of the present application.

In another aspect, the present application provides a use of the HER2 inhibitor of the present application in combination with the CDK inhibitor of the present application in the preparation of a medicament for preventing, alleviating or treating tumor or inhibiting tumor growth in a subject in need thereof.

In another aspect, the present application provides a method of preventing, alleviating or treating tumor or inhibiting tumor growth in a subject in need thereof, comprising: administrating the HER2 inhibitor of the present application in combination with the CDK inhibitor of the present application to the subject.

In another aspect, the present application provides a method of preventing, alleviating or treating tumor or inhibiting tumor growth in a subject in need thereof, comprising: administrating the composition of the present application to the subject.

In some embodiments, the tumor comprises solid tumor.

In some embodiments, the tumor comprises metastatic tumor, early tumor and/or locally advanced tumor.

In some embodiments, the tumor comprises HER2 positive tumor and/or HER2 low-expression tumor.

In some embodiments, the tumor comprises a breast cancer and/or a gastric cancer.

In some embodiments, the breast cancer comprises HER2 positive breast cancer and/or HER2 low-expression breast cancer.

In some embodiments, the tumor is HR negative or HR positive breast cancer.

In some embodiments, the breast cancer comprises early breast cancer, locally advanced breast cancer and/or metastatic breast cancer; and/or the gastric cancer comprises early gastric cancer, locally advanced gastric cancer and/or metastatic gastric cancer.

In some embodiments, the tumor is selected from the group consisting of cancers of the breast, ovary, cervix, prostate, testis, esophagus, stomach, skin, lung, bone, colon, pancreas, thyroid, biliary passages, buccal cavity and pharynx (oral), lip, tongue, mouth, pharynx, small intestine, colon-rectum, large intestine, rectum, brain and central nervous system, glioblastoma, neuroblastoma, keratoacanthoma, epidermoid carcinoma, large cell carcinoma, adenocarcinoma, adenocarcinoma, adenoma, adenocarcinoma, follicular carcinoma, undifferentiated carcinoma, papillary carcinoma, seminoma, melanoma, Sarcoma, bladder carcinoma, liver carcinoma, kidney carcinoma, myeloid disorders, lymphoid disorders, Hodgkin's, hairy cells, and leukemia.

In some embodiments, the HER2 inhibitor is formulated for intravenous injection.

In some embodiments, the HER2 inhibitor is administrated to the subject in need at a dose of about 1 mg/kg to about 30 mg/kg.

In some embodiments, the HER2 inhibitor is administrated to the subject in need every two weeks.

In some embodiments, the CDK inhibitor is formulated for oral use.

In some embodiments, the CDK inhibitor is administrated to the subject in need at a dose of about 10 mg/kg to about 50 mg/kg.

In some embodiments, the CDK inhibitor is administrated to the subject in need every day for three weeks.

In some embodiments, the method further comprises administering to the subject an effective amount of fulvestrant.

In some embodiments, fulvestrant in the medicinal product is formulated for intramuscular injection.

In some embodiments, fulvestrant in the medicinal product is administrated to the subject in need at a dose of about 400 mg to about 600 mg.

In some embodiments, fulvestrant in the medicinal product is administrated to the subject in need on Days 1 and 15 of Cycle 1, and then on Day 1 of each subsequent 28-day cycle.

In some embodiments, the subject was not responsive to a conventional therapy for HER2-related tumor.

In some embodiments, the conventional therapy for HER2-related tumor comprises administrating HER2-ADC, MBC hormone, Taxane, pyrotinib, neratinib, tucatinib, trastuzumab and/or pertuzumab.

In some embodiments, the conventional therapy for HER2-related tumor comprises administrating docetaxel, capecitabine and/or lapatinib.

Additional aspects and advantages of the present application will become readily apparent to those skilled in this art from the following detailed description, wherein only illustrative embodiments of the present application are shown and described. As will be realized, the present application is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1 illustrates a schematic diagram of study design of the study described in example 1 and 2, wherein HBI represents the HER2 inhibitor of the present application.

FIG. 2 illustrates the tumor volume in HCC1954 XENO model after the treatment of the medicinal product of present application, wherein HBI represents the HER2 inhibitor of the present application.

FIG. 3 illustrates ER, PR, EGFR and HER2 biomarkers in HCC1954 XENO model after the treatment of the medicinal product of present application.

FIG. 4 illustrates the tumor volume in MCF-7 XENO model after the treatment of the medicinal product of present application, wherein HBI represents the HER2 inhibitor of the present application.

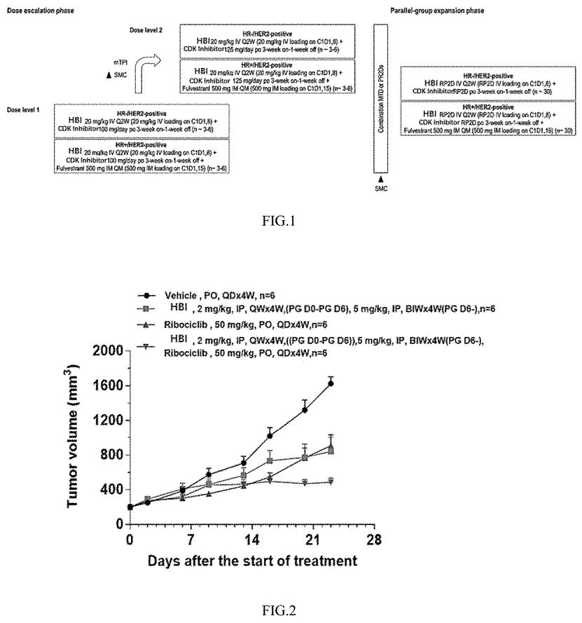

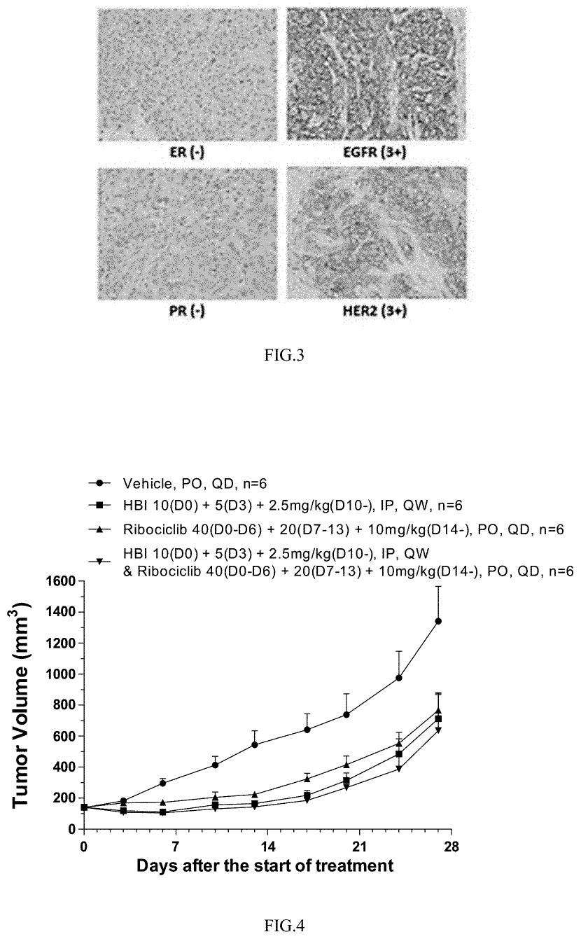

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

In the present application, the term “HER2” as used herein, generally refers to the type I transmembrane protein, also known as c-erbB2, ErbB2 or Neu, belonging to the family of epidermal growth factor receptors. In the context of the present application, the term “HER2” also encompasses homologues, variants and isoforms, including splice isoforms, of HER2. The term “HER2” further encompasses proteins having the sequence of one or more of a HER2 homologue, variant and isoform, as well as fragments of the sequences, provided that the variant proteins (including isoforms), homologous proteins and/or fragments are recognized by one or more HER2 specific antibodies, such as provided as Pertuzumab, Trastuzumab and Margetuximab. The HER2 may be a human HER2. The human HER2 gene is mapped to chromosomal location 17q12, and the genomic sequence of HER2 gene can be found in GenBank at NG_007503.1. In human, there are five HER2 isoforms: A, B, C, D, and E; the term “HER2” is used herein to refer collectively to all HER2 isoforms.

In the present application, the term “antibody” as used herein, generally refers to an immunoglobulin or a fragment or a derivative thereof, and encompasses any polypeptide comprising an antigen-binding site, regardless whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)2, Fv, scFv, Fd, dAb, and other antibody fragments that retain antigen-binding function, i.e., the ability to bind, for example, HER2 specifically. Typically, such fragments would comprise an antigen-binding domain.

In the present application, the term “antigen binding portion” of the antibody refers to one or more portions of a full-length antibody, the antigen binding portion maintains the ability of binding to an antigen (such as HER2) that is the same as that bound by the antibody, and competes with the full-length antibody for the specific binding to an antigen. General reference is made to Fundamental Immunology, Ch. 7 (Paul, W., ed., edition II, Raven Press, N.Y. (1989), which is incorporated herein by reference in its entirety and for all purposes. The antigen binding portion can be produced using a recombinant DNA technology or through enzymatic or chemical breakage of a full-length antibody. In some cases, the antigen binding portion comprises a polypeptide such as a Fab, a Fab′, a F(ab′)2, a Fd, a Fv, a dAb, a complementary determining region (CDR) fragment, a single-chain antibody (such as a scFv), a chimeric antibody, and a diabody, and it comprises at least the part of the antibody sufficiently endowing the polypeptide with the specific antigen binding ability. The antigen binding portion (such as the above antibody fragment) of the antibody may be obtained from a given antibody using a conventional technology (such as recombinant DNA technology or enzymatic or chemical breakage process) known to those skilled in the art, and are screened for its specificity in a process that is the same for screening full-length antibodies.

In the present application, the term “variable region” or “variable domain” of an antibody, as used herein, generally refers to the amino-terminal domains of the heavy or light chain of an antibody. The variable domains of the heavy chain and light chain may be referred to as “VH” and “VL”, respectively. These domains are generally the most variable parts of the antibody (relative to other antibodies of the same class) and contain the antigen binding sites.

In the present application, the term “bispecific antibody” refers to an antibody that can respectively bind with two different antigens or the antigen epitopes thereof. For example, the bispecific antibody may comprise at least one kind of light chain or the fragment thereof, as well as at least one kind of heavy chain or the fragment thereof. For example, the bispecific antibody may comprise one light chain or the fragment thereof, which may specifically bind to both a first antigen or the antigen epitope thereof and a second antigen or the antigen epitope thereof. For example, the bispecific antibody may comprise two heavy chains or the fragment thereof, which bind to the first antigen or the antigen epitope thereof and the second antigen or the antigen epitope thereof, respectively. For example, the first antigen or the antigen epitope thereof and the second antigen or the antigen epitope thereof may be two different HER2 antigen.

In the present application, the term “antigen” as used herein, generally refers to a structure specifically bound by antibodies and/or a B cell antigen receptor. An antigen may induce the immune system response by interacting with an antibody that matches the antigen's molecular structure. In one embodiment, an antigen may be HER2. For example, the extracellular domain of HER2 (e.g. ECD2 and/or ECD4). The epitope of the pertuzumab may refer to Discovery of epitopes for targeting the human epidermal growth factor receptor 2 (HER2) with antibodies, Mol Oncol. 2009 June; 3(3): 238-247; The epitope of the trastuzumab may refer to Matching of trastuzumab (Herceptin) epitope mimics onto the surface of Her-2/neu—a new method of epitope definition, Mol Immunol. 2005 May; 42(9):1121-4.

In the present application, the term “CDK inhibitor” as used herein, generally refers to an inhibitor of the cyclin-dependent kinase (CDK). The CDK may be involved in regulating transcription, mRNA processing, and the differentiation of nerve cells. Among various CDKs, CDK4 and/or CDK6 has many inhibitors, such as Palbociclib (against CDK4 and CDK6), Flavopiridol (alvocidib) (against CDK 1, 2, 4, 6, 7 and 9), Aminothiazoles (against CDK4), Benzocarbazoles (against CDK4) and Pyrimidines (against CDK4).

In the present application, the term “Ribociclib” as used herein, generally refers to is an inhibitor of cyclin D1/CDK4 and CDK6, and is used for the treatment of certain kinds of breast cancer. It was developed by Novartis and Astex Pharmaceuticals, and the trade name is Kisqali. Ribociclib has the following structure (C₂₃H₃₀N₈O), and its CAS number is 1211441-98-3.

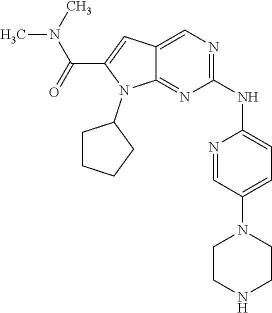 [SMILES: CN(C)C(=O)c1cc2cnc(Nc3ccc(N4CCNCC4)cn3)nc2n1C1CCCC1]

In the present application, the term “Abemaciclib” as used herein, generally refers to a CDK inhibitor selective for CDK4 and CDK6, and is used for the treatment of advanced or metastatic breast cancers. It was developed by Eli Lilly with the trade name Verzenio or Verzenios. Abemaciclib has the following structure (C₂₇H₃₂F₂N₈), and its CAS number is 1231929-97-7.

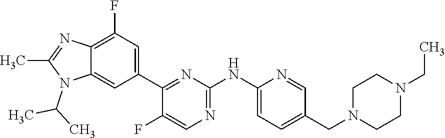 [SMILES: CCN1CCN(Cc2ccc(Nc3ncc(F)c(-c4cc(F)c5nc(C)n(C(C)C)c5c4)n3)nc2)CC1]

In the present application, the term “Palbociclib” as used herein, generally refers to an orally active pyridopyrimidine, first-in-class compound that is a potent and highly selective reversible inhibitor of Cyclin-Dependent Kinase (CDK)4/6. Palbociclib is also called Ibrance, Palbonix or PD-0332991. The compound prevents cellular Deoxyribonucleic Acid (DNA) synthesis by prohibiting progression of the cell cycle from G1 into the S phase. Palbociclib has the following structure (C₂₄H₂₉N₇O₂), and its CAS number is 571190-30-2. It was originally FDA approved on March 2015 for the treatment of HR-positive, HER2-negative advanced or metastatic breast cancer.

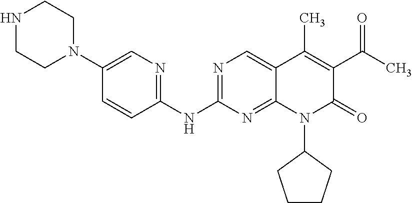 [SMILES: CC(=O)c1c(C)c2cnc(Nc3ccc(N4CCNCC4)cn3)nc2n(C2CCCC2)c1=O]

In the present application, the term “fulvestrant” as used herein, generally refers to a potent anti-estrogen drug that binds and degrades endocrine receptor (ER). Fulvestrant is currently indicated for the treatment of postmenopausal women with metastatic HR positive breast cancer. Fulvestrant has the following structure (C₃₂H₄₇F₅O₃S), and its CAS number is 1245-61-8.

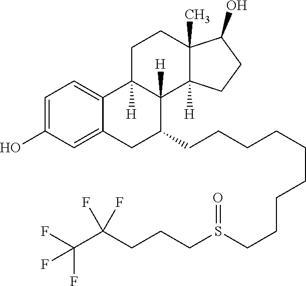

In the present application, the term “breast cancer” as used herein, generally refers to a cancer that develops from breast tissue. Breast cancer cells may or may not have three important receptors: estrogen receptor (ER), progesterone receptor (PR), and HER2.

In the present application, the term “HER2-positive” or “HER2-positive” as used herein, generally refers to a tumor comprising cells which have HER2 protein present at their cell surface. HER2 protein may be overexpressed, e.g., by gene amplification. the solid tumor overexpressing HER2 may be rated by immunohistochemical scores according to the number of copies of HER2 molecules expressed per cell, and can been determined biochemically (refers to Hudziak et al., Proc. Natl. Acad. Sci. USA 84: 7159-7163 [1987]). For example, the HER2-positive solid tumor may comprise a HER-2 positive breast cancer. the HER-2 positive breast cancer may test positive for estrogen receptor and may be a HER2 nonamplified invasive breast cancer. the HER2-positive breast cancer may be advanced. the HER2-positive breast cancer may be metastatic.

In the present application, the term “HER2 low-expression” refers to a tumor comprising cells which expresses very low level of HER2. HER2 low-expression may refer to HER2-negative tumors that test IHC 1⁺ or 2⁺ and FISH⁻. The expression level of HER2 may be measured by immunohistochemistry or FISH. For example, the group with low levels of HER2 may be more likely to be of higher grade, EGFR-positive and ER/HER3/HER4-negative.

In the present application, the term “solid tumor” refers to an abnormal mass of tissue that usually does not contain liquid areas. the solid tumor may be malignant, and may belong to cancer. Different types of solid tumors are named for the type of cells that form them. For example, the solid tumor may comprise breast cancer.

In the present application, the term “metastatic” refers to a tumor that spreads from its site of origin to another part of the body. For many types of tumor, it may be also called stage IV (4) tumor. The metastatic tumor may develop when the tumor cells break away from the main tumor and enter the bloodstream or lymphatic system. For example, breast cancer that spreads to the lung may be called metastatic breast cancer.

In the present application, the term “early tumor” refers to a tumor that has not grown deeply into nearby tissues. The early tumor may be called early-stage cancer, and/or may be called stage I (1) tumor. The early tumor may have not been spread to distant regions.

In the present application, the term “locally advanced tumor” refers to a tumor having grown outside the body part it started in but has not yet spread to other parts of the body. For example, locally advanced breast cancer may be a subset of breast cancer characterized by the most advanced breast tumors in the absence of distant metastasis.

In the present application, the term “treating” as used herein, generally refers to clinical intervention in an attempt to alter the natural course of the individual being treated, and may be performed either for prophylaxis or during the course of clinical pathology. Desirable effects of treatment may also comprise preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. For example, the HER2 inhibitor may be used to delay development of a disease or to slow the progression of a disease.

In the present application, the term “preventing” as used herein, generally refers to delaying the onset, hindering the progress, hindering the appearance, protection against, inhibiting or eliminating the emergence, or reducing the incidence, of such damages, effects or symptoms of a disease or disorder.

In the present application, the term “alleviating” as used herein, generally refers to a process by which the severity of a sign or symptom of a disorder is decreased. the alleviating may comprise alleviating but not eliminating a sign or symptom of a disease or disorder.

In the present application, the term “subject” as used herein, generally refers to an animal, for example, a human. For example, the subject may comprise “non-human animals”, which may comprise mammals such as rats, mice, rabbits, sheep, cats, dogs, cows, pigs, and non-human primates.

In the present application, the term “conventional therapy for HER2-related tumor” as used herein, generally refers to administrating any substances or drugs which block the growth of HER2-related tumor. The conventional therapy for HER2-related tumor may interfere the function of specific molecules responsible for HER2-related (for example, HER2-positive and/or HER2 low expression) tumor cell proliferation and survival. The conventional therapy for HER2-related tumor may comprise any approved drugs specific for treating HER2-related tumor (for example, the HER2-related tumor may be a solid tumor, for example, the HER2-related tumor may at any stage). The conventional therapy for HER2-related tumor may comprise a first-line and/or a second-line approved drug for treating HER2-related tumor (for example, may have been approved for treating a HER2-positive breast cancer). The conventional therapy for HER2-related tumor may comprise any approved drugs suitable for treating HER2-related tumor, including the drugs for universal tumor treatment, for example, a chemotherapy.

In the present application, the term “HR” as used herein, generally refers to hormone receptor. In breast cancer, HR positive (HR⁺) may represents expressing hormone receptor, while HR negative (HR⁻) may represents not expressing hormone receptor. The hormone receptor may comprise estrogen receptors (ER) and progesterone receptors (PR).

In the present application, the term “trastuzumab” as used herein, generally refers to a whole human HER2 monoclonal antibody used to treat breast cancer and stomach cancer. Its brand name can be Herceptin, Herzuma or Ogivri. Trastuzumab may be specifically used for cancer that is HER2 receptor positive.

In the present application, the term “MBC hormone” as used herein, generally refers to hormone therapy for treating breast cancer. In some embodiments, the hormone therapy may stop hormones (for example estrogen, or progesterone) from attaching to the receptors in breast cancer cells. For example, the hormone therapy may comprise administrating Tamoxifen and/or Toremifene.

In the present application, the term “Taxane” as used herein, generally refers to a class of diterpenes. The Taxane may also be used to treat metastatic breast cancer. The CAS number of Taxane may be 1605-68-1. Taxane may have the following formula:

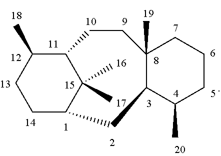

In the present application, the term “HER2-ADC” as used herein, generally refers to a HER2 targeting antibody drug conjugate and is capable of binding to HER2 on the surface of tumor cell. For example, the HER2-ADC may comprise a trastuzumab emtansine (T-DM1), which may be indicated for treatment of HER2-positive metastatic breast cancer. For example, the HER2-ADC may comprise a Trastuzumab deruxtecan (DS-8201a), which may be indicated for treatment of adult patients with unresectable or metastatic HER2-positive breast cancer. For example, the HER2-ADC may comprise a SYD985, in which trastuzumab is linked to the duocarmycin prodrug seco-duocarmycin-hydroxybenzamide-azaindole orseco-DUBA via a cleavable linker.

In the present application, the term “pyrotinib” as used herein, generally refers to an irreversible dual pan-ErbB receptor tyrosine kinase inhibitor. The pyrotinib may target EGFR, HER2, and HER4. The pyrotinib may be used for the treatment of HER2-positive advanced solid tumours. Pyrotinib Racemate is the racemate of Pyrotinib, and Pyrotinib Racemate is a compound having the following formula:

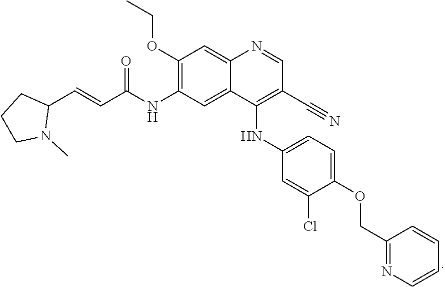 [SMILES: CCOc1cc2ncc(C#N)c(Nc3ccc(OCc4ccccn4)c(Cl)c3)c2cc1NC(=O)/C=C/C1CCCN1C]

In the present application, the term “neratinib” as used herein, generally refers to a tyrosine kinase inhibitor. Neratinib may be used for extended adjuvant treatment of adults with early stage hormone receptor positive HER2-overexpressed/amplified breast cancer. Neratinib is a compound having the following formula:

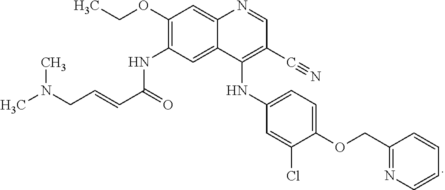 [SMILES: CCOc1cc2ncc(C#N)c(Nc3ccc(OCc4ccccn4)c(Cl)c3)c2cc1NC(=O)/C=C/CN(C)C]

In the present application, the term “tucatinib” as used herein, generally refers to a small molecule inhibitor of HER2. Tucatinib may be used for advanced unresectable or metastatic HER2-positive breast cancer. Tucatinib is a compound having the following formula:

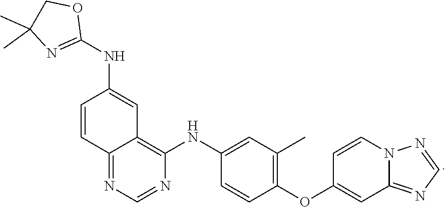 [SMILES: Cc1cc(Nc2ncnc3ccc(NC4=NC(C)(C)CO4)cc23)ccc1Oc1ccn2ncnc2c1]

In the present application, the term “pertuzumab” as used herein, generally refers to a monoclonal antibody used for treating HER2-positive breast cancer. The amino acid sequences of the variable light and variable heavy chains of the pertuzumab (OMNITARG©) may be referred to WO2006033700A2.

In the present application, the term “trastuzumab” as used herein, generally refers to a monoclonal antibody that interferes with the HER2/neu receptor (tradenames Herclon, Herceptin) (Hudis, 2007, N. Engl. J. Med. 3577(1):39-51).

In the present application, the term “docetaxel” as used herein, generally refers to the active ingredient of TAXOTERE© or else TAXOTERE© itself. Docetaxel is a compound having the following formula:

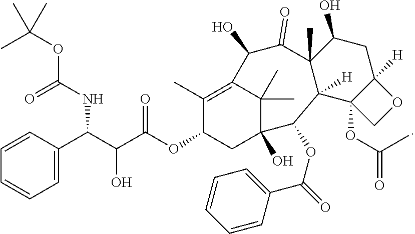

In the present application, the term “capecitabine” as used herein, generally refers to a chemotherapeutic agent that is a prodrug that is converted into 5-FU in the tissues. The chemical name of the capecitabine is pentyl [1-(3,4-dihydroxy-5-methyltetrahydrofuran-2-yl)-5-fluoro-2-oxo-1H-pyrimidin-4-yl]carbamates.

In the present application, the term “lapatinib” as used herein, generally refers to an orally active drug for breast cancer and other solid tumors. It is a dual tyrosine kinase inhibitor which interrupts the HER2/neu and epidermal growth factor receptor (EGFR) pathways. It acts as a dual reversible TKI for both these receptors, thus blocking the downstream MAPK/Erk1/2 and PI3K/AKT pathways. Lapatinib is a compound having the following formula.

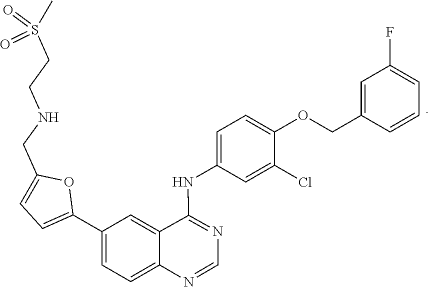 [SMILES: CS(=O)(=O)CCNCc1ccc(-c2ccc3ncnc(Nc4ccc(OCc5cccc(F)c5)c(Cl)c4)c3c2)o1]

In the present application, the term “an effective amount” of a compound of the present application, generally refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc.

In the present application, the term “a” as used herein, generally not means to limit as a singular. In certain embodiments, the term “a” may refer to a plural form. As used throughout this disclosure, the singular forms “a,” “an,” and “the” include plural reference unless the context clearly dictates otherwise.

In the present application, the term “about” as used herein, generally refers to a variation that is within a range of normal tolerance in the art, and generally means within ±10%, such as within 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### Medical Product

In one aspect, the present application provides a medicinal product comprising: a HER2 inhibitor and a CDK inhibitor, wherein said HER2 inhibitor is capable of binding to a first HER2 antigen and a second HER2 antigen, and wherein said CDK inhibitor inhibits CDK4 and/or CDK6.

For example, the medicinal product may be a composition.

For example, the HER2 may be human HER2.

For example, the first HER2 antigen may comprise an epitope within HER2 extracellular domain 2 (ECD2).

For example, the second HER2 antigen may comprise an epitope within HER2 extracellular domain 2 (ECD2).

For example, the second HER2 antigen may comprise an epitope within HER2 extracellular domain 4 (ECD4).

For example, the HER2 inhibitor may be a bispecific antibody or an antigen binding portion thereof.

For example, the antigen binding portion thereof may comprise Fab, Fab′, F(ab)₂, Fv fragment, F(ab′)₂, scFv, di-scFv and/or dAb.

For example, the HER2 inhibitor may be a bispecific antibody or the antigen binding portion thereof, and the bispecific antibody or the antigen binding portion thereof may have a first light chain and a second light chain, and wherein the first light chain and the second light chain comprises a same amino acid sequence.

For example, the HER2 inhibitor may be a bispecific antibody or the antigen binding portion thereof, and the bispecific antibody or the antigen binding portion thereof may have a first heavy chain and a second heavy chain, and wherein the first heavy chain and the second heavy chain are capable of correctly assembling with the light chains respectively under physiological conditions or during in vitro protein expression.

For example, the HER2 inhibitor may be a bispecific antibody or the antigen binding portion thereof, and the bispecific antibody or the antigen binding portion thereof may have a first light chain and a second light chain, and the first light chain and the second light chain may comprise a same amino acid sequence. For example, as the first light chain and the second light chain comprises the same amino acid sequence, the bispecific antibody or the antigen binding portion thereof may have a common light chain.

For example, the common light chain may be obtained via engineering from two different original monoclonal antibodies which may capable of binding to different epitopes of human HER2, respectively. In some cases, the common light chain may be originated from the light chain of either of the two original monoclonal antibodies. In some cases, the common light chain may be modified on the basis of the light chain of either of the two original monoclonal antibodies.

For example, the modification may comprise an insertion, a deletion and/or a substitution in at least one amino acid positions of an amino acid sequence of the light chain of either of the two original monoclonal antibodies. In some cases, the purpose of the modification is to maintain the affinity between the bispecific antibody or the antigen binding portion thereof to the corresponding epitopes.

In present application, light chain constant regions of the bispecific antibody or the antigen binding portion thereof may be of κ type or λ type; the κ-type light chain constant region may comprise various allotypes, such as Km1, Km2 and Km3; the λ-type light chain constant region may comprise various allotypes, such as CL1, CL2, CL3, CL6 and CL7.

In present application, the HER2 inhibitor may be a bispecific antibody or the antigen binding portion thereof, and the bispecific antibody or the antigen binding portion thereof may have a first heavy chain and a second heavy chain.

In present application, the first heavy chain and the second heavy chain are capable of correctly assembling with the light chains respectively under physiological conditions or during in vitro protein expression.

For example, the first light chain and the second light chain may be capable of assembling with a heavy chain of Pertuzumab and a heavy chain of Trastuzumab, respectively.

For example, variable region of the first light chain and/or the second light chain may comprise an amino acid sequence as set forth in SEQ ID NO: 1.

For example, the first light chain and the second light chain may be selected from a light chain of Pertuzumab or a mutant thereof, a light chain of Trastuzumab or a mutant thereof, respectively.

For example, variable region of the first light chain and variable region of the second light chain may be the variable region of light chain of Trastuzumab. For example, the first light chain and the second light chain may be the light chain of Trastuzumab.

For example, the first light chain and the second light chain may comprise an amino acid sequence as set forth in any one of SEQ ID NO: 7-12. For example, the first light chain and the second light chain may comprise an amino acid sequence as set forth in SEQ ID NO: 7.

For example, variable region of the first heavy chain may be a heavy chain variable region of Pertuzumab, and variable region of the second heavy chain may be a heavy chain variable region of Trastuzumab. For example, variable region of the first heavy chain may comprise an amino acid sequence as set forth in SEQ ID NO: 13; and variable region of the second heavy chain may comprise an amino acid sequence as set forth in SEQ ID NO: 14.

In the present application, the first heavy chain and/or the second heavy chain may comprise a constant region. For example, the constant region may be originated from human IgG constant region. For example, heavy chain constant region of the first heavy chain and heavy chain constant region of the second heavy chain may be identical to or different from each other. In some cases, the amino acid sequences of the variable region and the CH1 domain of the first heavy chain and the second heavy chain are identical to those of the original monoclonal antibodies.

In the present application, the bispecific antibody or the antigen binding portion thereof may block both ligand-dependent and ligand-independent HER2 signaling pathway. For example, the IgG1 Fc fragment of the bispecific antibody or the antigen binding portion thereof may bind to FcRγIIIa and may mediate potent ADCC effect. For example, the bispecific antibody or the antigen binding portion thereof may enhance a HER2 internalization and/or may show better anti-tumor activity in preclinical models than using the original monoclonal antibodies alone, e.g. trastuzumab and pertuzumab.

In some cases, the light chain constant region and/or the heavy chain constant region of the bispecific antibody or the antigen binding portion thereof may comprise a modification in order to obtain a better ADCC, CDC, endocytosis, stability, immunogenicity and/or half-life; furthermore, the modification may also facilitate formation of the heterodimer protein during antibody expression. In the present application, technologies for modifying an Fc fragment of the heavy chain are known in the art.

For example, Fc fragment of the first heavy chain may comprise an amino acid sequence as set forth in any one of SEQ ID NO: 19-49, 51-52; and Fc fragment of the second heavy chain may comprise an amino acid sequence as set forth in SEQ ID NO: 19-49, 51-52.

For example, Fc fragment of the first heavy chain may comprise an amino acid sequence as set forth in SEQ ID NO: 19; and Fc fragment of the second heavy chain may comprise an amino acid sequence as set forth in SEQ ID NO: 20.

For example, Fc fragment of the first heavy chain may comprise an amino acid sequence as set forth in SEQ ID NO: 51; and Fc fragment of the second heavy chain may comprise an amino acid sequence as set forth in SEQ ID NO: 52.

For example, the first heavy chain may comprise an amino acid sequence as set forth in SEQ ID NO: 17; and the second heavy chain may comprise an amino acid sequence as set forth in SEQ ID NO: 18.

For example, the first heavy chain may comprise an amino acid sequence as set forth in SEQ ID NO: 15; and the second heavy chain may comprise an amino acid sequence as set forth in SEQ ID NO: 16.

In present application, the HER2 inhibitor may comprise a first light chain, a second light chain, a first heavy chain and a second heavy chain, variable region of the first light chain and/or the second light chain may comprise a sequence as set forth in SEQ ID NO: 1; the variable region of the first heavy chain may comprise an amino acid sequence as set forth in SEQ ID NO: 13; and variable region of the second heavy chain may comprise an amino acid sequence as set forth in SEQ ID NO: 14. The first heavy chain may comprise an amino acid sequence as set forth in SEQ ID NO: 15; and the second heavy chain may comprise an amino acid sequence as set forth in SEQ ID NO: 16.

The amino acid sequence in the present application may also comprise an amino acid sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100%) identity to an amino acid sequence as set forth in any one of SEQ ID NO: 1-104 in the sequence listing. For example, the amino acid sequence in the present application may comprise an amino acid sequence having one or more (e.g., 1-2, 1-3, 1-4, 1-5, 1-6, 1-7, 1-8, 1-9, 1-10, or more) amino acid deletion, insertion and/or substitution in the amino acid sequence as set forth in any one of SEQ ID NO: 1-104 in the sequence listing.

In present application, the HER2 inhibitor may be any present the HER2 inhibitor which may be able to bind to at least two different epitopes of HER2. For example, the HER2 inhibitor may be an the HER2 inhibitor recorded in WO2015077891A1. In some cases, the HER2 inhibitor may be the variant 5019, variant 7091 and variant 10000 recorded in WO2015077891A1. For example, the HER2 inhibitor may be an the HER2 inhibitor recorded in U.S. Ser. No. 10/000,576B1.

For example, the HER2 inhibitor may be an the HER2 inhibitor recorded in WO2015091738A1, in WO2015157592A1, in WO2015077891A1 and in WO2018014864A1.

In the present application, the HER2 inhibitor may block both ligand-dependent and ligand-independent HER2 signaling pathway. For example, the IgG1 Fc fragment of the HER2 inhibitor may bind FcRγIIIa and may mediate potent ADCC effect. For example, the HER2 inhibitor may enhance HER2 internalization and has shown better anti-tumor activity in preclinical models than either trastuzumab or pertuzumab used alone.

In the present application, the CDK4 may be human CDK4, and/or the CDK6 may be human CDK6.

In the present application, the CDK inhibitor may further comprise Palbociclib (PD-0332991), Dinaciclib, P276-00, Ronaciclib (BAY 1000394), P1446A-05, AT7519M, SNS-032, SCH 727965 and AG-024322. AG-024322 represents N-[[5-[(3E)-3-(4,6-difluorobenzimidazol-2-ylidene)-1,2-dihydroindazol-5-yl]-4-methylpyridin-3-yl]methyl]ethanamine; AT7519M represents 4-[(2,6-dichlorobenzoyl)amino]-N-piperidin-4-yl-1H-pyrazole-5-carboxamide;methanesulfonic acid; P1446A-05 represents voruciclib, 2-(2-chloro-4-(trifluoromethyl)phenyl)-5,7-dihydroxy-8-((2R,3S)-2-(hydroxymethyl)-1-methylpyrrolidin-3-yl)-4H-chromen-4-one; P276-00 represents 2-(2-chlorophenyl)-5,7-dihydroxy-8-[(2R,3S)-2-(hydroxymethyl)-1-methylpyrrolidin-3-yl]chromen-4-one; SCH 727965 represents dinaciclib, 2-[(2S)-1-[3-ethyl-7-[(1-oxidopyridin-1-ium-3-yl)methylamino]pyrazolo[1,5-a]pyrimidin-5-yl]piperidin-2-yl]ethanol; SNS-032 represents N-[5-[(5-tert-butyl-1,3-oxazol-2-yl)methylsulfanyl]-1,3-thiazol-2-yl]piperidine-4-carboxamide.

For example, the CDK inhibitor may be recorded in table 1 of Cyclin-Dependent Kinase Inhibitors as Anticancer Therapeutics, Mol Pharmacol. 2015 November; 88(5): 846-852.

In the present application, the CDK inhibitor may further comprise SHR6390, Pirostini, D-0316, BPI-13650, TQ05510, FCN-437c, HEC80797 and BEBT-209.

For example, the CDK inhibitor may have a structure of formula I.

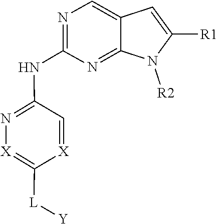

wherein X is CR⁹, or N; R¹ is C₁₋₈alkyl, CN, C(O)OR⁴ or CONR⁵R⁶, a 5-14 membered heteroaryl group, or a 3-14 membered cycloheteroalkyl group; R² is C₁₋₈alkyl, C₃_i₄cycloalkyl, or a 5-14 membered heteroaryl group, and wherein R² may be substituted with one or more C₁₋₈alkyl, or OH; L is a bond, C₁₋₈alkylene, C(O), or C(O)NR¹⁰, and wherein L may be substituted or unsubstituted; Y is H, R¹¹, NR¹²R¹³, OH, or Y is part of the following group

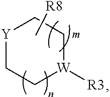

where Y is CR⁹ or N; where 0-3 R⁸ may be present, and R⁸ is C₁₋₈alkyl, oxo, halogen, or two or more R⁸ may form a bridged alkyl group; W is CR⁹, or N; R³ is H, C₁₋₈alkyl, C₁₋₈alkylR¹⁴, C₃-i₄cycloalkyl, C(O)C₁₋₈ alkyl, C₁₋₈haloalkyl, C₁₋₈alkylOH, C(O)NR¹⁴R¹⁵, Ci-gcyanoalkyl, C(O)R¹⁴, Co-₈alkylC(O)C₀₋₈alkylNR¹⁴R¹⁵, C₀₋₈alkylC(O)OR¹⁴, NR¹⁴R¹⁵, SO₂C₁₋₈alkyl, C₁₋₈alkylC₃-i₄cycloalkyl, C(O)C₁₋₈alkylC₃-i₄cycloalkyl, C₁₋₈alkoxy, or OH which may be substituted or unsubstituted when R³ is not H. R⁹ is H or halogen; R⁴, R⁵, R⁶, R⁷, R¹⁰, R¹¹, R¹², R¹³, R¹⁴, and R¹⁵ are each independently selected from H, C₁₋₈alkyl, C₃—H cycloalkyl, a 3-14 membered cycloheteroalkyl group, a Cβ-α aryl group, a 5-14 membered heteroaryl group, alkoxy, C(O)H, C(N)OH, C(N)OCH₃, C(O)C₁₋₃alkyl, C₁₋₈alkylNH₂, C₁₋₆ alkylOH, and wherein R⁴, R⁵, R⁶, R⁷, R¹⁰, R¹¹, R¹², and R¹³, R¹⁴, and R¹⁵ when not H may be substituted or unsubstituted; m and n are independently 0-2; and wherein L, R³, R⁴, R⁵, R⁶, R⁷, R¹⁰, R¹¹, R¹², and R¹³, R¹⁴, and R¹⁵ may be substituted with one or more of C₁₋₈alkyl, C₂₋₈alkenyl, C₂₋₈alkynyl, C3₋₁₄cycloalkyl, 5-14 membered heteroaryl group, C₆₋₁₄aryl group, a 3-14 membered cycloheteroalkyl group, OH, (O), CN, alkoxy, halogen, OrNH₂.

For example, R may be H, C₁₋₈alkyl, C₃_i₄cycloalkyl, C(O)Ci-S alkyl, Ci-₈alkylOH, Ci-gcyanoalkyl, Co-₈alkylC(O)C₀-8alkylNR¹⁴R¹⁵, C₀₋₈alkylC(O)OR¹⁴, NR¹⁴R¹⁵, C₁₋₈alkylC₃_i₄cycloalkyl, C(O)C₁₋₈alkylC_{3_14}cycloalkyl, C₀₋₈alkoxy, C₁₋₈alkylR¹⁴, C₁₋₈haloalkyl, or C(O)R, which may be substituted with one or more of OH, CN, F, or NH₂, and wherein R and R are each independently selected from H, C₁₋₈alkyl, C₃_i₄cycloalkyl, alkoxy, C(O)C_{1_3}alkyl, C_{1_8}alkylNH₂, or C_{1_6}alkylOH.

For example, R may be H, C₁₋₈alkyl, C₃_i₄cycloalkyl, C(O)Ci-S alkyl, Ci-₈alkylOH, Ci-gcyanoalkyl, Co-₈alkylC(O)C₀-8alkylNR¹⁴R¹⁵, C₀₋₈alkylC(O)OR¹⁴, NR¹⁴R¹⁵, C₁₋₈alkylC₃_i₄cycloalkyl, C(O)C₁₋₈alkylC_{3_14}cycloalkyl, C₀₋₈alkoxy, C₁₋₈alkylR¹⁴, C₁₋₈haloalkyl, or C(O)R, which may be substituted with one or more of OH, CN, F, or NH₂, and wherein R and R are each independently selected from H, C₁₋₈alkyl, C₃_i₄cycloalkyl, alkoxy, C(O)C_{1_3}alkyl, C_{1_8}alkylNH₂, or C_{1_6}alkylOH.

For example, Y may be H, OH, or Y may be part of the following group

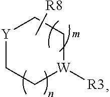

where Y is N and W is CR⁹, or N; where 0-2 R may be present, and R is C₁₋₈alkyl, oxo, or two or more R may form a bridged alkyl group.

For example, L may be a bond, Ci-salkylene, or C(O)NH, or C(O).

For example, R² may be C_{3_H}cycloalkyl.

For example, R may be cyclopentane.

For example, R¹ may be CN, C(O)OR⁴, CONR⁵R, or a 5-14 membered heteroaryl group.

For example, R may be CONR R, a and R⁵ and R⁶ are C₁₋₈alkyl.

For example, X may be CR.

For example, one X may be N and the other X is CR.

For example, X may be CR and Y is

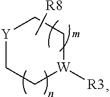

where m and n are 1, and Y and W are N.

For example, the CDK inhibitor may have a structure of formula Ia:

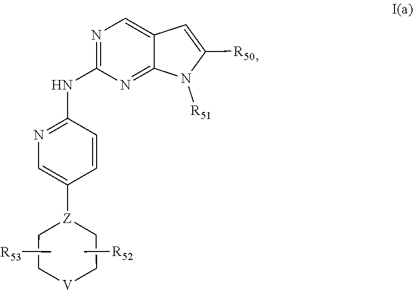

R⁵⁰ is CONR⁵⁴R⁵⁵, or CN; R⁵¹ is C_{3-H}cycloalkyl which may be unsubstituted or substituted by C₁₋₃alkyl, or OH; Z is CH or N; and V is NR⁵⁶ or CHR⁵⁷; R⁵⁴ and R⁵⁵ are independently H, C₁₋₃alkyl,
R⁵², R⁵³ R⁵⁶, and R⁵⁷ are independently H, C₁₋₈alkyl, C₃-i₄cycloalkyl, C₁₋₈haloalkyl, NR⁵⁸R⁵⁹, C(O)OR⁶⁰, C(O)C₁₋₈alkyl, Co-₈alkylC(O)C₀-8alkyl-NR⁶¹R⁶², C₁₋₈alkoxy, C₁₋₈alkyl0R⁶³, C(O)-5-Hcycloheteroalkyl group, C_{3-H}Cycloalkyl group, each of which when not H may be substituted by one or more of C₁-8alky, OH, or CN; R⁵⁸, R⁵⁹, R⁶⁰, R⁶¹, R⁶², and R⁶³ are H or C₁₋₈alkyl.

For example, the CDK inhibitor may be selected from: 7-Cyclopentyl-2-[5-(3-methyl-piperazin-1-yl)-pyridin-2-ylamino]-7H-pyrrolo[2,3d]pyrimidine-6-carbonitrile; 7-Cyclopentyl-2-{5-[4-(2-fluoro-ethyl)-piperazin-1-yl]-pyridin-2-ylamino}-7H-pyrrolo[2,3-d]pyrimidine-6-carboxylic acid dimethylamide;7-Cyclopentyl-2-(4-dimethylamino-3,4,5,6-tetrahydro-2H-[1,3′]bipyridinyl-6′-ylamino)-7H-pyrrolo[2,3-d]pyrimidine-6-carboxylic acid dimethylamide;2-[5-(4-Carbamoylmethyl-piperazin-1-yl)-pyridin-2-ylamino]-7-cyclopentyl-7H-pyrrolo[2,3-d]pyrimidine-6-carboxylic acid dimethylamide;2-{5-[4-(2-Amino-acetyl)-piperazin-1-yl]-pyridin-2-ylamino}-7-cyclopentyl-7H-pyrrolo[2,3-d]pyrimidine-6-carboxylic acid dimethylamide;2-[5-(3-Amino-pyrrolidin-1-yl)-pyridin-2-ylamino]-7-cyclopentyl-7H-pyrrolo[2,3-d]pyrimidine-6-carboxylic acid dimethylamide;7-Cyclopentyl-2-{5-[4-(2-methoxy-ethyl)-piperazin-1-yl]-pyridin-2-ylamino}-7H-pyrrolo[2,3-d]pyrimidine-6-carboxylic acid dimethylamide;7-Cyclopentyl-2-[4-(2-hydroxyethyl)-3,4,5,6-tetrahydro-2H-[1,2′]bipyrazinyl-5′-ylamino]-7H-pyrrolo[2,3-d]pyrimidine-6-carboxylic acid dimethylamide; 7-Cyclopentyl-2-[5-((R)-3-methyl-piperazin-1-yl)-pyridin-2-ylamino]-7H-pyrrolo[2,3-d]pyrimidine-6-carboxylic acid dimethylamide;7-Cyclopentyl-2-[5-((S)-3-methylpiperazin-1-yl)-pyridin-2-ylamino]-7H-pyrrolo[2,3-d]pyrimidine-6-carboxylic acid dimethylamide;7-Cyclopentyl-2-[5-(3-methylpiperazin-1-yl)-pyridin-2-ylamino]-7H-pyrrolo[2,3-d]pyrimidine-6-carboxylic acid dimethylamide;7-Cyclopentyl-2-{5-[4-(3-hydroxypropyl)-piperazin-1-yl]-pyridin-2-ylamino}-7H-pyrrolo[2,3-d]pyrimidine-6-carboxylic acid dimethylamide;7-Cyclopentyl-2-{5-[4-(pyrrolidine-1-carbonyl)-piperazin-1-yl]-pyridin-2-ylamino}-7H-pyrrolo[2,3-d]pyrimidine-6-carboxylic acid dimethylamide;7-Cyclopentyl-2-{5-[4-(2-hydroxy-ethyl)-piperazin-1-yl]-pyridin-2-ylamino}-7H-pyrrolo[2,3-d]pyrimidine-6-carboxylic acid dimethylamide;7-Cyclopentyl-2-{5-[4-((S)-2,3-dihydroxypropyl)-piperazin-1-yl]-pyridin-2-ylamino}-7H-pyrrolo[2,3-d]pyrimidine-6-carboxylic acid dimethylamide; 7-Cyclopentyl-2-(5-{4-[2-(2-hydroxyethoxy)-ethyl]-piperazin-1-yl}-pyr idin-2-ylamino)-7H-pyrrolo[2,3-d]pyrimidine-6-carboxylic acid dimethylamide; 7-Cyclopentyl-2-{5-[4-(2-hydroxy-1-methylethyl)-piperazin-1-yl]-pyridin-2-ylamino}-7H-pyrrolo[2,3-d]pyrimidine-6-carboxylic acid dimethylamide;7-Cyclopentyl-2-{6-[4-(2-hydroxyethyl)-piperazin-1-yl]-pyridazin-3-ylamino}-7H-pyrrolo [2,3-d]pyrimidine-6-carboxylic acid dimethylamide;7-Cyclopentyl-2-{5-[4-(2,3-dihydroxypropyl)-piperazin-1-yl]-pyridin-2-ylamino}-7H-pyrrolo[2,3-d]pyrimidine-6-carboxylic acid dimethylamide;7-Cyclopentyl-2-{5-[4-((R)-2,3-dihydroxypropyl)-piperazin-1-yl]-pyridin-2-ylamino}-7H-pyrrolo[2,3-d]pyrimidine-6-carboxylic acid dimethylamide;7-Cyclopentyl-2-(4-dimethylamino-3,4,5,6-tetrahydro-2H-[1,3′]bipyridinyl-6′-ylamino)-7H-pyrrolo[2,3-d]pyrimidine-6-carbonitrile;7-Cyclopentyl-2-(3,4,5,6-tetrahydro-2H-[1,2′]bipyrazinyl-5′-ylamino)-7H-pyrrolo[2,3-d]pyrimidine-6-carboxylic acid dimethylamide;7-Cyclopentyl-2-[5-(piperazine-1-carbonyl)-pyridin-2-ylamino]-7H-pyrrolo[2,3d]pyrimidine-6-carboxylic acid dimethylamide;7-Cyclopentyl-2-[5-(4-dimethylaminopiperidine-1-carbonyl)-pyridin-2-ylamino]-7H-pyrrolo[2,3-d]pyrimidine-6-carboxylic acid dimethylamide;7-Cyclopentyl-2-(1′,2^{>},3^{>},4^{>},5^{>},6′-hexahydro-[3,4^{>}]bipyridinyl-6-ylamino)-7H-pyrrolo[2,3d]pyrimidine-6-carboxylic acid dimethylamide;7-Cyclopentyl-2-[5-((S)-3-methylpiperazin-1-ylmethyl)-pyridin-2-ylamino]-7Hpyrrolo[2,3-d]pyrimidine-6-carboxylic acid dimethylamide;7-Cyclopentyl-2-{5-[4-((S)-2-hydroxypropyl)-piperazin-1-yl]-pyridin-2-ylamino}-7H-pyrrolo[2,3-d]pyrimidine-6-carboxylic acid dimethylamide;7-Cyclopentyl-2-{5-[4-((R)-2-hydroxypropyl)-piperazin-1-yl]-pyridin-2-ylamino}-7H-pyrrolo [2,3-d]pyrimidine-6-carboxylic acid dimethylamide;7-Cyclopentyl-2-(5-piperazin-1-yl-pyridin-2-ylamino)-7H-pyrrolo[2,3-d]pyrimidine-6-carboxylic acid methylamide;7-Cyclopentyl-2-[5-(4-isopropyl-piperazin-1-yl)-pyridin-2-ylamino]-7H-pyrrolo[2,3d]pyrimidine-6-carboxylic acid dimethylamide;7-Cyclopentyl-2-[5-(4-isopropyl-piperazine-1-carbonyl)-pyridin-2-ylamino]-7H-pyrrolo[2,3-d]pyrimidine-6-carboxylic acid dimethylamide; 7-Cyclopentyl-2-{5-[4-(4-methyl-pentyl)-piperazin-1-yl]-pyridin-2-ylamino}-7H-pyrrolo[2,3-d]pyrimidine-6-carboxylic acid dimethylamide;7-Cyclopentyl-2-[6-(4-isopropyl-piperazin-1-yl)-pyridazin-3-ylamino]-7H-pyrrolo[2,3-d]pyrimidine-6-carboxylic acid dimethylamide;7-Cyclopentyl-2-{5-[4-(2-hydroxy-2methylpropyl)-piperazin-1-yl]-pyridin-2-ylamino}-7H-pyrrolo[2,3-d]pyrimidine-6-carboxylic acid dimethylamide;7-Cyclopentyl-2-[5-(3,3-dimethyl-piperazin-1-yl)-pyridin-2-ylamino]-7H-pyrrolo[2,3-d]pyrimidine-6-carboxylic acid dimethylamide;7-Cyclopentyl-2-[5-(3,8-diaza-bicyclo[3.2.1]oct-3-ylmethyl)-pyridin-2-ylamino]-7H-pyrrolo[2,3-d]pyrimidine-6-carboxylic acid dimethylamide;7-Cyclopentyl-2-(5-piperazin-1-yl-pyridin-2-ylamino)-7H-pyrrolo[2,3-d]pyrimidine-6-carboxylic acid dimethylamide;7-Cyclopentyl-2-[5-(4-ethyl-piperazin-1-yl)-pyridin-2-ylamino]-7H-pyrrolo[2,3-d]pyrimidine-6-carboxylic acid dimethylamide;7-Cyclopentyl-2-[5-(4-cyclopentyl-piperazin-1-yl)-pyridin-2-ylamino]-7H-pyrrolo[2,3-d]pyrimidine-6-carboxylic acid dimethylamide;7-Cyclopentyl-2-(1′-isopropyl-1′,2′,3′,4′,5′,6′-hexahydro-[3,4′]bipyridinyl-6-ylamino)-7H-pyrrolo[2,3-d]pyrimidine-6-carboxylic acid dimethylamide;7-Cyclopentyl-2-{5-[(R)-4-(2-hydroxyethyl)-3-methyl-piperazin-1-yl]-pyridin-2-ylamino}-7H-pyrrolo[2,3-d]pyrimidine-6-carboxylic acid dimethylamide;7-Cyclopentyl-2-{5-[(S)-4-(2-hydroxy ethyl)-3-methyl-piperazin-1-yl]-pyridin-2-ylamino}-7H-pyrrolo[2,3-d]pyrimidine-6-carboxylic acid dimethylamide;7-Cyclopentyl-2-{5-[4-(2-hydroxyethyl)-piperazin-1-ylmethyl]-pyridin-2-ylamino}-7H-pyrrolo[2,3-d]pyrimidine-6-carboxylic acid dimethylamide;7-Cyclopentyl-2-{5-[4-(2-dimethylaminoacetyl)-piperazin-1-yl]-pyridin-2-ylamino}-7H-pyrrolo[2,3-d]pyrimidine-6-carboxylic acid dimethylamide;7-Cyclopentyl-2-{5-[4-(2-ethyl-butyl)piperazin-1-yl]-pyridin-2-ylamino}-7H-pyrrolo[2,3-d]pyrimidine-6-carboxylic acid dimethylamide;2-{5-[4-(2-Cyclohexyl-acetyl)piperazin-1-yl]-pyridin-2-ylamino}-7-cyclopentyl-7H-pyrrolo[2,3-d]pyrimidine-6-carboxylic acid dimethylamide; 7-Cyclopentyl-2-{5-[4-(3-cyclopentyl-propionyl)-piperazin-1-yl]-pyridin-2-ylamino}7H-pyrrolo[2,3-d]pyrimidine-6-carboxylic acid dimethylamide;7-Cyclopentyl-2-[5-(4-isobutylpiperazin-1-yl)-pyridin-2-ylamino]-7H-pyrrolo[2,3d]pyrimidine-6-carboxylic acid dimethylamide;{4-[6-(7-Cyclopentyl-6-dimethylcarbamoyl-7H-pyrrolo[2,3-d]pyrimidin-2-ylamino)pyridin-3-yl]-piperazin-1-yl}-acetic acid methyl ester;7-Cyclopentyl-2-{5-[4-(2-isopropoxyethyl)-piperazin-1-yl]-pyridin-2-ylamino}-7Hpyrrolo[2,3-d]pyrimidine-6-carboxylic acid dimethylamide;{4-[6-(7-Cyclopentyl-6-dimethylcarbamoyl-7H-pyrrolo[2,3-d]pyrimidin-2-ylamino)pyridin-3-yl]-piperazin-1-yl}-acetic acid ethyl ester;4-(6-{7-Cyclopentyl-6-[(2-hydroxy-ethyl)methyl-carbamoyl]-7H-pyrrolo[2,3-d]pyrimidin-2-ylamino}-pyridin-3-yl)piperazine-1-carboxylic acid tert-butyl ester;7-Cyclopentyl-2-{5-[4-(2-methyl-butyl)piperazin-1-yl]-pyridin-2-ylamino}-7H-pyrrolo[2,3-d]pyrimidine-6-carboxylic acid dimethylamide;7-Cyclopentyl-2-[1′-(2-hydroxy-ethyl)-1′,2′,3′,4′,5′,6′-hexahydro-[3,4′]bipyridinyl-6-ylamino]-7H-pyrrolo[2,3-d]pyrimidine-6-carboxylic acid dimethylamide;{4-[6-(7-Cyclopentyl-6-dimethylcarbamoyl-7H-pyrrolo[2,3-d]pyrimidin-2-ylamino)-pyridin-3-yl]piperazin-1-yl}-acetic acid; and 2-{4-[6-(7-Cyclopentyl-6-dimethylcarbamoyl-7H-pyrrolo[2,3-d]pyrimidin-2-ylamino)-pyridin-3-yl]-piperazin-1-yl}-propionic acid; or pharmaceutically acceptable salts thereof.

For example, the CDK inhibitor may be Ribociclib.

For example, the CDK inhibitor may be used for modulating the activity of protein kinases, such as CDK1, CDK2, CDK4, CDK5, CDK6, CDK7, CDK8 and CDK9. For example, the compound may be recorded in WO2010020675A1.

For example, the CDK inhibitor may have a structure of formula II.

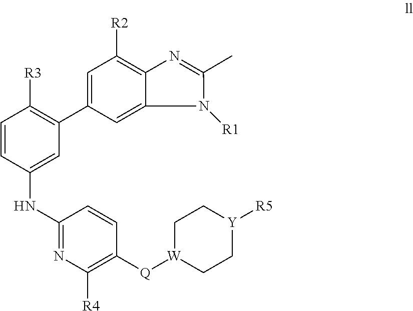

R1 is C₃-C₅ alkyl, C₃-C₅ cycloalkyl or cyclopropyl-methyl; R2 and R3 are H or fluorine, wherein at least one of R2 or R3 is fluorine; R4 is H or CH₃; R5 is C1-C6 alkyl or —NR6R7 wherein R6 and R7 are C1-C3 alkyl; Q is CH₂, O, S or a direct bond; and W and Y are C or N, wherein at least one of W or Y is N and wherein when Q is O or S, W is C; or a pharmaceutically acceptable salt thereof.

For example, R may be is isopropyl, cyclopropyl, cyclopentyl or cyclopropyl-methyl.

For example, R may be is isopropyl.

For example, R2 and R3 may each be fluorine.

For example, R4 may be H.

For example, R5 may be C₁-C₃ alkyl.

For example, Q may be CH₂ or a direct bond.

For example, Q may be CH₂.

For example, Y may be N.

For example, W may be N.

For example, the CDK inhibitor may be selected from the group consisting of:

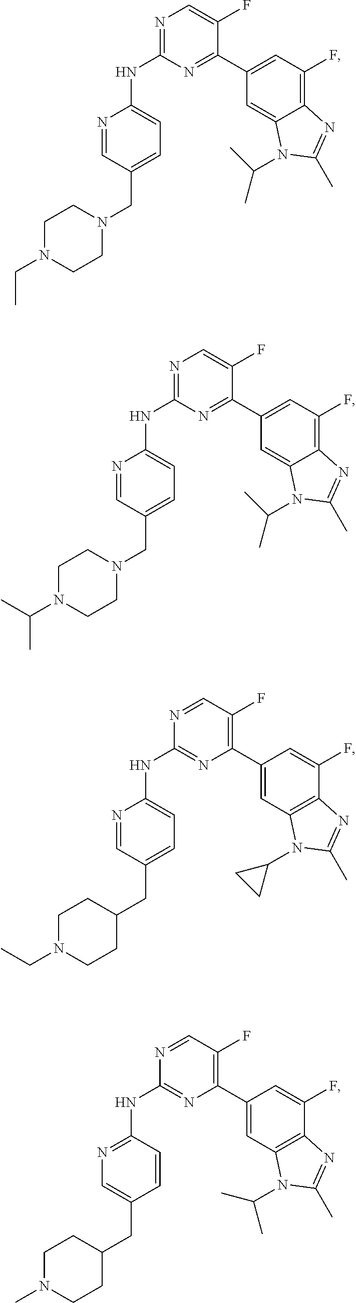 [SMILES: CCN1CCN(Cc2ccc(Nc3ncc(F)c(-c4cc(F)c5nc(C)n(C(C)C)c5c4)n3)nc2)CC1 ; Cc1nc2c(F)cc(-c3nc(Nc4ccc(CN5CCN(C(C)C)CC5)cn4)ncc3F)cc2n1C(C)C ; CCN1CCC(Cc2ccc(Nc3ncc(F)c(-c4cc(F)c5nc(C)n(C6CC6)c5c4)n3)nc2)CC1 ; Cc1nc2c(F)cc(-c3nc(Nc4ccc(CC5CCN(C)CC5)cn4)ncc3F)cc2n1C(C)C]

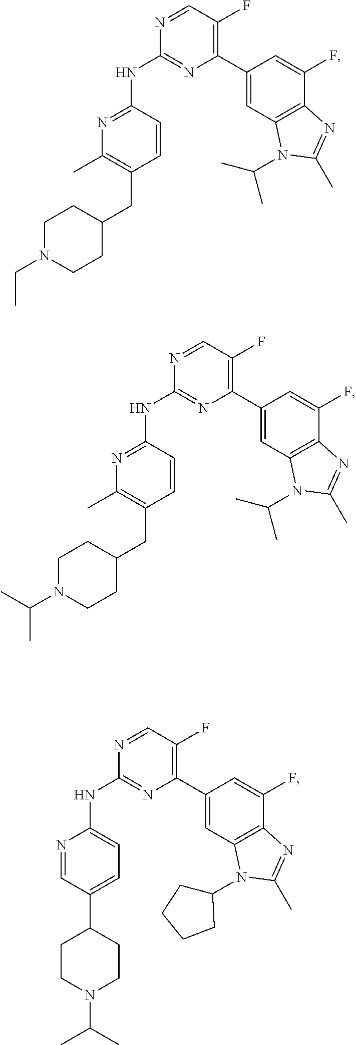 [SMILES: CCN1CCC(Cc2ccc(Nc3ncc(F)c(-c4cc(F)c5nc(C)n(C(C)C)c5c4)n3)nc2C)CC1 ; Cc1nc(Nc2ncc(F)c(-c3cc(F)c4nc(C)n(C(C)C)c4c3)n2)ccc1CC1CCN(C(C)C)CC1 ; Cc1nc2c(F)cc(-c3nc(Nc4ccc(C5CCN(C(C)C)CC5)cn4)ncc3F)cc2n1C1CCCC1]

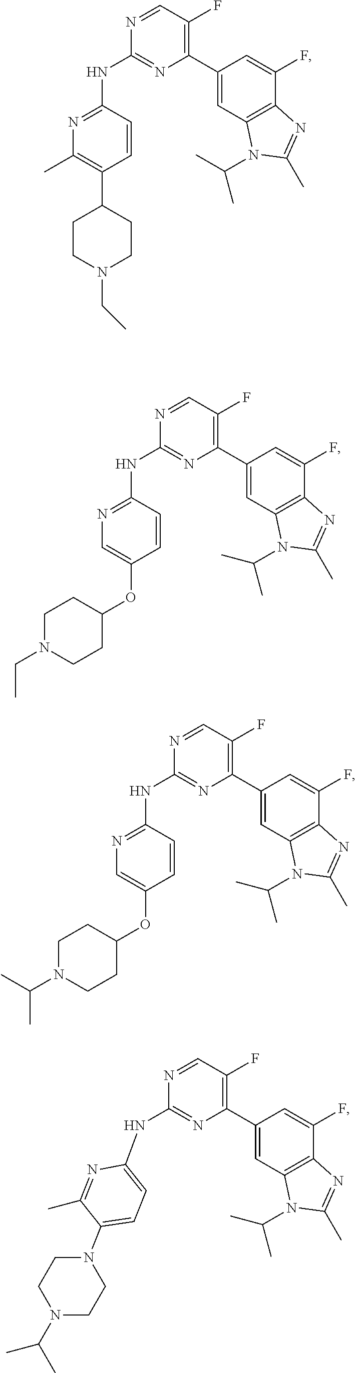 [SMILES: CCN1CCC(c2ccc(Nc3ncc(F)c(-c4cc(F)c5nc(C)n(C(C)C)c5c4)n3)nc2C)CC1 ; CCN1CCC(Oc2ccc(Nc3ncc(F)c(-c4cc(F)c5nc(C)n(C(C)C)c5c4)n3)nc2)CC1 ; Cc1nc2c(F)cc(-c3nc(Nc4ccc(OC5CCN(C(C)C)CC5)cn4)ncc3F)cc2n1C(C)C ; Cc1nc(Nc2ncc(F)c(-c3cc(F)c4nc(C)n(C(C)C)c4c3)n2)ccc1N1CCN(C(C)C)CC1]

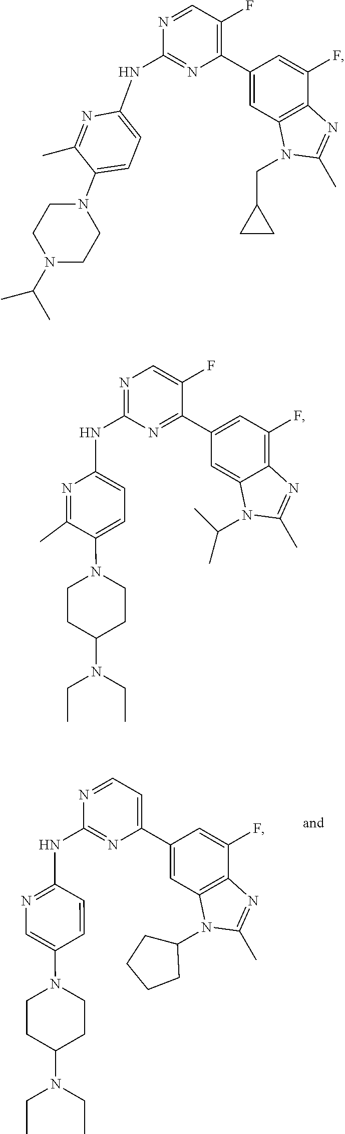 [SMILES: Cc1nc(Nc2ncc(F)c(-c3cc(F)c4nc(C)n(CC5CC5)c4c3)n2)ccc1N1CCN(C(C)C)CC1 ; CCN(CC)C1CCN(c2ccc(Nc3ncc(F)c(-c4cc(F)c5nc(C)n(C(C)C)c5c4)n3)nc2C)CC1 ; CCN(CC)C1CCN(c2ccc(Nc3nccc(-c4cc(F)c5nc(C)n(C6CCCC6)c5c4)n3)nc2)CC1]

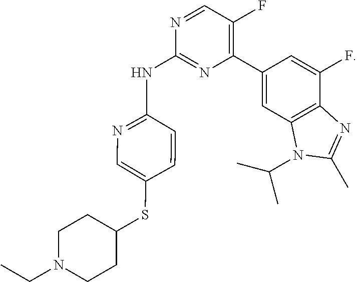 [SMILES: CCN1CCC(Sc2ccc(Nc3ncc(F)c(-c4cc(F)c5nc(C)n(C(C)C)c5c4)n3)nc2)CC1]

For example, the CDK inhibitor may be

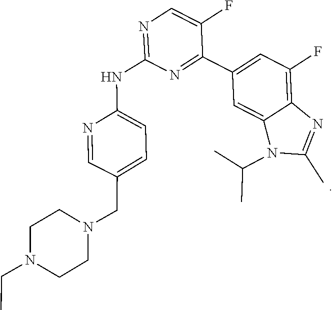 [SMILES: CCN1CCN(Cc2ccc(Nc3ncc(F)c(-c4cc(F)c5nc(C)n(C(C)C)c5c4)n3)nc2)CC1]

For example, the CDK inhibitor may be Abemaciclib.

For example, the CDK inhibitor may be CDK4/6 inhibitors having secondary inhibitory activity at other non-Cdk kinases. For example, the compound may be recorded in WO2010075074A1.

For example, the CDK inhibitor may have a structure of formula III,

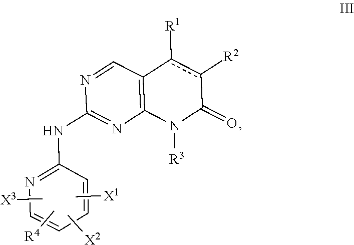

wherein the HER2 inhibitor comprises a first antigen binding domain and a second antigen binding domain, the first antigen binding domain is capable of specifically binding to a first HER2 antigen, the second antigen binding domain is capable of specifically binding to a second HER2 antigen, and the first HER2 antigen and the second HER2 antigen are not identical; X¹, X², and X³ are independently hydrogen, halogen, C₁-C₆ alkyl, C₁-C₆ halo alkyl, C₁-C₈ alkoxy, C₁-C₈ alkoxyalkyl, CN, NO₂, OR⁵, NR⁵R⁶, CO₂R⁵, COR⁵, S(O)ₙR⁵, CONR⁵R⁶, NR⁵COR⁶, NR⁵SO₂R⁶, SO₂NR⁵R⁶, and P(O)(OR⁵)(OR⁶); with the proviso that at least one of X¹, X², and X³ must be hydrogen; n is selected from 0, 1 and 2; R¹ is, in each instance, independently, hydrogen, halogen, C₁-C₆ alkyl, C₁-C₆ halo alkyl, C₁-C₆ hydroxyalkyl, or C₃-C₇ cycloalkyl; R² and R⁴ are each independently selected from hydrogen, halogen, C₁-C₈ alkyl, C₃-C₇ cycloalkyl, C₁-C₈ alkoxy, C₁-C₈ alkoxyalkyl, C₁-C₈ halo alkyl, C₁-C₈ hydroxyalkyl, C₂-C₈ alkenyl, C₂-C₈ alkynyl, nitrile, nitro, OR⁵, SR⁵, NR⁵R⁶, N(O)R⁵R⁶, P(O)(OR⁵)(OR⁶), (CR⁵R⁶)ₘNR⁷R⁸, COR⁵, (CR⁴R⁵)ₘC(O)R⁷, CO₂R⁵, CONR⁵R⁶, C(O)NR⁵SO₂R⁶, NR⁵SO₂R⁶, C(O)NR⁵OR⁶, S(O)ₙR⁵, SO₂NR⁵R⁶, P(O)(OR⁵)(OR⁶), (CR⁵R⁶)ₘP(O)(OR⁷)(OR⁸), (CR⁵R⁶)ₘ-aryl, (CR⁵R⁶)ₘ-heteroaryl, T(CH₂)ₘQR⁵, —C(O)T(CH₂)ₘQR⁵, NR⁵C(O)T(CH₂)ₘQR⁵, and —CR⁵═CR⁶C(O)R⁷; or R¹ and R² may form a carbocyclic group containing 3-7 ring members, preferably 5-6 ring members, up to four of which can optionally be replaced with a heteroatom independently selected from oxygen, sulfur, and nitrogen, and wherein the carbocyclic group is unsubstituted or substituted with one, two, or three groups independently selected from halogen, hydroxy, hydroxyalkyl, nitrile, lower C₁-C₈ alkyl, lower C₁-C₈ alkyl, alkoxycarbonyl, alkylcarbonyl, alkylcarbonylamino, aminoalkyl, trifluoromethyl, N-hydroxyacetamide, trifluoromethylalkyl, amino, and mono or dialkylamino, (CH₂)ₘC(O)NR⁵R⁶, and O(CH₂)ₘC(O)OR⁵, provided, however, that there is at least one carbon atom in the carbocyclic ring and that if there are two or more ring oxygen atoms, the ring oxygen atoms are not adjacent to one another; T is O, S, NR⁷, N(O)R⁷, NR⁷R⁸W, or CR⁷R⁸; Q is O, S, NR⁷, N(O)R⁷, NR⁷R⁸W, CO₂, O(CH₂)ₘ-heteroaryl, O(CH₂)ₘS(O)ₙR⁸, (CH₂)-heteroaryl, or a carbocyclic group containing from 3-7 ring members, up to four of which ring members are optionally heteroatoms independently selected from oxygen, sulfur, and nitrogen, provided, however, that there is at least one carbon atom in the carbocyclic ring and that if there are two or more ring oxygen atoms, the ring oxygen atoms are not adjacent to one another, wherein the carbocyclic group is unsubstituted or substituted with one, two, or three groups independently selected from alkoxycarbonyl, alkylcarbonyl, alkylcarbonylamino, aminoalkyl, trifluoromethyl, N-hydroxyacetamide, trifluoromethylalkyl, amino, and mono or dialkylamino; W is an anion selected from the group consisting of chloride, bromide, trifluoroacetate, and triethylammonium; m is selected from 0, 1, 2, 3, 4, 5 and 6; R⁴ and one of X¹, X² and X³ may form an aromatic ring containing up to three heteroatoms independently selected from oxygen, sulfur, and nitrogen, and optionally substituted by up to 4 groups independently selected from alogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, mono- or dialkylamino, N-hydroxyacetamido, aryl, heteroaryl, carboxyalkyl, nitrile, NR⁷SO₂R⁸, C(O)NR⁷R⁸, NR⁷C(O)R⁸, C(O)OR⁷, C(O)NR⁷SO₂R⁸, (CH₂)ₘS(O)ₙR⁷, (CH₂)ₘ-heteroaryl, O(CH₂)ₘ-heteroaryl, (CH₂), C(O)NR⁷R⁸, O(CH₂)ₘC(O)OR⁷, (CH₂)ₘSO₂NR⁷R⁸, and C(O)R⁷; R³ is hydrogen, aryl, C₁-C₈ alkyl, C₁-C₈ alkyl, C₃-C₇ cycloalkyl, or C₃-C₇-heterocyclyl; R⁵ and R⁶ are each independently hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl; or R⁵ and R⁶, when attached to the same nitrogen atom, taken together with the nitrogen to which they are attached, form a heterocyclic ring containing from 3-8 ring members, up to four of which members can optionally be replaced with heteroatoms independently selected from oxygen, sulfur, S(O), S(O)₂, and nitrogen, provided, however, that there is at least one carbon atom in the heterocyclic ring and that if there are two or more ring oxygen atoms, the ring oxygen atoms are not adjacent to one another, wherein the heterocyclic group is unsubstituted or substituted with one, two or three groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, nitrile, mono- or dialkylamino, N-hydroxyacetamido, aryl, heteroaryl, carboxyalkyl, NR⁷SO₂R⁸, C(O)NR⁷R⁸, NR⁷C(O)R⁸, C(O)OR⁷, C(O)NR⁷SO₂R⁸, (CH₂)ₘS(O)ₙR⁷, (CH₂)ₘ-heteroaryl, O(CH₂)ₘ-heteroaryl, (CH₂)ₘC(O)NR⁷R⁸, O(CH₂)ₘC(O)OR⁷, and (CH₂)SO₂NR⁷R⁸; R⁷ and R⁸ are, each independently, hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl; or R⁷ and R⁸, when attached to the same nitrogen atom, taken together with the nitrogen to which they are attached, may form a heterocyclic ring containing from 3-8 ring members, up to four of which members are optionally heteroatoms independently selected from oxygen, sulfur, S(O), S(O)₂, and nitrogen, provided, however, that there is at least one carbon atom in the heterocyclic ring and that if there are two or more ring oxygen atoms, the ring oxygen atoms are not adjacent to one another, wherein the heterocyclic group is unsubstituted or substituted with one, two or three groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, nitrile, mono- or dialkylamino, N-hydroxyacetamido, aryl, heteroaryl, carboxyalkyl; and the pharmaceutically acceptable salts, esters, amides, and prodrugs thereof.

For example, the compound may have a structure of formula IV:

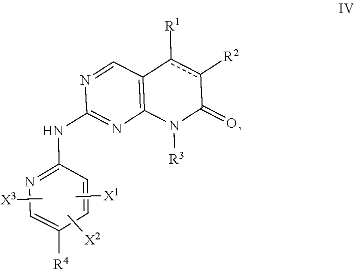

wherein X¹, X², and X³ are independently hydrogen, halogen, C₁-C₆ alkyl, C₁-C₆ halo alkyl, C₁-C₈ alkoxy, C₁-C₈ alkoxyalkyl, CN, NO₂, OR⁵, NR⁵R⁶, CO₂R⁵, COR⁵, S(O)ₙR⁵, CONR⁵R⁶, NR⁵COR⁶, NR⁵SO₂R⁶, SO₂NR⁵R⁶, and P(O)(OR⁵)(OR⁶); with the proviso that at least one of X¹, X², and X³ must be hydrogen; n is selected from 0, 1 and 2; R¹ is, in each instance, independently, hydrogen, halogen, C₁-C₆ alkyl, C₁-C₆ halo alkyl, C₁-C₆ hydroxyalkyl, or C₃-C₇ cycloalkyl; R² and R⁴ are each independently selected from hydrogen, halogen, C₁-C₈ alkyl, C₃-C₇ cycloalkyl, C₁-C₈ alkoxy, C₁-C₈ alkoxyalkyl, C₁-C₈ halo alkyl, C₁-C₈ hydroxyalkyl, C₂-C₈ alkenyl, C₂-C₈ alkynyl, nitrile, nitro, OR⁵, SR⁵, NR⁵R⁶, N(O)R⁵R⁶, P(O)(OR⁵)(OR⁶), (CR⁵R⁶)ₘNR⁷R⁸, COR⁵, (CR⁴R⁵)ₘC(O)R⁷, CO₂R5, CONR⁵R⁶, C(O)NR⁵SO₂R⁶, NR⁵SO₂R⁶, C(O)NR⁵OR⁶, S(O)ₙR⁵, SO₂NR⁵R⁶, P(O)(OR⁵)(OR⁶), (CR⁵R⁶)ₘP(O)(OR⁷)(OR⁸), (CR⁵R⁶)ₘ-aryl, (CR⁵R⁶)ₘ-heteroaryl, T(CH₂)ₘQR⁵, —C(O)T(CH₂)ₘQR⁵, NR⁵C(O)T(CH₂)ₘQR⁵, and —CR⁵═CR⁶C(O)R⁷; or R¹ and R² may form a carbocyclic group containing 3-7 ring members, preferably 5-6 ring members, up to four of which can optionally be replaced with a heteroatom independently selected from oxygen, sulfur, and nitrogen, and wherein the carbocyclic group is unsubstituted or substituted with one, two, or three groups independently selected from halogen, hydroxy, hydroxyalkyl, nitrile, lower C₁-C₈ alkyl, lower C₁-C₈ alkyl, alkoxycarbonyl, alkylcarbonyl, alkylcarbonylamino, aminoalkyl, trifluoromethyl, N-hydroxyacetamide, trifluoromethylalkyl, amino, and mono or dialkylamino, (CH₂)ₘC(O)NR⁵R⁶, and O(CH₂)ₘC(O)OR⁵, provided, however, that there is at least one carbon atom in the carbocyclic ring and that if there are two or more ring oxygen atoms, the ring oxygen atoms are not adjacent to one another; T is O, S, NR⁷, N(O)R⁷, NR⁷R⁸W, or CR⁷R⁸; Q is O, S, NR⁷, N(O)R⁷, NR⁷R⁸W, CO₂, O(CH₂)ₘ-heteroaryl, O(CH₂)ₘS(O)ₙR⁸, (CH₂)-heteroaryl, or a carbocyclic group containing from 3-7 ring members, up to four of which ring members are optionally heteroatoms independently selected from oxygen, sulfur, and nitrogen, provided, however, that there is at least one carbon atom in the carbocyclic ring and that if there are two or more ring oxygen atoms, the ring oxygen atoms are not adjacent to one another, wherein the carbocyclic group is unsubstituted or substituted with one, two, or three groups independently selected from alkoxycarbonyl, alkylcarbonyl, alkylcarbonylamino, aminoalkyl, trifluoromethyl, N-hydroxyacetamide, trifluoromethylalkyl, amino, and mono or dialkylamino; W is an anion selected from the group consisting of chloride, bromide, trifluoroacetate, and triethylammonium; m is selected from 0, 1, 2, 3, 4, 5 and 6; R⁴ and one of X¹, X² and X³ may form an aromatic ring containing up to three heteroatoms independently selected from oxygen, sulfur, and nitrogen, and optionally substituted by up to 4 groups independently selected from alogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, mono- or dialkylamino, N-hydroxyacetamido, aryl, heteroaryl, carboxyalkyl, nitrile, NR⁷SO₂R⁸, C(O)NR⁷R⁸, NR⁷C(O)R⁸, C(O)OR⁷, C(O)NR⁷SO₂R⁸, (CH₂)ₘS(O)ₙR⁷, (CH₂)ₘ-heteroaryl, O(CH₂)ₘ-heteroaryl, (CH₂), C(O)NR⁷R⁸, O(CH₂)ₘC(O)OR⁷, (CH₂)ₘSO₂NR⁷R⁸, and C(O)R⁷; R³ is hydrogen, aryl, C₁-C₈ alkyl, C₁-C₈ alkyl, C₃-C₇ cycloalkyl, or C₃-C₇-heterocyclyl; R⁵ and R⁶ are each independently hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl; or R⁵ and R⁶, when attached to the same nitrogen atom, taken together with the nitrogen to which they are attached, form a heterocyclic ring containing from 3-8 ring members, up to four of which members can optionally be replaced with heteroatoms independently selected from oxygen, sulfur, S(O), S(O)₂, and nitrogen, provided, however, that there is at least one carbon atom in the heterocyclic ring and that if there are two or more ring oxygen atoms, the ring oxygen atoms are not adjacent to one another, wherein the heterocyclic group is unsubstituted or substituted with one, two or three groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, nitrile, mono- or dialkylamino, N-hydroxyacetamido, aryl, heteroaryl, carboxyalkyl, NR⁷SO₂R⁸, C(O)NR⁷R⁸, NR⁷C(O)R⁸, C(O)OR⁷, C(O)NR⁷SO₂R⁸, (CH₂)ₘS(O)ₙR⁷, (CH₂)ₘ-heteroaryl, O(CH₂)ₘ-heteroaryl, (CH₂)ₘC(O)NR⁷R⁸, O(CH₂)ₘC(O)OR⁷, and (CH₂)SO₂NR⁷R⁸; R⁷ and R⁸ are, each independently, hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl; or R⁷ and R⁸, when attached to the same nitrogen atom, taken together with the nitrogen to which they are attached, may form a heterocyclic ring containing from 3-8 ring members, up to four of which members are optionally heteroatoms independently selected from oxygen, sulfur, S(O), S(O)₂, and nitrogen, provided, however, that there is at least one carbon atom in the heterocyclic ring and that if there are two or more ring oxygen atoms, the ring oxygen atoms are not adjacent to one another, wherein the heterocyclic group is unsubstituted or substituted with one, two or three groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, nitrile, mono- or dialkylamino, N-hydroxyacetamido, aryl, heteroaryl, carboxyalkyl; and the pharmaceutically acceptable salts, esters, amides, and prodrugs thereof.

For example, R³ may be a cyclopentyl group.

For example, R¹ may be alkyl.

For example, R¹ may be methyl.

For example, R² may be (CO)CH₃.

For example, the compound may be selected from a group consisting of: 8-Cyclopentyl-2-(pyridin-2-ylamino)-8H-pyrido[2,3-d]pyrimidin-7-one, 6-Bromo-8-cyclopentyl-2-(5-piperazin-1-yl-pyridin-2-ylamino)-8H-pyrido [2,3-d]pyrimidin-7-one hydrochloride, 8-Cyclopentyl-6-ethyl-2-(5-piperazin-1-yl-pyridin-2-ylamino)-8H-pyrido[2,3-d]pyrimidin-7-one hydrochloride, 8-Cyclopentyl-7-oxo-2-(5-piperazin-1-yl-pyridin-2-ylamino)-7,8-dihydro-pyrido[2,3-d]pyrimidine-6-carboxylic acid ethyl ester hydrochloride, 6-Amino-8-cyclopentyl-2-(5-piperazin-1-yl-pyridin-2-ylamino)-8H-pyrido[2,3-d]pyrimidin-7-one hydrochloride, 6-Bromo-8-cyclopentyl-2-[5-((R)-1-methy-1-pyrrolidin-2-yl)-pyridin-2-ylamino]-8H-pyrido[2,3-d]pyrimidin-7-one hydrochloride, 6-Bromo-8-cyclohexyl-2-(pyridin-2-yl-amino)-8H-pyrido[2,3-d]pyrimidin-7-one, 6-Acetyl-8-cyclopentyl-2-[5-(3,5-dimethyl-piperazin-1-yl)-pyridin-2-ylamino]-5-methyl-8H-pyrido[2,3-d]pyrimidin-7-one, 6-Acetyl-8-cyclopentyl-2-[5-(3,3-dimethyl-piperazin-1-yl)-pyridin-2-ylamino]-5-methyl-8H-pyrido[2,3-d]pyrimidin-7-one, 6-Acetyl-8-cyclopentyl-5-methyl-2-[5-(4-methyl-piperazin-1-yl)-pyridin-2-ylamino]-8H-pyrido[2,3-d]pyrimidin-7-one, 6-Acetyl-2-[5-(3-amino-pyrrolidin-1-yl)-pyridin-2-ylamino]-8-cyclopentyl-5-methyl-8H-pyrido[2,3-d]pyrimidin-7-one, 6-Bromo-8-cyclopentyl-5-methyl-2-(5-morpholin-4-yl-pyridin-2-ylamino)-8H-pyrido[2,3-d]pyrimidin-7-one, 2-{5-[Bis-(2-methoxy-ethyl)-amino]-pyridin-2-ylamino}-6-bromo-8-cyclopentyl-5-methyl-8H-pyrido[2,3-d]pyrimidin-7-one, 6-Acetyl-8-cyclopentyl-5-methyl-2-(5-morpholin-4-yl-pyridin-2-ylamino)-8H-pyrido[2,3-d]pyrimidin-7-one, 6-Acetyl-2-{5-[bis-(2-methoxy-ethyl)-amino]-pyridin-2-ylamino}-8-cyclopentyl-5-methyl-8H-pyrido[2,3-d]pyrimidin-7-one, 4-[6-(8-Cyclopentyl-6-iodo-5-methyl-7-oxo-7,8-dihydro-pyrido[2,3-d]pyrimidin-2-ylamino)-pyridin-3-yl]-piperazine-1-carboxylic acid tert-butyl ester, 8-Cyclopentyl-6-iodo-5-methyl-2-(5-piperazin-1-yl-pyridin-2-ylamino)-8H-pyrido[2,3-d]pyrimidin-7-one, 4-{6-[8-Cyclopentyl-6-(2-ethoxy-ethoxy)-7-oxo-7,8-dihydro-pyrido[2,3-d]pyrimidin-2-ylamino]-pyridin-3-yl}-piperazine-1-carboxylic acid tert-butyl ester, 8-Cyclopentyl-6-(2-ethoxy-ethoxy)-2-(5-piperazin-1-yl-pyridin-2-ylamino)-8H-pyrido[2,3-d]pyrimidin-7-one, 2-{5-[Bis-(2-methoxy-ethyl)-amino]-pyridin-2-ylamino}-6-bromo-8-cyclopentyl-5-methyl-8H-pyrido[2,3-d]pyrimidin-7-one, 6-Acetyl-2-{5-[bis-(2-methoxy-ethyl)-amino]-pyridin-2-ylamino}-8-cyclopentyl-5-methyl-8H-pyrido[2,3-d]pyrimidin-7-one, 4-[6-(8 isopropyl-7-oxo-7,8-dihydro-pyrido[2,3]pyrimidin-2-ylamino)-pyridin-3-yl]-piperazine-1-carboxylic acid tert-butyl ester, 8-isopropyl-2-(5-piperazin-1-yl-pyridin-2-ylamino)-8H-pyrido[2,3-d]pyrimidin-7-one, 4-[6-(8-cyclopentyl-7-oxox-7,8-dihydro-pyrido[2,3-d]pyrimidin-2-ylamino)-pyridin-3-yl]-piperazine-1-carboxylic acid tert-butyl ester, 8-cyclopentyl-2-(5-piperazin-1-yl-pyridin-2-ylamino)-8H-pyrido[2,3-d]pyrimidin-7-one, 4-[6-(8-cyclohexyl-7-oxo-7,8-dihydro-pyrido[2,3-d]pyrimidin-2-ylamino)-pyridin-3-yl]-piperazine-1-carboxylic acid tert-butyl ester, 8-cyclohexyl-2-(5-piperazin-1-yl-pyridin-2-ylamino)-8H-pyrido[2,3-d]pyrimidin-7-one, 4-[6-(8-cyclopropyl-7-oxo-7,8-dihydro-pyrido[2,3-d]pyrimidin-2-ylamino)-pyridin-3-yl]-piperazine-1-carboxylic acid tert-butyl ester, 8-cyclopropyl-2-(5-piperazin-1-yl-pyridin-2-ylamino)-8H-pyrido[2,3-d]pyrimidin-7-one, 6-Bromo-8-cyclopentyl-2-(pyridin-2,6-yldiamino)-8H-pyrido[2,3-d]pyrimidin-7-one, 6-Bromo-8-cyclopentyl-5-methyl-2-(pyridin-2-ylamino)-8H-pyrido[2,3-d]pyrimidin-7-one, 6-Bromo-8-cyclopentyl-5-methyl-2-[5-(4-methyl-piperazin-1-yl)-pyridin-2-ylamino]-8H-pyrido[2,3-d]pyrimidin-7-one, 8-Cyclopentyl-6-(1-ethoxy-vinyl)-5-methyl-2-[5-(4-methyl-piperazin-1-yl)-pyridin-2-ylamino]-8H-pyrido[2,3-d]pyrimidin-7-one, (1-{6-[8-Cyclopentyl-6-(1-ethoxy-vinyl)-5-methyl-7-oxo-7,8-dihydro-pyrido[2,3-d]pyrimidin-2-ylamino]-pyridin-3-yl}-pyrrolidin-3-yl)-carbamic acid tert-butyl ester, 6-Acetyl-8-cyclopentyl-2-(4-hydroxy-3,4,5,6-tetrahydro-2H-[1,3′]bipyridinyl-6′-ylamino)-5-methyl-8H-pyrido[2,3-d]pyrimidin-7-one, 4-[6-(6-Bromo-8-cyclopentyl-5-methyl-7-oxo-7,8-dihydro-pyrido[2,3-d]pyrimidin-2-ylamino)-pyridin-3-yl]-azepane-1-carboxylic acid tert-butyl ester, 6-Bromo-8-cyclopentyl-2-(5-[1,4]diazepan-1-yl-pyridin-2-ylamino)-5-methyl-8H-pyrido[2,3-d]pyrimidin-7-one, 4-{6-[8-Cyclopentyl-6-(1-ethoxy-vinyl)-5-methyl-7-oxo-7,8-dihydro-pyrido[2,3-d]pyrimidin-2-ylamino]-pyridin-3-yl}-[1,4]diazepane-1-carboxylic acid tert-butyl ester, 6-Acetyl-8-cyclopentyl-2-(5-[1,4]diazepan-1-yl-pyridin-2-ylamino)-5-methyl-8H-pyrido[2,3-d]pyrimidin-7-one, 6-Acetyl-8-cyclopentyl-5-methyl-2-(pyridin-2-ylamino)-8H-pyrido[2,3-d]pyriminin-7-one, 4-[6-(8-Cyclopentyl-5-methyl-7-oxo-7,8-dihydro-pyrido[2,3-d]pyrimidin-2-ylamino)-pyridin-3-yl]-piperazine-1-carboxylic acid tert-butyl ester, 8-Cyclopentyl-5-methyl-2-(5-piperazin-4-yl-pyridin-2-ylamino)-8H-pyrido[2,3-d]pyrimidin-7-one, 4-[6-(6-Bromo-8-cyclopentyl-5-methyl-7-oxo-7,8-dihydro-pyrido[2,3-d]pyrimidin-2-ylamino)-pyridin-3-yl]-2,2-dimethyl-piperazine-1-carboxylic acid tert-butyl ester, 6-Bromo-8-cyclopentyl-2-[5-(3,3-dimethyl-piperazin-1-yl)-pyridin-2-ylamino]-5-methyl-8H-pyrido[2,3d]pyrimidin-7-one, 4-{6-[8-Cyclopentyl-6-(1-ethoxy-vinyl)-5-methyl-7-oxo-7,8-dihydro-pyrido[2,3-d]pyrimidin-2-ylamino]-pyridin-3-yl}-2,2-dimethyl-piperazine-1-carboxylic acid tert-butyl ester, 4-[6-(6-Bromo-8-cyclopentyl-5-methyl-7-oxo-7,8-dihydro-pyrido[2,3-d]pyrimidin-2-ylamino)-pyridin-3-yl]-2,6-dimethyl-piperazine-1-carboxylic acid tert-butyl ester, 6-Bromo-8-cyclopentyl-2-[5-(3,5-dimethyl-piperazin-1-yl)-pyridin-2-ylamino]-5-methyl-8H-pyrido[2,3-d]pyrimidin-7-one, 4-{6-[8-Cyclopentyl-6-(1-ethoxy-vinyl)-5-methyl-7-oxo-7,8-dihydro-pyrido[2,3-d]pyrimidin-2-ylamino]-pyridin-3-yl}-2,6-dimethyl-piperazine-1-carboxylic acid tert-butyl ester, 8-Cyclopentyl-6-(1-ethoxy-vinyl)-5-methyl-2-(5-morpholin-4-yl-pyridin-2-ylamino)-8H-pyrido[2,3-d]pyrimidin-7-one, 6-Bromo-8-cyclopentyl-5-methyl-2-(3,4,5,6-tetrahydro-2H-[1,3′]bipyridinyl-6′-ylamino)-8H-pyrido[2,3-d]pyrimidin-7-one, 8-Cyclopentyl-6-(1-ethoxy-vinyl)-5-methyl-2-(3,4,5,6-tetrahydro-2H-[1,3′]bipyridinyl-6′-ylamino)-8H-pyrido[2,3-d]pyrimidin-7-one, 6-Acetyl-8-cyclopentyl-5-methyl-2-(3,4,5,6-tetrahydro-2H-[1,3′]bipyridinyl-6′-ylamino)-8H-pyrido[2,3-d]pyrimidin-7-one, 4-{6-[8-Cyclopentyl-6-(2-ethoxy-ethyl)-7-oxo-7,8-dihydro-pyrido[2,3-d]pyrimidin-2-ylamino]-pyridin-3-yl}-piperazine-1-carboxylic acid tert-butyl ester, 8-Cyclopentyl-6-(2-ethoxy-ethyl)-2-(5-piperazin-1-yl-pyridin-2-ylamino)-8H-pyrido[2,3-d]pyrimidin-7-one, 4-{6-[8-Cyclopentyl-6-(2-methoxy-ethoxymethyl)-7-oxo-7,8-dihydro-pyrido[2,3-d]pyrimidin-2-ylamino]-pyridin-3-yl}-piperazine-1-carboxylic acid tert-butyl ester, 8-Cyclopentyl-6-(2-methoxy-ethoxymethyl)-2-(5-piperazin-1-yl-pyridin-2-ylamino)-8H-pyrido[2,3-d]pyrimidin-7-one, 4-[6-(8-Cyclopentyl-6-ethoxymethyl-7-oxo-7,8-dihydro-pyrido[2,3-d]pyrimidin-2-ylamino)-pyridin-3-yl]-piperazine-1-carboxylic acid tert-butyl ester, 8-Cyclopentyl-6-ethoxymethyl-2-(5-piperazin-1-yl-pyridin-2-ylamino)-8H-pyrido[2,3-d]pyrimidin-7-one, 4-[6-(8-Cyclopentyl-6-methoxymethyl-7-oxo-7,8-dihydro-pyrido[2,3-d]pyrimidin-2-ylamino)-pyridin-3-yl]-piperazine-1-carboxylic acid tert-butyl ester, 8-Cyclopentyl-6-methoxymethyl-2-(5-piperazin-1-yl-pyridin-2-ylamino)-8H-pyrido[2,3-d]pyrimidin-7-one, 6-Bromo-8-cyclopentyl-2-[5-(2,6-dimethyl-morpholin-4-yl)-pyridin-2-ylamino]-5-methyl-8H-pyrido[2,3-d]pyrimidin-7-one, 8-Cyclopentyl-6-ethoxymethyl-2-(3,4,5,6-tetrahydro-2H-[1,3′]bipyridinyl-6′-ylamino)-8H-pyrido[2,3-d]pyrimidin-7-one, 8-Cyclopentyl-6-ethoxymethyl-2-(5-morpholin-4-yl-pyridin-2-ylamino)-8H-pyrido[2,3-d]pyrimidin-7-one, [8-Cyclopentyl-7-oxo-2-(3,4,5,6-tetrahydro-2H-[1,3′]bipyridinyl-6′-ylamino)-7,8-dihydro-pyrido[2,3-d]pyrimidin-6-ylmethyl]-carbamic acid benzyl ester, 8-Cyclopentyl-2-[5-(2,6-dimethyl-morpholin-4-yl)-pyridin-2-ylamino]-6-(1-ethoxy-vinyl)-5-methyl-8H-pyrido[2,3-d]pyrimidin-7-one, 6-Acetyl-8-cyclopentyl-2-[5-(2,6-dimethyl-morpholin-4-yl)-pyridin-2-ylamino]-5-methyl-8H-pyrido[2,3-d]pyrimidin-7-one, 8-Cyclopentyl-5-methyl-2-(5-piperazin-1-yl-pyridin-2-ylamino)-6-propionyl-8H-pyrido[2,3-d]pyrimidin-7-one, 6-Bromo-8-cyclopentyl-2-methyl-8H-pyrido[2,3-d]pyrimidin-7-one, 6-Bromo-8-cyclopentyl-5-methyl-2-(5-piperazin-1-yl-pyridin-2-ylamino)-8H-pyrido[2,3-d]pyrimidin-7-one, 8-Cyclopentyl-6-fluoro-2-(5-piperazin-1-yl-pyridin-2-ylamino)-8H-pyrido[2,3-d]pyrimidin-7-one hydrochloride, 8-Cyclopentyl-6-methyl-2-(5-piperazin-1-yl-pyridin-2-ylamino)-8H-pyrido[2,3-d]pyrimidin-7-one hydrochloride, 8-Cyclopentyl-6-isobutoxy-2-(5-piperazin-1-yl-pyridin-2-ylamino)-8H-pyrido[2,3-d]pyrimidin-7-one hydrochloride, 6-Benzyl-8-cyclopentyl-2-(5-piperazin-1-yl-pyridin-2-ylamino)8H-pyrido[2,3-d]pyrimidin-7-one hydrochloride, 8-Cyclopentyl-6-hydroxymethyl-2-(5-piperazin-1-yl-pyridin-2-ylamino)-8H-pyrido[2,3-d]pyrimidin-7-one hydrochloride, 2-[5-(4-tert-Butoxycarbonyl-piperazin-1-yl)-pyridin-2-ylamino]-8-cyclopentyl-5-methyl-7-oxo-7,8-dihydro-pyrido[2,3-d]pyrimidine-6-carboxylic acid ethyl ester, 6-Acetyl-8-cyclopentyl-2-(5-piperazin-1-yl-pyridin-2-ylamino)-8H-pyrido[2,3-d]pyrimidin-7-one, 6-Acetyl-8-cyclopentyl-5-methyl-2-(5-piperazin-1-yl-pyridin-2-ylamino)-8H-pyrido[2,3-d]pyrimidin-7-one, 6-Bromo-8-cyclopentyl-5-methyl-2-(pyridin-2-ylamino)-8H-pyrido[2,3-d]pyrimidin-7-one, 6-Bromo-8-cyclopentyl-2-(pyridin-2-ylamino)-8H-pyrido[2,3-d]pyrimidin-7-one, 6-Bromo-8-cyclopentyl-2-[5-(3,5-dimethyl-piperazin-1-yl)-pyridin-2-ylamino]-8H-pyrido[2,3-d]pyrimidin-7-one, 6-Bromo-8-cyclopentyl-2-[5-(3,3-dimethyl-piperazin-1-yl)-pyridin-2-ylamino]-8H-pyrido[2,3-d]pyrimidin-7-one, 6-Bromo-8-cyclopentyl-2-[5-(4-methyl-piperazin-1-yl)-pyridin-2-ylamino]-8H-pyrido[2,3-d]pyrimidin-7-one, 2-[5-(3-Amino-pyrrolidin-1-yl)-pyridin-2-ylamino]-6-bromo-8-cyclopentyl-8H-pyrido[2,3-d]pyrimidin-7-one, 6-Bromo-8-cyclopentyl-2-[5-(3-ethylamino-pyrrolidin-1-yl)-pyridin-2-ylamino]-8H-pyrido[2,3-d]pyrimidin-7-one, 6-Bromo-8-cyclopentyl-2-(5-pyrrolidin-1-yl-pyridin-2-ylamino)-8H-pyrido[2,3-d]pyrimidin-7-one, 2-{5-[3-(1-Amino-1-methyl-ethyl)-pyrrolidin-1-yl]-pyridin-2-ylamino}-6-bromo-8-cyclopentyl-8H-pyrido[2,3-d]pyrimidin-7-one, 1-[6-(6-Bromo-8-cyclopentyl-7-oxo-7,8-dihydro-pyrido[2,3-d]pyrimidin-2-ylamino)-pyridin-3-yl]-pyrrolidine-2-carboxylic acid, 6-Bromo-8-cyclopentyl-2-[5-(4-diethylamino-butylamino)-pyridin-2-ylamino]-8H-pyrido[2,3-d]pyrimidin-7-one, 6-Acetyl-8-cyclopentyl-2-[5-(3-ethylamino-pyrimidin-1-yl)-pyridin-2-ylamino]-5-methyl-8H-pyrido[2,3-d]pyrimidin-7-one, 6-Acetyl-8-cyclopentyl-5-methyl-2-(5-pyrrolidin-1-yl-pyridin-2-ylamino)-8H-pyrido[2,3-d]pyrimidin-7-one, 6-Acetyl-2-{5-[3-(1-amino-1-methyl-ethyl)-pyrrolidin-1-yl]-pyridin-2-ylamino}-8-cyclopentyl-5-methyl-8H-pyrido[2,3-d]pyrimidin-7-one, 1-[6-(6-Acetyl-8-cyclopentyl-5-methyl-7-oxo-7,8-dihydro-pyrido[2,3-d]pyrimidin-2-ylamino)-pyridin-3-yl]-pyrrolidine-2-carboxylic acid, 6-Acetyl-8-cyclopentyl-2-[5-(4-diethylamino-butylamino)-pyridin-2-ylamino]-5-methyl-8H-pyrido[2,3-d]pyrimidin-7-one, 8-Cyclopentyl-2-[5-(3,5-dimethyl-piperazin-1-yl)-pyridin-2-ylamino]-6-ethyl-8H-pyrido[2,3-d]pyrimidin-7-one, 8-Cyclopentyl-2-[5-(3,3-dimethyl-piperazin-1-yl)-pyridin-2-ylamino]-6-ethyl-8H-pyrido[2,3-d]pyrimidin-7-one, 8-Cyclopentyl-6-ethyl-2-[5-(4-methyl-piperazin-1-yl)-pyridin-2-ylamino]-8H-pyrido[2,3-d]pyrimidin-7-one, 2-[5-(3-Amino-pyrrolidin-1-yl)-pyridin-2-ylamino]-8-cyclopentyl-6-ethyl-8H-pyrido[2,3-d]pyrimidin-7-one, 8-Cyclopentyl-6-ethyl-2-[5-(3-ethylamino-pyrrolidin-1-yl)-pyridin-2-ylamino]-8H-pyrido[2,3-d]pyrimidin-7-one, 8-Cyclopentyl-6-ethyl-2-(5-pyrrolidin-1-yl-pyridin-2-ylamino)-8H-pyrido[2,3-d]pyrimidin-7-one, 2-{5-[3-(1-Amino-1-methyl-ethyl)-pyrrolidin-1-yl]-pyridin-2-ylamino}-8-cyclopentyl-6-ethyl-8H-pyrido[2,3-d]pyrimidin-7-one, 1-[6-(8-Cyclopentyl-6-ethyl-7-oxo-7,8-dihydro-pyrido[2,3-d]pyrimidin-2-ylamino)-pyridin-3-yl]-pyrrolidine-2-carboxylic acid, 8-Cyclopentyl-2-[5-(4-diethylamino-butylamino)-pyridin-2-ylamino]-6-ethyl-8H-pyrido[2,3-d]pyrimidin-7-one, 6-Benzyl-8-cyclopentyl-2-[5-(3,5-dimethyl-piperazin-1-yl)-pyridin-2-ylamino]-8H-pyrido[2,3-d]pyrimidin-7-one, 6-Benzyl-8-cyclopentyl-2-[5-(3,3-dimethyl-piperazin-1-yl)-pyridin-2-ylamino]-8H-pyrido[2,3-d]pyrimidin-7-one, 6-Benzyl-8-cyclopentyl-2-[5-(4-methyl-piperazin-1-yl)-pyridin-2-ylamino]-8H-pyrido[2,3d]pyrimidin-7-one, 2-[5-(3-Amino-pyrrolidin-1-yl)-pyridin-2-ylamino]-6-benzyl-8-cyclopentyl-8H-pyrido[2,3-d]pyrimidin-7-one, 6-Benzyl-8-cyclopentyl-2-[5-(3-ethylamino-pyrrolidin-1-yl)-pyridin-2-ylamino]-8H-pyrido[2,3-d]pyrimidin-7-one, 6-Benzyl-8-cyclopentyl-2-(5-pyrrolidin-1-yl-pyridin-2-ylamino)-8H-pyrido[2,3-d]pyrimidin-7-one, 2-{5-[3-(1-Amino-1-methyl-ethyl)-pyrrolidin-1-yl]-pyridin-2-ylamino}-6-benzyl-8-cyclopentyl-8H-pyrido[2,3-d]pyrimidin-7-one, 1-[6-(6-Benzyl-8-cyclopentyl-7-oxo-7,8-dihydro-pyrido[2,3-d]pyrimidin-2-ylamino)-pyridin-3-yl]-pyrrolidine-2-carboxylic acid, 6-Benzyl-8-cyclopentyl-2-[5-(4-diethylamino-butylamino)-pyridin-2-ylamino]-8H-pyrido[2,3-d]pyrimidin-7-one, 8-Cyclopentyl-2-[5-(3,5-dimethyl-piperazin-1-yl)-pyridin-2-ylamino]-6-hydroxymethyl-8H-pyrido[2,3-d]pyrimidin-7-one, 8-Cyclopentyl-2-[5-(3,3-dimethyl-piperazin-1-yl)-pyridin-2-ylamino]-6-hydroxymethyl-8H-pyrido[2,3-d]pyrimidin-7-one, 8-Cyclopentyl-6-hydroxymethyl-2-[5-(4-methyl-piperazin-1-yl)-pyridin-2-ylamino]-8H-pyrido[2,3-d]pyrimidin-7-one, 2-[5-(3-Amino-pyrrolidin-1-yl)-pyridin-2-ylamino]-8-cyclopentyl-6-hydroxymethyl-8H-pyrido[2,3-d]pyrimidin-7-one, 8-Cyclopentyl-2-[5-(3-ethylamino-pyrrolidin-1-yl)-pyridin-2-ylamino]-6-hydroxymethyl-8H-pyrido[2,3-d]pyrimidin-7-one, 8-Cyclopentyl-6-hydroxymethyl-2-(5-pyrrolidin-1-yl-pyridin-2-ylamino)-8H-pyrido[2,3-d]pyrimidin-7-one, 2-{5-[3-(1-Amino-1-methyl-ethyl)-pyrrolidin-1-yl]-pyridin-2-ylamino}-8-cyclopentyl-6-hydroxymethyl-8H-pyrido[2,3-d]pyrimidin-7-one, 1-[6-(8-Cyclopentyl-6-hydroxymethyl-7-oxo-7,8-dihydro-pyrido[2,3-d]pyrimidin-2-ylamino)-pyridin-3-yl]-pyrrolidine-2-carboxylic acid, 8-Cyclopentyl-2-[5-(4-diethylamino-butylamino)-pyridin-2-ylamino]-6-hydroxymethyl-8H-pyrido[2,3-d]pyrimidin-7-one, 6-Amino-8-cyclopentyl-2-[5-(3,5-dimethyl-piperazin-1-yl)-pyridin-2-ylamino]-8H-pyrido[2,3-d]pyrimidin-7-one, 6-Amino-8-cyclopentyl-2-[5-(3,3-dimethyl-piperazin-1-yl)-pyridin-2-ylamino]-8H-pyrido[2,3-d]pyrimidin-7-one, 6-Amino-8-cyclopentyl-2-[5-(4-methyl-piperazin-1-yl)-pyridin-2-ylamino]-8H-pyrido[2,3d]pyrimidin-7-one, 6-Amino-2-[5-(3-amino-pyrrolidin-1-yl)-pyridin-2-ylamino]-8-cyclopentyl-8H-pyrido[2,3-d]pyrimidin-7-one, 6-Amino-8-cyclopentyl-2-[5-(3-ethylamino-pyrrolidin-1-yl)-pyridin-2-ylamino]-8H-pyrido[2,3-d]pyrimidin-7-one, 6-Amino-8-cyclopentyl-2-(5-pyrrolidin-1-yl-pyridin-2-ylamino)-81H-pyrido[2,3-d]pyrimidin-7-one, 6-Amino-2-{5-[3-(1-amino-1-methyl-ethyl)-pyrrolidin-1-yl]-pyridin-2-ylamino}-8-cyclopentyl-8H-pyrido[2,3-d]pyrimidin-7-one, 1-[6-(6-Amino-8-cyclopentyl-7-oxo-7,8-dihydro-pyrido[2,3-d]pyrimidin-2-ylamino)-pyridin-3-yl]-pyrrolidine-2-carboxylic acid, 6-Amino-8-cyclopentyl-2-[5-(4-diethylamino-butylamino)-pyridin-2-ylamino]-8H-pyrido[2,3-d]pyrimidin-7-one, 6-Bromo-8-cyclopentyl-2-(3,4,5,6-tetrahydro-2H-[1,3′]bipyridinyl-6′-ylamino)-8H-pyrido[2,3-d]pyrimidin-7-one, 6-Bromo-8-cyclopentyl-2-(5-morpholin-4-yl-pyridin-2-ylamino)-8H-pyrido[2,3-d]pyrimidin-7-one, 6-Bromo-8-cyclopentyl-2-(5-diethylamino-pyridin-2-ylamino)-8H-pyrido[2,3-d]pyrimidin-7-one, 2-{5-[Bis-(2-hydroxy-ethyl)-amino]-pyridin-2-ylamino}-6-bromo-8-cyclopentyl-8H-pyrido[2,3-d]pyrimidin-7-one, 2-{5-[Bis-(2-methoxy-ethyl)-amino]-pyridin-2-ylamino}-6-bromo-8-cyclopentyl-8H-pyrido[2,3-d]pyrimidin-7-one, 2-[5-(2-Amino-ethylamino)-pyridin-2-ylamino]-6-bromo-8-cyclopentyl-8H-pyrido[2,3-d]pyrimidin-7-one, 6-Bromo-8-cyclopentyl-2-(5-dimethylamino-pyridin-2-ylamino)-8H-pyrido[2,3-d]pyrimidin-7-one, N-[6-(6-Bromo-8-cyclopentyl-7-oxo-7,8-dihydro-pyrido[2,3-d]pyrimidin-2-ylamino)-pyridin-3-yl]-N-methyl-acetamide, 6-Bromo-8-cyclopentyl-2-[5-(2-methoxy-ethoxy)-pyridin-2-ylamino]-8H-pyrido[2,3-d]pyrimidin-7-one, 6-Bromo-8-cyclopentyl-2-[5-(2-methoxy-ethoxymethyl)-pyridin-2-ylamino]-8H-pyrido[2,3-d]pyrimidin-7-one, 6-Bromo-8-cyclopentyl-2-[5-(2-diethylamino-ethoxy)-pyridin-2-ylamino]-8H-pyrido[2,3-d]pyrimidin-7-one, 6-Bromo-8-cyclopentyl-2-(5-pyrrolidin-1-yl-pyridin-2-ylamino)-8H-pyrido[2,3-d]pyrimidin-7-one, 6-Bromo-8-cyclopentyl-2-(6-methyl-5-piperazin-1-yl-pyridin-2-ylamino)-8H-pyrido[2,3-d]pyrimidin-7-one, 6-Bromo-8-cyclopentyl-5-methyl-2-(3,4,5,6-tetrahydro-2H-[1,3′]bipyridinyl-6′-ylamino)-8H-pyrido[2,3-d]pyrimidin-7-one, 6-Bromo-8-cyclopentyl-2-(5-diethylamino-pyridin-2-ylamino)-5-methyl-8H-pyrido[2,3-d]pyrimidin-7-one, 2-{5-[Bis-(2-hydroxy-ethyl)-amino]-pyridin-2-ylamino}-6-bromo-8-cyclopentyl-5-methyl-8H-pyrido[2,3-d]pyrimidin-7-one, 2-[5-(2-Amino-ethylamino)-pyridin-2-ylamino]-6-bromo-8-cyclopentyl-5-methyl-8H-pyrido[2,3-d]pyrimidin-7-one, 6-Bromo-8-cyclopentyl-2-(5-dimethylamino-pyridin-2-ylamino)-5-methyl-8H-pyrido[2,3-d]pyrimidin-7-one, N-[6-(6-Bromo-8-cyclopentyl-5-methyl-7-oxo-7,8-dihydro-pyrido[2,3-d]pyrimidin-2-ylamino)-pyridin-3-yl]-N-methyl-acetamide, 6-Bromo-8-cyclopentyl-2-[5-(2-methoxy-ethoxy)-pyridin-2-ylamino]-5-methyl-8H-pyrido[2,3-d]pyrimidin-7-one, 6-Bromo-8-cyclopentyl-2-[5-(2-methoxy-ethoxymethyl)-pyridin-2-ylamino]-5-methyl-8H-pyrido[2,3-d]pyrimidin-7-one, 6-Bromo-8-cyclopentyl-2-[5-(2-diethylamino-ethoxy)-pyridin-2-ylamino]-5-methyl-8H-pyrido[2,3-d]pyrimidin-7-one, 6-Bromo-8-cyclopentyl-5-methyl-2-(5-pyrrolidin-1-yl-pyridin-2-ylamino)-8H-pyrido[2,3-d]pyrimidin-7-one, 6-Bromo-8-cyclopentyl-5-methyl-2-(6-methyl-5-piperazin-1-yl-pyridin-2-ylamino)-8H-pyrido[2,3-d]pyrimidin-7-one, 6-Acetyl-8-cyclopentyl-5-methyl-2-(3,4,5,6-tetrahydro-2H-[1,3′]bipyridinyl-6′-ylamino)-8H-pyrido[2,3-d]pyrimidin-7-one, 6-Acetyl-8-cyclopentyl-2-(5-diethylamino-pyridin-2-ylamino)-5-methyl-8H-pyrido[2,3-d]pyrimidin-7-one, 6-Acetyl-2-{5-[bis-(2-hydroxy-ethyl)-amino]-pyridin-2-ylamino}-8-cyclopentyl-5-methyl-8H-pyrido[2,3-d]pyrimidin-7-one, 6-Acetyl-2-[5-(2-amino-ethylamino)-pyridin-2-ylamino]-8-cyclopentyl-5-methyl-8H-pyrido[2,3-d]pyrimidin-7-one, 6-Acetyl-8-cyclopentyl-2-(5-dimethylamino-pyridin-2-ylamino)-5-methyl-8H-pyrido[2,3-d]pyrimidin-7-one, N-[6-(6-Acetyl-8-cyclopentyl-5-methyl-7-oxo-7,8-dihydro-pyrido[2,3-d]pyrimidin-2-ylamino)-pyridin-3-yl]-N-methyl-acetamide, 6-Acetyl-8-cyclopentyl-2-[5-(2-methoxy-ethoxy)-pyridin-2-ylamino]-5-methyl-8H-pyrido[2,3-d]pyrimidin-7-one, 6-Acetyl-8-cyclopentyl-2-[5-(2-methoxy-ethoxymethyl)-pyridin-2-ylamino]-5-methyl-8H-pyrido[2,3-d]pyrimidin-7-one, 6-Acetyl-8-cyclopentyl-2-[5-(2-diethylamino-ethoxy)-pyridin-2-ylamino]-5-methyl-8H-pyrido[2,3-d]pyrimidin-7-one, 6-Acetyl-8-cyclopentyl-5-methyl-2-(5-pyrrolidin-1-yl-pyridin-2-ylamino)-8H-pyrido[2,3-d]pyrimidin-7-one, 6-Acetyl-8-cyclopentyl-5-methyl-2-(6-methyl-5-piperazin-1-yl-pyridin-2-ylamino)-8H-pyrido[2,3-d]pyrimidin-7-one, 6-Acetyl-8-cyclopentyl-2-(3,4,5,6-tetrahydro-2H-[1,3′]bipyridinyl-6′-ylamino)-8H-pyrido[2,3-d]pyrimidin-7-one, 6-Acetyl-8-cyclopentyl-2-(5-morpholin-4-yl-pyridin-2-ylamino)-8H-pyrido[2,3-d]pyrimidin-7-one, 6-Acetyl-8-cyclopentyl-2-(5-diethylamino-pyridin-2-ylamino)-8H-pyrido[2,3-d]pyrimidin-7-one, 6-Acetyl-2-{5-[bis-(2-hydroxy-ethyl)-amino]-pyridin-2-ylamino}-8-cyclopentyl-8H-pyrido[2,3-d]pyrimidin-7-one, 6-Acetyl-2-{5-[bis-(2-methoxy-ethyl)-amino]-pyridin-2-ylamino}-8-cyclopentyl-8H-pyrido[2,3-d]pyrimidin-7-one, 6-Acetyl-2-[5-(2-amino-ethylamino)-pyridin-2-ylamino]-8-cyclopentyl-8H-pyrido[2,3-d]pyrimidin-7-one, 6-Acetyl-8-cyclopentyl-2-(5-dimethylamino-pyridin-2-ylamino)-8H-pyrido[2,3-d]pyrimidin-7-one, N-[6-(6-Acetyl-8-cyclopentyl-7-oxo-7,8-dihydro-pyrido[2,3-d]pyrimidin-2-ylamino)-pyridin-3-yl]-N-methyl-acetamide, 6-Acetyl-8-cyclopentyl-2-[5-(2-methoxy-ethoxy)-pyridin-2-ylamino]-8H-pyrido[2,3-d]pyrimidin-7-one, 6-Acetyl-8-cyclopentyl-2-[5-(2-methoxy-ethoxymethyl)-pyridin-2-ylamino]-8H-pyrido[2,3-d]pyrimidin-7-one, 6-Acetyl-8-cyclopentyl-2-[5-(2-diethylamino-ethoxy)-pyridin-2-ylamino]-8H-pyrido[2,3-d]pyrimidin-7-one, 6-Acetyl-8-cyclopentyl-2-(5-pyrrolidin-1-yl-pyridin-2-ylamino)-8H-pyrido[2,3-d]pyrimidin-7-one, 6-Acetyl-8-cyclopentyl-2-(6-methyl-5-piperazin-1-yl-pyridin-2-ylamino)-8H-pyrido[2,3-d]pyrimidin-7-one, 6-Bromo-8-cyclopentyl-2-[5-(2-methoxy-ethoxy)-pyridin-2-ylamino]-8H-pyrido[2,3d]pyrimidin-7-one, 6-Bromo-8-cyclopentyl-2-[5-(2-methoxy-ethylamino)-pyridin-2-ylamino]-8H-pyrido[2,3-d]pyrimidin-7-one, 2-(5-Azetidin-1-yl-pyridin-2-ylamino)-6-bromo-8-cyclopentyl-8H-pyrido[2,3-d]pyrimidin-7-one, 2-(5-Azepan-1-yl-pyridin-2-ylamino)-6-bromo-8-cyclopentyl-8H-pyrido[2,3-d]pyrimidin-7-one, N-[6-(6-Bromo-8-cyclopentyl-7-oxo-7,8-dihydro-pyrido[2,3-d]pyrimidin-2-ylamino)-pyridin-3-yl]-acetamide, 6-Bromo-8-cyclopentyl-2-(5-phenylamino-pyridin-2-ylamino)-8H-pyrido[2,3-d]pyrimidin-7-one, 6-Bromo-8-cyclopentyl-2-[5-(4-fluoro-benzylamino)-pyridin-2-ylamino]-8H-pyrido[2,3-d]pyrimidin-7-one, N-[6-(6-Bromo-8-cyclopentyl-7-oxo-7,8-dihydro-pyrido[2,3-d]pyrimidin-2-ylamino)-pyridin-3-yl]-methanesulfonamide, 6-Bromo-8-cyclopentyl-2-(5-methanesulfonyl-pyridin-2-ylamino)-8H-pyrido[2,3-d]pyrimidin-7-one, 6-Bromo-8-cyclopentyl-2-(5-phenyl-pyridin-2-ylamino)-8H-pyrido[2,3-d]pyrimidin-7-one, 6-Amino-8-cyclopentyl-2-[5-(2-methoxy-ethoxy)-pyridin-2-ylamino]-8H-pyrido[2,3-d]pyrimidin-7-one, 6-Amino-8-cyclopentyl-2-[5-(2-methoxy-ethylamino)-pyridin-2-ylamino]-8H-pyrido[2,3-d]pyrimidin-7-one, 6-Amino-2-(5-azetidin-1-yl-pyridin-2-ylamino)-8-cyclopentyl-8H-pyrido[2,3-d]pyrimidin-7-one, 6-Amino-2-(5-azepan-1-yl-pyridin-2-ylamino)-8-cyclopentyl-8H-pyrido[2,3-d]pyrimidin-7-one, N-[6-(6-Amino-8-cyclopentyl-7-oxo-7,8-dihydro-pyrido[2,3-d]pyrimidin-2-ylamino)-pyridin-3-yl]-acetamide, 6-Amino-8-cyclopentyl-2-(5-phenylamino-pyridin-2-ylamino)-8H-pyrido[2,3-d]pyrimidin-7-one, 6-Amino-8-cyclopentyl-2-[5-(4-fluoro-benzylamino)-pyridin-2-ylamino]-8H-pyrido[2,3-d]pyrimidin-7-one, N-[6-(6-Amino-8-cyclopentyl-7-oxo-7,8-dihydro-pyrido[2,3-d]pyrimidin-2-ylamino)-pyridin-3-yl]-methanesulfonamide, 6-Amino-8-cyclopentyl-2-(5-methanesulfonyl-pyridin-2-ylamino)-8H-pyrido[2,3-d]pyrimidin-7-one, 6-Amino-8-cyclopentyl-2-(5-phenyl-pyridin-2-ylamino)-8H-pyrido[2,3-d]pyrimidin-7-one, 6-Acetyl-8-cyclopentyl-2-[5-(2-methoxy-ethoxy)-pyridin-2-ylamino]-5-methyl-8H-pyrido[2,3-d]pyrimidin-7-one, 6-Acetyl-8-cyclopentyl-2-[5-(2-methoxy-ethylamino)-pyridin-2-ylamino]-5-methyl-8H-pyrido[2,3-d]pyrimidin-7-one, 6-Acetyl-2-(5-azetidin-1-yl-pyridin-2-ylamino)-8-cyclopentyl-5-methyl-8H-pyrido[2,3-d]pyrimidin-7-one, 6-Acetyl-2-(5-azepan-1-yl-pyridin-2-ylamino)-8-cyclopentyl-5-methyl-8H-pyrido[2,3-d]pyrimidin-7-one, N-[6-(6-Acetyl-8-cyclopentyl-5-methyl-7-oxo-7,8-dihydro-pyrido[2,3-d]pyrimidin-2-ylamino)-pyridin-3-yl]-acetamide, 6-Acetyl-8-cyclopentyl-5-methyl-2-(5-phenylamino-pyridin-2-ylamino)-8H-pyrido[2,3-d]pyrimidin-7-one, 6-Acetyl-8-cyclopentyl-2-[5-(4-fluoro-benzylamino)-pyridin-2-ylamino]-5-methyl-8H-pyrido[2,3-d]pyrimidin-7-one, N-[6-(6-Acetyl-8-cyclopentyl-5-methyl-7-oxo-7,8-dihydro-pyrido[2,3-d]pyrimidin-2-ylamino)-pyridin-3-yl]-methanesulfonamide, 6-Acetyl-8-cyclopentyl-2-(5-methanesulfonyl-pyridin-2-ylamino)-5-methyl-8H-pyrido[2,3-d]pyrimidin-7-one, 6-Acetyl-8-cyclopentyl-5-methyl-2-(5-phenyl-pyridin-2-ylamino)-8H-pyrido[2,3-d]pyrimidin-7-one, 6-Benzyl-8-cyclopentyl-2-[5-(2-methoxy-ethoxy)-pyridin-2-ylamino]-8H-pyrido[2,3-d]pyrimidin-7-one, 6-Benzyl-8-cyclopentyl-2-[5-(2-methoxy-ethylamino)-pyridin-2-ylamino]-8H-pyrido[2,3-d]pyrimidin-7-one, 2-(5-Azetidin-1-yl-pyridin-27ylamino)-6-benzyl-8-cyclopentyl-8H-pyrido[2,3-d]pyrimidin-7-one, 2-(5-Azepan-1-yl-pyridin-2-ylamino)-6-benzyl-8-cyclopentyl-8H-pyrido[2,3-d]pyrimidin-7-one, N-[6-(6-Benzyl-8-cyclopentyl-7-oxo-7,8-dihydro-pyrido[2,3-d]pyrimidin-2-ylamino)-pyridin-3-yl]-acetamide, 6-Benzyl-8-cyclopentyl-2-(5-phenylamino-pyridin-2-ylamino)-8H-pyrido[2,3-d]pyrimidin-7-one, 6-Benzyl-8-cyclopentyl-2-[5-(4-fluoro-benzylamino)-pyridin-2-ylamino]-8H-pyrido[2,3-d]pyrimidin-7-one, N-[6-(6-Benzyl-8-cyclopentyl-7-oxo-7,8-dihydro-pyrido[2,3-d]pyrimidin-2-ylamino)-pyridin-3-yl]-methanesulfonamide, 6-Benzyl-8-cyclopentyl-2-(5-methanesulfonyl-pyridin-2-ylamino)-8H-pyrido[2,3-d]pyrimidin-7-one, 6-Benzyl-8-cyclopentyl-2-(5-phenyl-pyridin-2-ylamino)-8H-pyrido[2,3-d]pyrimidin-7-one, 8-Cyclopentyl-6-hydroxymethyl-2-[5-(2-methoxy-ethoxy)-pyridin-2-ylamino]-8H-pyrido[2,3-d]pyrimidin-7-one, 8-Cyclopentyl-6-hydroxymethyl-2-[5-(2-methoxy-ethylamino)-pyridin-2-ylamino]-8H-pyrido[2,3-d]pyrimidin-7-one, 2-(5-Azetidin-1-yl-pyridin-2-ylamino)-8-cyclopentyl-6-hydroxymethyl-8H-pyrido[2,3-d]pyrimidin-7-one, 2-(5-Azepan-1-yl-pyridin-2-ylamino)-8-cyclopentyl-6-hydroxymethyl-8H-pyrido[2,3-d]pyrimidin-7-one, N-[6-(8-Cyclopentyl-6-hydroxymethyl-7-oxo-7,8-dihydro-pyrido[2,3-d]pyrimidin-2-ylamino)-pyridin-3-yl]-acetamide, 8-Cyclopentyl-6-hydroxymethyl-2-(5-phenylamino-pyridin-2-ylamino)-8H-pyrido[2,3-d]pyrimidin-7-one, 8-Cyclopentyl-2-[5-(4-fluoro-benzylamino)-pyridin-2-ylamino]-6-hydroxymethyl-8H-pyrido[2,3-d]pyrimidin-7-one, N-[6-(8-Cyclopentyl-6-hydroxymethyl-7-oxo-7,8-dihydro-pyrido[2,3-d]pyrimidin-2-ylamino)-pyridin-3-yl]-methanesulfonamide, 8-Cyclopentyl-6-hydroxymethyl-2-(5-methanesulfonyl-pyridin-2-ylamino)-8H-pyrido[2,3-d]pyrimidin-7-one, 8-Cyclopentyl-6-hydroxymethyl-2-(5-phenyl-pyridin-2-ylamino)-8H-pyrido[2,3-d]pyrimidin-7-one, 8-Cyclopentyl-6-ethyl-2-[5-(2-methoxy-ethoxy)-pyridin-2-ylamino]-8H-pyrido[2,3-d]pyrimidin-7-one, 8-Cyclopentyl-6-ethyl-2-[5-(2-methoxy-ethylamino)-pyridin-2-ylamino]-8H-pyrido[2,3-d]pyrimidin-7-one, 2-(5-Azetidin-1-yl-pyridin-2-ylamino)-8-cyclopentyl-6-ethyl-8H-pyrido[2,3-d]pyrimidin-7-one, 2-(5-Azepan-1-yl-pyridin-2-ylamino)-8-cyclopentyl-6-ethyl-8H-pyrido[2,3-d]pyrimidin-7-one, N-[6-(8-Cyclopentyl-6-ethyl-7-oxo-7,8-dihydro-pyrido[2,3-d]pyrimidin-2-ylamino)-pyridin-3-yl]-acetamide, 8-Cyclopentyl-6-ethyl-2-(5-phenylamino-pyridin-2-ylamino)-8H-pyrido[2,3-d]pyrimidin-7-one, 8-Cyclopentyl-6-ethyl-2-[5-(4-fluoro-benzylamino)-pyridin-2-ylamino]-8H-pyrido[2,3-d]pyrimidin-7-one, N-[6-(8-Cyclopentyl-6-ethyl-7-oxo-7,8-dihydro-pyrido[2,3-d]pyrimidin-2-ylamino)-pyridin-3-yl]-methanesulfonamide, 8-Cyclopentyl-6-ethyl-2-(5-methanesulfonyl-pyridin-2-ylamino)-8H-pyrido[2,3-d]pyrimidin-7-one, 8-Cyclopentyl-6-ethyl-2-(5-phenyl-pyridin-2-ylamino)-8H-pyrido[2,3-d]pyrimidin-7-one, 6-Bromo-8-cyclopentyl-2-[5-(piperazine-1-carbonyl)-pyridin-2-ylamino]-8H-pyrido[2,3-d]pyrimidin-7-one, 6-Bromo-8-cyclopentyl-2-[5-(3,5-dimethyl-piperazine-1-carbonyl)-pyridin-2-ylamino]-8H-pyrido[2,3-d]pyrimidin-7-one, 2-[5-(3-Amino-pyrrolidine-1-carbonyl)-pyridin-2-ylamino]-6-bromo-8-cyclopentyl-8H-pyrido[2,3-d]pyrimidin-7-one, 6-Bromo-8-cyclopentyl-2-[5-(morpholine-4-carbonyl)-pyridin-2-ylamino]-8H-pyrido[2,3-d]pyrimidin-7-one, 6-Bromo-8-cyclopentyl-5-methyl-2-[5-(piperazine-1-carbonyl)-pyridin-2-ylamino]-8H-pyrido[2,3-d]pyrimidin-7-one, 6-Bromo-8-cyclopentyl-2-[5-(3,5-dimethyl-piperazine-1-carbonyl)-pyridin-2-ylamino]-5-methyl-8H-pyrido[2,3-d]pyrimidin-7-one, 2-[5-(3-Amino-pyrrolidine-1-carbonyl)-pyridin-2-ylamino]-6-bromo-8-cyclopentyl-5-methyl-8H-pyrido[2,3-d]pyrimidin-7-one, 6-Bromo-8-cyclopentyl-5-methyl-2-[5-(morpholine-4-carbonyl)-pyridin-2-ylamino]-8H-pyrido[2,3-d]pyrimidin-7-one, 6-Acetyl-8-cyclopentyl-5-methyl-2-[5-(piperazine-1-carbonyl)-pyridin-2-ylamino]-8H-pyrido[2,3-d]pyrimidin-7-one, 6-Acetyl-8-cyclopentyl-2-[5-(3,5-dimethyl-piperazine-1-carbonyl)-pyridin-2-ylamino]-5-methyl-8H-pyrido[2,3-d]pyrimidin-7-one, 6-Acetyl-2-[5-(3-amino-pyrrolidine-1-carbonyl)-pyridin-2-ylamino]-8-cyclopentyl-5-methyl-8H-pyrido[2,3-d]pyrimidin-7-one, 6-Acetyl-8-cyclopentyl-5-methyl-2-[5-(morpholine-4-carbonyl)-pyridin-2-ylamino]-8H-pyrido[2,3-d]pyrimidin-7-one, 8-Cyclopentyl-6-ethyl-2-[5-(piperazine-1-carbonyl)-pyridin-2-ylamino]-8H-pyrido[2,3-d]pyrimidin-7-one, 8-Cyclopentyl-2-[5-(3,5-dimethyl-piperazine-1-carbonyl)-pyridin-2-ylamino]-6-ethyl-8H-pyrido[2,3-d]pyrimidin-7-one, 2-[5-(3-Amino-pyrrolidine-1-carbonyl)-pyridin-2-ylamino]-8-cyclopentyl-6-ethyl-8H-pyrido[2,3-d]pyrimidin-7-one, 8-Cyclopentyl-6-ethyl-2-[5-(morpholine-4-carbonyl)-pyridin-2-ylamino]-8H-pyrido[2,3-d]pyrimidin-7-one, 6-Bromo-8-cyclopentyl-2-[5-(piperazine-1-sulfonyl)-pyridin-2-ylamino]-8H-pyrido[2,3-d]pyrimidin-7-one, 6-Bromo-8-cyclopentyl-2-[5-(morpholine-4-sulfonyl)-pyridin-2-ylamino]-8H-pyrido[2,3-d]pyrimidin-7-one, 2-[5-(3-Amino-pyrrolidine-1-sulfonyl)-pyridin-2-ylamino]-6-bromo-8-cyclopentyl-8H-pyrido[2,3-d]pyrimidin-7-one, 6-Bromo-8-cyclopentyl-2-[5-(3,5-dimethyl-piperazine-1-sulfonyl)-pyridin-2-ylamino]-8H-pyrido[2,3-d]pyrimidin-7-one, 6-Bromo-8-cyclopentyl-5-methyl-2-[5-(piperazine-1-sulfonyl)-pyridin-2-ylamino]-8H-pyrido[2,3-d]pyrimidin-7-one, 6-Bromo-8-cyclopentyl-5-methyl-2-[5-(morpholine-4-sulfonyl)-pyridin-2-ylamino]-8H-pyrido[2,3-d]pyrimidin-7-one, 2-[5-(3-Amino-pyrrolidine-1-sulfonyl)-pyridin-2-ylamino]-6-bromo-8-cyclopentyl-5-methyl-8H-pyrido[2,3-d]pyrimidin-7-one, 6-Bromo-8-cyclopentyl-2-[5-(3,5-dimethyl-piperazine-1-sulfonyl)-pyridin-2-ylamino]-5-methyl-8H-pyrido[2,3-d]pyrimidin-7-one, 8-Cyclopentyl-6-ethyl-2-[5-(piperazine-1-sulfonyl)-pyridin-2-ylamino]-8H-pyrido[2,3-d]pyrimidin-7-one, 8-Cyclopentyl-6-ethyl-2-[5-(morpholine-4-sulfonyl)-pyridin-2-ylamino]-8H-pyrido[2,3-d]pyrimidin-7-one, 2-[5-(3-Amino-pyrrolidine-1-sulfonyl)-pyridin-2-ylamino]-8-cyclopentyl-6-ethyl-8H-pyrido[2,3-d]pyrimidin-7-one, 8-Cyclopentyl-2-[5-(3,5-dimethyl-piperazine-1-sulfonyl)-pyridin-2-ylamino]-6-ethyl-8H-pyrido[2,3-d]pyrimidin-7-one, 6-Acetyl-8-cyclopentyl-5-methyl-2-[5-(piperazine-1-sulfonyl)-pyridin-2-ylamino]-8H-pyrido[2,3-d]pyrimidin-7-one, 6-Acetyl-8-cyclopentyl-5-methyl-2-[5-(morpholine-4-sulfonyl)-pyridin-2-ylamino]-8H-pyrido[2,3-d]pyrimidin-7-one, 6-Acetyl-2-[5-(3-amino-pyrrolidine-1-sulfonyl)-pyridin-2-ylamino]-8-cyclopentyl-5-methyl-8H-pyrido[2,3-d]pyrimidin-7-one, 6-Acetyl-8-cyclopentyl-2-[5-(3,5-dimethyl-piperazine-1-sulfonyl)-pyridin-2-ylamino]-5-methyl-8H-pyrido[2,3-d]pyrimidin-7-one, and 6-Acetyl-8-cyclopentyl-5-methyl-2-([1,6]naphthyridin-2-ylamino)-8H-pyrido[2,3-d]pyrimidin-7-one, 6-Acetyl-8-cyclopentyl-2-[5-(1,1-dioxo-116-thiomorpholin-4-yl)-pyridin-2-ylamino]-5-methyl-8H-pyrido[2,3-d]pyrimidin-7-one, 8-Cyclopentyl-6-hydroxymethyl-5-methyl-2-(5-piperazin-1-yl-pyridin-2-ylamino)-8H-pyrido[2,3-d]pyrimidin-7-one, 6-Acetyl-2-(3-chloro-5-piperazin-1-yl-pyridin-2-ylamino)-8-cyclopentyl-5-methyl-8H-pyrido[2,3-d]pyrimidin-7-one, 4-[6-Acetyl-5-methyl-7-oxo-2-(pyridin-2-ylamino)-7H-pyrido[2,3-d]pyrimidin-8-yl]-cyclohexanecarboxylic acid, 4-[6-Acetyl-2-(5-dimethylamino-pyridin-2-ylamino)-5-methyl-7-oxo-7H-pyrido[2,3-d]pyrimidin-8-yl]-cyclohexanecarboxylic acid, 6-Bromo-8-cyclopentyl-5-methyl-2-[5-(piperazine-1-sulfonyl)-pyridin-2-ylamino]-8H-pyrido[2,3-d]pyrimidin-7-one, 6-(8-Cyclopentyl-6-ethyl-7-oxo-7,8-dihydro-pyrido[2,3-d]pyrimidin-2-ylamino)-3-piperazin-1-yl-pyridine-2-carboxylic acid, 2-(6-Acetyl-5-piperazin-1-yl-pyridin-2-ylamino)-8-cyclopentyl-6-ethyl-8H-pyrido[2,3-d]pyrimidin-7-one, 3-{2-[6-(8-Cyclopentyl-6-ethyl-7-oxo-7,8-dihydro-pyrido[2,3-d]pyrimidin-2-ylamino)-pyridin-3-yloxy]-ethoxy}-propionic acid, [6-(8-Cyclopentyl-6-ethyl-7-oxo-7,8-dihydro-pyrido[2,3-d]pyrimidin-2-ylamino)-pyridin-3-yloxy]-acetic acid, 8-Cyclopentyl-2-(5-{2-[2-(5-methyl-pyridin-2-yl)-ethoxy]-ethoxy}-pyridin-2-ylamino)-8H-pyrido[2,3-d]pyrimidin-7-one, 2-[5-(3-Benzenesulfonyl-propoxy)-pyridin-2-ylamino]-8-cyclopentyl-8H-pyrido[2,3-d]pyrimidin-7-one, 8-Cyclopentyl-6-ethyl-2-{5-[2-(2-methoxy-ethoxy)-ethoxy]-pyridin-2-ylamino}-8H-pyrido[2,3-d]pyrimidin-7-one, 8-Cyclopentyl-2-(5-{[3-(3,5-dimethyl-piperazin-1-yl)-propyl]-methyl-amino}-pyridin-2-ylamino)-8H-pyrido[2,3-d]pyrimidin-7-one, 8-Cyclopentyl-2-{5-[(3-imidazol-1-yl-propyl)-methyl-amino]-pyridin-2-ylamino}-8H-pyrido[2,3-d]pyrimidin-7-one, 6-Acetyl-5-methyl-2-(5-methyl-pyridin-2-ylamino)-8-piperidin-4-yl-8H-pyrido[2,3-d]pyrimidin-7-one, and 6-Acetyl-2-[5-(3,4-dihydroxy-pyrrolidin-1-yl)-pyridin-2-ylamino]-8-methoxymethyl-5-methyl-8H-pyrido[2,3-d]pyrimidin-7-one.

For example, the compound is selected from a group consisting of:

8-Cyclopentyl-2-(pyridin-2-ylamino)-8H-pyrido[2,3-d]pyrimidin-7-one, 6-Bromo-8-cyclopentyl-2-(5-piperazin-1-yl-pyridin-2-ylamino)-8H-pyrido [2,3-d]pyrimidin-7-one hydrochloride, 8-Cyclopentyl-6-ethyl-2-(5-piperazin-1-yl-pyridin-2-ylamino)-8H-pyrido[2,3-d]pyrimidin-7-one hydrochloride, 8-Cyclopentyl-7-oxo-2-(5-piperazin-1-yl-pyridin-2-ylamino)-7,8-dihydro-pyrido[2,3-d]pyrimidine-6-carboxylic acid ethyl ester hydrochloride, 6-Amino-8-cyclopentyl-2-(5-piperazin-1-yl-pyridin-2-ylamino)-8H-pyrido[2,3-d]pyrimidin-7-one hydrochloride, 6-Bromo-8-cyclopentyl-2-[5-((R)-methy-1-pyrrolidin-2-yl)-pyridin-2-ylamino]-8H-pyrido[2,3-d]pyrimidin-7-one hydrochloride, 6-Bromo-8-cyclohexyl-2-(pyridin-2-yl-amino)-8H-pyrido[2,3-d]pyrimidin-7-one, 6-Bromo-8-cyclopentyl-2-methyl-8H-pyrido[2,3-d]pyrimidin-7-one, 6-Bromo-8-cyclopentyl-5-methyl-2-(5-piperazin-1-yl-pyridin-2-ylamino)-8H-pyrido[2,3-d]pyrimidin-7-one, 8-Cyclopentyl-6-fluoro-2-(5-piperazin-1-yl-pyridin-2-ylamino)-8H-pyrido[2,3-d]pyrimidin-7-one hydrochloride, 8-Cyclopentyl-6-methyl-2-(5-piperazin-1-yl-pyridin-2-ylamino)-8H-pyrido[2,3-d]pyrimidin-7-one hydrochloride, 8-Cyclopentyl-6-isobutoxy-2-(5-piperazin-1-yl-pyridin-2-ylamino)-8H-pyrido[2,3-d]pyrimidin-7-one hydrochloride, 6-Benzyl-8-cyclopentyl-2-(5-piperazin-1-yl-pyridin-2-ylamino)-8H-pyrido[2,3-d]pyrimidin-7-one hydrochloride, 8-Cyclopentyl-6-hydroxymethyl-2-(5-piperazin-1-yl-pyridin-2-ylamino)-8H-pyrido[2,3-d]pyrimidin-7-one hydrochloride, 2-[5-(4-tert-Butoxycarbonyl-piperazin-1-yl)-pyridin-2-ylamino]-8-cyclopentyl-5-methyl-7-oxo-7,8-dihydro-pyrido[2,3-d]pyrimidine-6-carboxylic acid ethyl ester, 6-Acetyl-8-cyclopentyl-2-(5-piperazin-1-yl-pyridin-2-ylamino)-8H-pyrido[2,3-d]pyrimidin-7-one, 6-Acetyl-8-cyclopentyl-5-methyl-2-(5-piperazin-1-yl-pyridin-2-ylamino)-8H-pyrido[2,3-d]pyrimidin-7-one, 6-Bromo-8-cyclopentyl-5-methyl-2-(pyridin-2-ylamino)-8H-pyrido[2,3-d]pyrimidin-7-one, and 6-Bromo-8-cyclopentyl-2-(pyridin-2-ylamino)-8H-pyrido[2,3-d]pyrimidin-7-one.

For example, the compound is 6-Acetyl-8-cyclopentyl-5-methyl-2-(5-piperazin-1-yl-pyridin-2-ylamino)-8H-pyrido[2,3-d] pyrimidin-7-one.

For example, the compound may be a substituted 2-aminopyridines useful in treating cell proliferative disorders, which are potent inhibitors of cyclin-dependent kinases 4 (cdk4). For example, the compound may be recorded in U.S. Pat. No. 6,936,612B2 and/or US20030149001A1.

In present application, the medicinal product may be a combination of the HER2 inhibitor in present application and the CDK inhibitor in present application. For example, the HER2 inhibitor may simultaneously appear with the CDK inhibitor on the prescription for treating cancer. For example, a specification/label of the HER2 inhibitor in present application reads recommending to take the CDK inhibitor or CDK4 inhibitor before, after or simultaneously. For another example, a specification/label of the CDK inhibitor in present application reads recommending to take the HER2 inhibitor in present application before, after or simultaneously.

In present application, the medicinal product may be a composition. For example, the HER2 inhibitor and the CDK inhibitor may be not mixed with each other in the composition. For example, the HER2 inhibitor and the CDK inhibitor may be packaged in different isolated packages and/or containers.

In present application, the compositions typically may be sterile and stable under the conditions of manufacture and storage. The composition may be formulated as a solution, microemulsion, liposome, or other ordered structure suitable to high drug concentration. The carrier may be a solvent or dispersion medium containing, for example, water, ethanol, polyol (for example, glycerol, propylene glycol, and liquid polyethylene glycol, and the like), and suitable mixtures thereof. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions may be brought about by including in the composition an agent that delays absorption, for example, monostearate salts and gelatin.

For example, the composition may include other compounds, drugs, and/or agents used for the treatment of cancer. Such compounds, drugs, and/or agents can include, for example, chemotherapy drugs, small molecule drugs or antibodies that stimulate the immune response to a given cancer. In some instances, the composition may include, for example, one or more of the agents listed in the section on combination therapies. For example, the combination therapy can include at least one other anti-cancer and/or T-cell stimulating (e.g., activating) agent.

For example, the medicinal product may further comprise fulvestrant.

For example, the HER2 inhibitor, the CDK inhibitor and fulvestrant may be not mixed with each other in the composition. For example, the HER2 inhibitor, the CDK inhibitor and the fulvestrant may be packaged in different isolated packages and/or containers.

### Use and Methods

In another aspect, the present application provides the HER2 inhibitor of the present application for the use of preventing, alleviating or treating tumor or inhibiting tumor growth in combination with the CDK inhibitor of the present application.

In another aspect, the present application provides the medical product of the present application, for the use of preventing, alleviating or treating tumor or inhibiting tumor growth.

In another aspect, the present application provides a use of the HER2 inhibitor of the present application in combination with the CDK inhibitor of the present application in the preparation of a medicament for preventing, alleviating or treating tumor or inhibiting tumor growth in a subject in need thereof.

For example, the present application may provide a use of the medical product of the present application in the preparation of a medicament for preventing, alleviating or treating tumor or inhibiting tumor growth in a subject in need thereof.

In another aspect, the present application provides a method of preventing, alleviating or treating tumor or inhibiting tumor growth in a subject in need thereof, comprising: administrating the HER2 inhibitor of the present application in combination with the CDK inhibitor of the present application to the subject.

For example, the present application may provide a method of preventing, alleviating or treating tumor or inhibiting tumor growth in a subject in need thereof, comprising: administrating the composition of the present application to the subject.

For example, the method may further comprise administering to the subject (c) an effective amount of fulvestrant.

For example, the subject may be not responsive to a conventional therapy for HER2-related tumor. For example, the conventional therapy for HER2-related tumor may comprise administrating drugs which is specific for targeting HER2. For example, may comprise HER2 antigen binding protein (for example, anti-HER2 antibody), the conjugate thereof and/or HER2 specific inhibitors. For example, the conventional therapy for HER2-related tumor may comprise administrating HER2-ADC, MBC hormone, Taxane, pyrotinib, neratinib, tucatinib, trastuzumab and/or pertuzumab. For example, the conventional therapy for HER2-related tumor may comprise administrating drugs which is universal for treating tumor. For example, may comprise any available chemotherapy drugs. For example, the conventional therapy for HER2-related tumor may comprise administrating docetaxel, capecitabine and/or lapatinib.

In present application, the not responsive may refer to a syndrome of the tumor of the subject has not been alleviated significantly after administrated with the conventional therapy for HER2-related tumor. For example, the syndrome may comprise a decrease of the volume of a tumor. For example, the syndrome may comprise an extension of the OS, ORR and/or PFS.

In the present application, the subject in need of may have been failed in a conventional therapy for HER2-related tumor, and the conventional therapy for HER2-related tumor may comprise administrating trastuzumab, MBC hormone and/or Taxane.

For example, the subject in need of may have failed in a conventional therapy for HER2-related tumor. For example, the conventional therapy for HER2-related tumor may comprise administrating Trastuzumab, HER2 TKI and HER2 ADC. For example, the median number of prior lines of the conventional HER2 target therapy among the subject in need of may be 2 (range: 1-12).

For example, the subject in need thereof may have HER2-positive metastatic breast cancer whose disease has progressed after treatment with trastuzumab and/or a taxane.

For example, the subject in need thereof may have been treated with prior hormonal treatment. For example, the hormonal treatment may comprise administrating drugs blocking estrogen receptors. For example, the hormonal treatment may comprise treatment with Tamoxifen and/or Toremifene. For example, the taxane may comprise Paclitaxel (Taxol) and docetaxel (Taxotere).

For example, the subject in need thereof may have been treated with the hormonal treatment first, and then been treated with trastuzumab and/or a taxane. For another example, the subject in need thereof may have been treated with trastuzumab and/or a taxane first, and then been treated with the hormonal treatment.

In the present application, the tumor may comprise a solid tumor. For example, the tumor may comprise metastatic tumor, early tumor and/or locally advanced tumor. For example, the tumor may comprise HER2 positive tumor and/or HER2 low-expression tumor.

For example, the tumor may comprise a breast cancer and/or a gastric cancer. For example, the breast cancer may comprise HER2 positive breast cancer and/or HER2 low-expression breast cancer. For example, the breast cancer may comprise early breast cancer, locally advanced breast cancer and/or metastatic breast cancer. For example, the gastric cancer may comprise early gastric cancer, locally advanced gastric cancer and/or metastatic gastric cancer. For example, the subject in need thereof may have histologically or cytologically proven diagnosis of HER2-positive adenocarcinoma of the breast at the time of diagnosing locally advanced unresectable or metastatic disease.

For example, the tumor may be selected from the group consisting of cancers of the breast, ovary, cervix, prostate, testis, esophagus, stomach, skin, lung, bone, colon, pancreas, thyroid, biliary passages, buccal cavity and pharynx (oral), lip, tongue, mouth, pharynx, small intestine, colon-rectum, large intestine, rectum, brain and central nervous system, glioblastoma, neuroblastoma, keratoacanthoma, epidermoid carcinoma, large cell carcinoma, adenocarcinoma, adenocarcinoma, adenoma, adenocarcinoma, follicular carcinoma, undifferentiated carcinoma, papillary carcinoma, seminoma, melanoma, Sarcoma, bladder carcinoma, liver carcinoma, kidney carcinoma, myeloid disorders, lymphoid disorders, Hodgkin's, hairy cells, and leukemia.

For example, the breast cancer may be HR negative (HR⁻) or HR positive (HR⁺) breast cancer. For example, the cancer may be HR⁻HER2-positive positive breast cancer. For another example, the cancer may be HR⁺HER2-positive positive breast cancer.

In present application, the subject in need thereof may be treated with the HER2 inhibitor at scheduled regimen until progressive disease, unacceptable toxicity or withdrawal of informed consent whichever comes first.

The HER2 inhibitor of present application may be administered by the same route of administration or by different routes of administration. For example, the HER2 inhibitor may be administrated by intravenous administration.

For example, the HER2 inhibitor may be administrated as a 90-minutes intravenous infusion for the initial dose. For example, the HER2 inhibitor may be administrated shorted to a 60-minutes intravenous infusion for subsequent doses.

In present application, a cycle may be defined as 28 days for Q2W (once for two weeks) dosing and 21 days for Q3W (once for three weeks) dosing.

For example, the HER2 inhibitor may be administrated to the subject in need at a dose of about 1 mg/kg to about 30 mg/kg (for example, about at least 1 mg/kg, about at least 1.5 mg/kg, about at least 2 mg/kg, about at least 2.5 mg/kg, about at least 5 mg/kg, about at least 6 mg/kg, about at least 7 mg/kg, about at least 8 mg/kg, about at least 9 mg/kg, about at least 10 mg/kg, about at least 15 mg/kg, about at least 20 mg/kg, about at least 25 mg/kg or about at least 30 mg/kg). For example, the HER2 inhibitor may be administrated to the subject in need at a dose of about 10 mg/kg, at a dose of about 5 mg/kg, and at a dose of about 2.5 mg/kg for the continuous three times respectively.

For example, the HER2 inhibitor may be administrated to the subject in need every two weeks. For example, the HER2 inhibitor may be administrated to the subject in need with 20 mg/kg loading on Days 1 and 8 of Cycle 1.

For example, the HER2 inhibitor may be administrated to the subject in need within ten days. For example, the HER2 inhibitor may be administrated to the subject on DAY10, DAY3 and DAY10.

For example, the CDK inhibitor may be formulated for oral use.

For example, the CDK inhibitor may be administrated to the subject in need at a dose of 80 mg to 150 mg (for example, a dose of 80 mg/day, a dose of 90 mg/day, a dose of 100 mg/day, a dose of 110 mg/day, a dose of 115 mg/day, a dose of 120 mg/day, a dose of 125 mg/day, a dose of 130 mg/day, a dose of 135 mg/day, a dose of 140 mg/day, or a dose of 150 mg/day).

For example, the CDK inhibitor may be administrated to the subject in need at a dose of about 10 mg/kg to about 50 mg/kg (for example, about at least 10 mg/kg, about at least 15 mg/kg, about at least 20 mg/kg, about at least 25 mg/kg, about at least 30 mg/kg, about at least 35 mg/kg, about at least 40 mg/kg, about at least 45 mg/kg, or about at least 50 mg/kg). For example, the CDK inhibitor may be administrated to the subject in need at a dose of about 40 mg/kg, at a dose of about 20 mg/kg, and at a dose of about 10 mg/kg for the continuous three times respectively.

For example, the CDK inhibitor may be administrated to the subject in need every day for three weeks. For example, the CDK inhibitor may be administrated to the subject in need continuously for three weeks and followed by 1 week off.

For example, the CDK inhibitor may be administrated to the subject in need for at least two weeks. For example, the CDK inhibitor may be administrated to the subject on DAY0-6; DAY7-13 and DAY14 and later.

For example, the fulvestrant in the medicinal product may be formulated for intramuscular injection.

For example, the fulvestrant in the medicinal product may be administrated to the subject in need at a dose of 400 mg to 600 mg (for example, a dose of 400 mg, a dose of 450 mg, a dose of 500 mg, a dose of 550 mg or a dose of 600 mg).

For example, the fulvestrant in the medicinal product may be administrated to the subject in need on Days 1 and 15 of Cycle 1, and then on Day 1 of each subsequent 28-day cycle.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

HER2 inhibitor was used in the example to represent said HER2 inhibitor in present application. HER2 inhibitor targets the distinct extracellular domain 2 (pertuzumab binding site) and extracellular domain 4 (trastuzumab binding site) of HER2. It blocks both ligand-dependent and ligand-independent HER2 signaling pathway. HER2 inhibitor comprises a first light chain, a second light chain, a first heavy chain and a second heavy chain. And the first light chain and the second light chain has an identical amino acid sequence set forth as SEQ ID NO.1; the variable region of the first heavy chain and the second heavy chain are set forth as SEQ ID NO.13 and SEQ ID NO.14, respectively. And HER2 inhibitor is a IgG1 antibody.

### Example 1 Study Design

This is an open-label, multicenter, dose-escalation and parallel-group expansion Phase 1b/2 clinical trial to evaluate the efficacy, safety and tolerability of HER2 inhibitor in combination with CDK inhibitor with or without fulvestrant in women or male with HER2-positive metastatic breast cancer whose disease has progressed after treatment with trastuzumab and a taxane, at the minimum in any breast cancer disease setting. Patients could have received one prior hormonal treatment for MBC.

Eligible patients must have histologically or cytologically proven diagnosis of HER2-positive adenocarcinoma of the breast at the time of diagnosing locally advanced unresectable or metastatic disease. HER2 status will be confirmed in a central laboratory with use of archival paraffin-embedded tumor tissue. Tumor HER2 status will be considered positive if the central laboratory reports Grade 3+ staining intensity (on a scale of 0 to 3+) by means of IHC analysis or gene amplification by FISH.

Dose escalation will be performed at the following 2 dose levels:
Dose level 1
Patients with HR⁻/HER2-positive MBC will receive:
HER2 inhibitor 20 mg/kg IV Q2W (with 20 mg/kg loading on Days 1 and 8 of Cycle 1)+CDK inhibitor 100 mg/day orally for 3 weeks followed by 1 week off,
Patients with HR⁺/HER2-positive MBC will receive:
HER2 inhibitor 20 mg/kg IV Q2W (with 20 mg/kg loading on Days 1 and 8 of Cycle 1)+CDK inhibitor 100 mg/day orally for 3 weeks followed by 1 week off+fulvestrant 500 mg intramuscularly on Days 1 and 15 of Cycle 1, and then on Day 1 of each subsequent 28-day cycle;
Dose level 2
Patients with HR⁻/HER2-positive MBC will receive:
HER2 inhibitor 20 mg/kg IV Q2W (with 20 mg/kg loading on Days 1 and 8 of Cycle 1)+CDK inhibitor 125 mg/day orally for 3 weeks followed by 1 week off,
Patients with HR⁺/HER2-positive MBC will receive:
HER2 inhibitor 20 mg/kg IV Q2W (with 20 mg/kg loading on Days 1 and 8 of Cycle 1)+CDK inhibitor 125 mg/day orally for 3 weeks followed by 1 week off+fulvestrant 500 mg intramuscularly on Days 1 and 15 of Cycle 1, and then on Day 1 of each subsequent 28-day cycle;

The dose escalation phase uses a mTPI-2 (modified toxicity probability interval) design. 3˜6 subjects will be enrolled at each treatment cohort. The decision to escalate to the next dose level will be based on safety assessments after all subjects of a treatment cohort have reached Day 28 (dose limiting toxicity observation period). A scientific monitoring committee (SMC) will be responsible for decisions on dose escalation, determination of combination maximum tolerated dose (MTD) or recommended phase 2 dose (RP2D) and will be established before first patient dose. SMC may decide to explore higher, lower or interim dose levels based on available safety and efficacy data from HER2 inhibitor program. Once the combination MTD or RP2D has been established, the study will enter the parallel-group expansion phase.

In parallel-group expansion phase, 30 HR⁻/HER2-positive and 30 HR⁺/HER2-positive MBC patients will be enrolled and treated by combination RP2Ds. Patients with HR⁻/HER2-positive MBC will receive HER2 inhibitor in combination with CDK inhibitor and patients with HR⁺/HER2-positive MBC will receive triple combination of HER2 INHIBITOR, CDK inhibitor and fulvestrant.

After completion of the initial parallel-group expansion, further expansion in HR⁻/HER2-positive, HR⁺/HER2-positive or further expansion in HER2-intermediate or low expressing (defined as HER2 IHC1+ or 2+ AND FISH-negative) MBC patients may be added. Protocol amendment will be issued for further expansion after the approval from applicable committees.

Patients must have at least one measurable disease at baseline from non-irradiated region or progressed within a previous radiation field. Patients will undergo tumor assessment every 8 weeks for the first 12 months and then every 12 weeks, calculated from the date of first trial treatment. Tumor assessment will continue until progressive disease according to RECIST 1.1, initiation of new anti-cancer therapy, death, or withdrawal of consent, whichever occurs first.

Patients will continue to receive assigned trial treatment until progressive disease according to RECIST 1.1, clinical deterioration, unacceptable toxicity, initiation of new anti-cancer therapy, death, or withdrawal of consent, whichever comes first. Should CDK inhibitor, HER2 inhibitor or fulvestrant related toxicity mandate discontinuation of perspective trial treatment, patients can continue to receive other study drugs.

Each subject enrolled will have a screening period (−21 to −1 day prior to first trial treatment), a treatment period, an end-of-treatment (EOT) visit, a 30-day safety follow-up visit, a 84-day safety follow-up visit and an overall survival follow-up. The study will end at 12 months after all subjects complete the last dose of trial treatment.

### Example 2 Introduction of Study

### Study Object

Primary Objective

Dose Escalation Phase
To determine MTD or RP2D of:
HER2 inhibitor in combination with CDK inhibitor in HR⁻/HER2-positive MBC;
and
HER2 inhibitor in combination with CDK inhibitor and fulvestrant in HR⁺/HER2-positive MBC;

Parallel-Group Expansion Phase
To evaluate anti-tumor activity of HER2 inhibitor in combination with CDK inhibitor in HR⁻/HER2-positive MBC;
To evaluate anti-tumor activity of HER2 inhibitor in combination with CDK inhibitor and fulvestrant in HR⁺/HER2-positive MBC;

Secondary Objectives
To assess safety and tolerability of HER2 inhibitor in combination with CDK inhibitor with or without fulvestrant;
To characterize pharmacokinetics of HER2 INHIBITOR;
To evaluate immunogenicity of HER2 INHIBITOR;

Exploratory Objectives
To explore relationship between HER2 inhibitor drug exposure and efficacy/safety findings;
To explore alterations in genes, proteins, and ribonucleic acids (RNAs) relevant to the cell cycle, drug targets, tumor sensitivity and/or resistance.

### Study Endpoints

Primary Endpoint

Dose Escalation Phase
Dose limiting toxicity (DLT);

Parallel-Group Expansion Phase
Objective response rate as assessed by the investigator according to RECIST 1.1;

Secondary Endpoints
Duration of response (DOR) and time to response (TTR);
6- and 12-months PFS and OS rates;
Clinical benefit rate (CBR), defined as proportion of complete response (CR), partial response (PR) or stable disease (SD)≥24 weeks; disease control rate (DCR), defined as proportion of CR, PR or SD;
Incidence and severity (as graded by CTCAE v5.0), seriousness and relationship to the trial treatments, abnormal findings on any laboratory test and physical examination. All cardiac AEs occurring during the study and up to 12 months after last HER2 inhibitor drug administration must be reported irrespective of causal relationship (related or unrelated) or severity (serious or non-serious);
PK parameters of HER2 inhibitor derived from population PK analysis, including but not limited to AUC, Cₘₐₓ, Cₘᵢₙ, CLand T_{1/2};
Status (positive or negative) and serum titers of anti-HER2 inhibitor antibody and neutralizing capacity;

Exploratory Endpoints
Tumor tissue biomarkers, including genes (eg, HER2 mutation, copy number of HER2, CCND1 and CDKN2A, PIK3CA mutation), proteins (eg, Ki67, pRb, CCNE1) and RNA expression (eg, cdk4, cdk6).

### Study Population

### Inclusion Criteria

101. Signed informed consent;

102. Postmenopausal female or male subjects 18 years of age or older. Postmenopausal women are defined by at least one of the following criteria:
Age ≥60 years;
Age <60 years and cessation of regular menses for at least 12 consecutive months with no alternative pathological or physiological cause; and serum estradiol and FSH level within the laboratory's reference range for postmenopausal females;
Documented bilateral oophorectomy;
Medically confirmed ovarian failure;

103. Histologically or cytologically proven diagnosis of breast cancer with evidence of metastatic or locally advanced disease not amenable to resection or radiation therapy with curative intent;

104. HER2-positive tumor based on tumor tissue collected at the time of diagnosing locally advanced unresectable or metastatic disease. HER2-positivity is defined as IHC 3+ and/or HER2 gene-amplified by FISH. HER2 status determined by local laboratory can be used to determine eligibility for this study however tissue samples MUST be submitted to a Sponsor-designated central laboratory to confirm HER2 status;

105. Prior treatment for breast cancer in the adjuvant, locally advanced unresectable or metastatic setting must include a taxane and trastuzumab;

106. Documented progression of locally advanced unresectable or metastatic breast cancer, or documented progression within 6 months after completing adjuvant therapy;

107. Baseline measurable disease according to RECIST 1.1 from non-irradiated region or progressed within a previous radiation field;

108. Left ventricular ejection fraction (LVEF)≥50% at baseline (within 42 days of first trial treatment) as determined by either ECHO (preferred) or MUGA;

109. ECOG status 0 or 1;

110. Adequate organ function assessed within 7 days prior to first trial treatment:
Hematological function
ANC≥1.5×10⁹/L;
Hemoglobin≥9 g/dL;
Platelets≥100×10⁹/L;
Renal function
Calculated creatinine clearance≥60 mL/min (Cockcroft-Gault method);
Hepatic function
Total bilirubin≤1.5×ULN (or 2.5×ULN for documented Gilberts' syndrome);
ALT/AST≤3.0×ULN (or 5.0×ULN for documented liver metastasis);
INR or aPTT≤1.5×ULN;

111. Have a life expectancy of at least 3 months;

112. If a male subject with a partner with childbearing potential, be willing to use a highly effective method of contraception (with a failure rate of less than 1.0% per year) from first study treatment to 24 weeks after completion of the trial treatment.

### Exclusion Criteria

E01. Untreated active CNS metastasis or leptomeningeal metastasis. Subjects may be eligible provided they are treated and clinically stable for at least 4 weeks and have no evidence of new or enlarging brain metastases and also are off steroids 7 days for treating brain metastasis prior to first trial treatment;

E02. Uncontrolled tumor-related pain. Patients requiring narcotic pain medication must be on a stable regimen before first trial treatment for at least 2 weeks;

E03. Has received other anti-tumor treatment, including traditional Chinese medicine which has approved anti-tumor indication within 4 weeks prior to the first trial treatment;

E04. Major surgery for any reason, except diagnostic biopsy, within 4 weeks of the first administration of trial treatment and/or if the subject has not fully recovered from the surgery within 4 weeks of the first administration of trial treatment;

E05. Curative radiation within 3 months of the first dose of trial treatment. Radiation to more than 30% of the bone marrow or with a wide field of radiation should not be used within 4 weeks prior to the first administration of trial treatment. Symptomatic lesions (eg, bone metastases or metastases causing nerve impingement) or asymptomatic metastatic lesions (eg, likely cause functional deficits or intractable pain if further growth) amenable to palliative radiotherapy should be treated prior to first trial treatment and patients should be recovered from the effects of radiation;

E06. Uncontrolled hypercalcemia (>1.5 mmol/L ionized calcium or calcium >12 mg/dL or corrected serum calcium >ULN) or symptomatic hypercalcemia requiring continued use of bisphosphonate therapy;

E07. Is currently participating and receiving an investigational drug or has participated in a study of an investigational drug within 4 weeks or within 5 half-lives of the investigational drug prior to the first dose of trial treatment, whichever is shorter;

E08. Prior treatment with any CDK inhibitor, or with everolimus, or any agent whose mechanism of action is to inhibit the PI3K-mTOR pathway;

E09. Current use of food or drugs known to be potent CYP3A4 inhibitors, drugs known to be potent CYP3A4 inducers, and drugs that are known to prolong the QT interval;

E10. History of exposure to the following cumulative doses of anthracyclines
Doxorubicin or liposomal doxorubicin >360 mg/m²;
Epirubicin >720 mg/m²;
Mitoxantrone >120 mg/m² and idarubicin >90 mg/m²;
Other anthracycline >equivalent of 360 mg/m² of doxorubicin. If more than 1 anthracyclines have been used, the cumulative dose must not exceed the equivalent of 360 mg/m² of doxorubicin;

E11. Previous malignant disease other than the target malignancy to be investigated in this study with the exception of adequately treated non-melanomatous cancers of the skin, in situ carcinoma of the prostate/cervical cancer, superficial bladder cancer or other malignancy treated at least 5 years previously with surgery and/or curative radiotherapy, and there is no evidence of recurrence since that time;

E12. History of uncontrolled intercurrent illness including but not limited to:
Active HBV or HCV infection;
Known HIV infection or known history of acquired immune deficient syndrome (AIDS);
Active tuberculosis infection;
Active infection within 2 weeks prior to first trial treatment that requires the use of systemic antibiotics;
Has interstitial lung disease, or a history of pneumonitis that required oral or intravenous glucocorticoids to assist with management;
Has active cardiovascular disease or a history of cardiovascular dysfunction including any of the following:
History of angina pectoris, symptomatic pericarditis, myocardial infarction, coronary/peripheral artery bypass graft, cerebrovascular accident including transient ischemic attack, or symptomatic pulmonary embolism within 6 months prior to first trial treatment;
History of documented congestive heart failure (New York Heart Association function classification III-IV); documented cardiomyopathy;
History of LVEF decline to below 50% or absolute decrease for more than 15% during or after prior HER2-targeted therapy (neo/adjuvant, locally advanced or metastatic disease);
Serious cardiac arrhythmia requiring medication (including corrected QT interval prolongation of >470 msec <based on the mean value of the triplicate ECGs>calculated according to Fridericia, pacemaker, prior diagnosis of QT prolongation, Brugada syndrome or Torsade de Pointes; other ongoing cardiac dysrhythmias of CTCAE Grade ≥2; ongoing atrial fibrillation of any grade;
Hypertension uncontrolled by standard therapies (not stabilized to 150/90 mmHg);
Impairment of gastro-intestinal (GI) function or GI disease that may significantly alter the absorption of CDK inhibitor, such as history of GI surgery with may result in intestinal blind loops and patients with clinically significant gastroparesis, short bowl syndrome, unresolved nausea, vomiting, active inflammatory bowel disease or diarrhea of CTCAE Grade ≥1;
Prior hematopeietic stem cell transplant or solid organ transplant;

E13. Persisting toxicity related to prior therapy (including any prior investigational therapy) of CTCAE≥grade 2 or related toxicity not recovery to baseline, with the exception of alopecia of any grade;

E14. Known severe hypersensitivity reactions to antibody drug (CTCAE Grade ≥3), any history of anaphylaxis, uncontrolled asthma (that is, 3 or more features of partially controlled asthma), or any history of severe drug hypersensitivity (for example immune mediated liver toxicity, immune mediated thrombocytopenia or anemia);

E15. Other medical conditions that at the discretion of investigator interfere with the requirements of the trial in terms of safety or efficacy evaluation, or treatment compliance. These include but are not limited to psychiatric or substance abuse disorder, moderate to large pleural fluid/cardiac effusion/ascites, or recurrent/refractory pleural fluid/cardiac effusion/ascites.

### Statistical Methods

This study is planned to enroll 30 HR⁻/HER2-positive and 30 HR⁺/HER2-positive MBC subjects. Based on a sample size of 30, the table below shows the number of responders, estimated ORR and its associated 95% confidence intervals using the Clopper Pearson method.

| Number of subjects | Responders (%) | 95% confidence interval |
|---|---|---|
| 30 | 6 (20%) | 8%, 39% |
| | 8 (27%) | 12%, 46% |
| | 10 (33%) | 17%, 53% |
| | 12 (40%) | 23%, 59% |
| | 14 (47%) | 28%, 66% |
| | 16 (53%) | 34%, 72% |
| | 18 (60%) | 41%, 77% |

### Example 3 Assessment

According to the study in example 2, the results in the clinical trail will be recorded in the following table.

| | Assessment | | | | | | | | | | | |
|---|---|---|---|---|---|---|---|---|---|---|---|---|
| | | | | | | | | | End of | 30-day | 84-day | |
| | | | | | | | | | treatment | Safety | Safety | Survival |
| | Screening | Treatment Period | | | | | | | (EOT) | Follow-up | Follow-up | Follow-up |
| | Visit | | | | | | | | | | | |
| | | | | | | | | | | 30-day | 84-day | Every 12 |
| | Screening | C1D1 | C1D8 | C1D15 | C2D1 | C2D15 | CnD1 | CnD15 | EOT | FU | FU | weeks |
| | Day (d) | | | | | | | | | | | |
| | | | | | | | | | (within | 30 days | 84 days | Every 12 |
| | | | | | | | | | 7 days | after | after | weeks after |
| | | | | | | | | | after | last dose | last dose | last dose |
| | | | | | | | | | last dose | of trial | of trial | of trial |
| | | 1 | 8 | 15 | 1 | 15 | 1 | 15 | of trial | treatment | treatment | treatment |
| | (−21~−1) | (±3 d) | (±1 d) | (±3 d) | (±3 d) | (±3 d) | (±3 d) | (±3 d) | treatment) | (±7 d) | (±14 d) | (±14 d) |
| General | | | | | | | | | | | | |
| procedure | | | | | | | | | | | | |
| Informed | X | | | | | | | | | | | |
| consent ¹ | | | | | | | | | | | | |
| Inclusion/ | X | | | | | | | | | | | |
| exclusion | | | | | | | | | | | | |
| criteria | | | | | | | | | | | | |
| Demography | X | | | | | | | | | | | |
| and history of | | | | | | | | | | | | |
| alcohol and | | | | | | | | | | | | |
| tobacco use ² | | | | | | | | | | | | |
| Medical history ³ | X | | | | | | | | | | | |
| Oncology | X | | | | | | | | | | | |
| history ⁴ | | | | | | | | | | | | |
| Prior and | X | X | | X | X | X | X | X | X | X | | |
| concomitant | | | | | | | | | | | | |
| medications and | | | | | | | | | | | | |
| procedures ⁵ | | | | | | | | | | | | |
| New anti-cancer | | | | | | | | | X | X | X | X |
| therapy | | | | | | | | | | | | |
| Survival status | | | | | | | | | | | | X |
| Clinical examination | | | | | | | | | | | | |
| and test | | | | | | | | | | | | |
| Adverse event ⁶ | X | X | | | | | | | X | X | (X) | (X) |
| Full physical | X | | | | | | | | X | | | |
| examination ⁷ | | | | | | | | | | | | |
| Symptom- | | X | X | X | X | X | X | X | | X | X | |
| directed | | | | | | | | | | | | |
| examination ²⁸ | | | | | | | | | | | | |
| Height (in cm) | X | | | | | | | | | | | |
| Body weight (to | X | X | | | X | X | X | | X | X | X | |
| the nearest 0.1 | | | | | | | | | | | | |
| kilogram [kg]) ²⁷ | | | | | | | | | | | | |
| Vital signs ^{8, 27} | X | X | | | X | | X | | X | X | X | |
| 12-lead ECG ⁹ | X | | | | | | | | X | | | |
| ECOG | X | X | | | X | | X | | X | X | X | |
| performance | | | | | | | | | | | | |
| status ²⁷ | | | | | | | | | | | | |
| Local laboratory | | | | | | | | | | | | |
| test/evaluation | | | | | | | | | | | | |
| Hematology | X ²⁸ | | | | X | | X | | X | (X) ²⁹ | | |
| test ^{10, 27} | | | | | | | | | | | | |
| Coagulation test | X ²⁸ | | | | | | | | | | | |
| (aPTT, PT, INR) | | | | | | | | | | | | |
| Serum | X ²⁸ | | | | X | | X | | X | (X) ²⁹ | | |
| chemistry ^{11, 27} | | | | | | | | | | | | |
| Urinalysis ¹² | X | X | | | X | | X | | X | (X) ²⁹ | | |
| HBV, HCV, | X | | | | | | | | | | | |
| HIV ¹³ | | | | | | | | | | | | |
| TB test ¹⁴ | (X) | | | | | | | | | | | |
| LVEF ¹⁵ | X | Every 12 weeks | | | | | | | X | | X | X |
| Central laboratory | | | | | | | | | | | | |
| test/evaluation | | | | | | | | | | | | |
| Central | X | | | | | | | | | | | |
| confirmation of | | | | | | | | | | | | |
| HER2 status ¹⁶ | | | | | | | | | | | | |
| Biomarker | X | | | | | | | | | | | |
| (tumor tissue | | | | | | | | | | | | |
| collection) | | | | | | | | | | | | |
| (optional) ¹⁷ | | | | | | | | | | | | |
| Biomarker | X | | | | | | | | (X), upon | | | |
| (blood) | | | | | | | | | disease | | | |
| (optional) ¹⁸ | | | | | | | | | progres- | | | |
| | | | | | | | | | sion | | | |
| Pharmacokinetics | | X | X | X | X | | (X) | (X) | X | X | X | |
| (HER2 | | | | | | | | | | | | |
| INHIBITOR) ¹⁹ | | | | | | | | | | | | |
| ADA (HER2 | | X | | | X | | (X) | (X) | X | X | X | |
| INHIBITOR) ²⁰ | | | | | | | | | | | | |
| Tumor evaluation | | | | | | | | | | | | |
| Tumor imaging | X | Every 8 weeks within 12 months and every 12 | | | | | | | (X) | (X) | (X) | (X) |
| (chest/abdomen/ | | weeks after 12 months (±7 days) | | | | | | | | | | |
| pelvis) ²¹ | | | | | | | | | | | | |
| Tumor imaging | X | (clinically indicated) | | | | | | | | | | |
| (brain if | | | | | | | | | | | | |
| applicable) ²² | | | | | | | | | | | | |
| Bone scan (if | (X) | (clinically indicated) | | | | | | | | | | |
| applicable) ²³ | | | | | | | | | | | | |
| Photographs of | (X) | (clinically indicated) | | | | | | | | | | |
| all superficial | | | | | | | | | | | | |
| lesions (if | | | | | | | | | | | | |
| applicable) ²⁴ | | | | | | | | | | | | |
| Investigational | | | | | | | | | | | | |
| drug | | | | | | | | | | | | |
| HER2 inhibitor | | X | X | | X | | X | X | | | | |
| administration ²⁵ | | | | | | | | | | | | |
| CDK inhibitor ²⁶ | | X | | X | X | X | X | X | | | | |
| Fulvestrant | | X | | X | X | | X | | | | | |
| (HR⁺/HER2− | | | | | | | | | | | | |
| positive MBC | | | | | | | | | | | | |
| only) | | | | | | | | | | | | |

¹ Informed consent should be obtained before any trial related procedure and treatment;
² Demographic data include date of birth, sex, race and ethnicity;
³ Medical history includes past and concurrent nonmalignant diseases and treatment, past and concurrent malignant diseases and treatment;
⁴ Oncology history includes detailed history of the target indication of this study including histopathological diagnosis, grading and staging in accordance with AJCC version 8 at initial diagnosis and at the time of diagnosing locally advanced unresectable or metastatic disease; all therapy used for prior treatment of the tumor (including surgery, radiotherapy, chemotherapy, and immunotherapy); any other conditions that were treated with chemotherapy, radiation therapy, or immunotherapy; current cancer signs and symptoms, and AEs effects from current and/or previous anticancer treatments; and current cancer disease status;
⁵ All medications (including herbal medications) taken and procedures performed within 28 days prior to first trial treatment should be recorded; radiation therapy other than the target indication of this study performed within 3 months prior to first trial treatment should be recorded;
⁶ All AEs will be documented from signed inform consent until the 30-day safety follow up visit. After this visit, related SAE (cardiac or non-cardiac) MUST be collected and reported regardless of the time elapsed from the last HER2 inhibitor drug administration. All cardiac AEs occurring during the study and up to 12 months after last HER2 inhibitor drug administration must be reported irrespective of causal relationship (related or unrelated) or severity (serious or non-serious);
⁷ Full physical examination includes an examination of general appearance, skin, neck (including thyroid), eyes, ears, nose, throat, lungs, heart, abdomen, back, lymph nodes, extremities, and a basic nervous system evaluation;
⁸ Vital signs include temperature, respiration rate, pulse rate and blood pressure;
⁹ 12-lead ECG will be performed in the supine position after the subject has been breathing quietly for 5 minutes; The ECG results will be used to evaluate heart rate, atrial-ventricular conduction, QR and QT intervals, and possible arrhythmias. Interpretation of the ECG trace must be made by a qualified physician and documented on the ECG eCRF. Each ECG trace should be labeled with the Study Number, Subject Identifier Number, and date, and kept in the source documents at the site;
¹⁰ Hematology test includes absolute lymphocyte count, absolute neutrophil count, hematocrit, hemoglobin, platelet count, red blood cells, white blood cells and differential count, red blood cell morphology, reticulocytes, mean corpuscular hemoglobin, mean corpuscular volume, mean corpuscular hemoglobin concentration;
¹¹ Serum chemistry test includes albumin, alkaline phosphatase, alanine aminotransferase, amylase, aspartate aminotransferase, gamma-glutamyl transferase, blood urea nitrogen/total urea, calcium, chloride, creatine kinase, creatinine, glucose, lactate dehydrogenase, lipase, phosphorus/phosphates, magnesium, potassium, sodium, troponin, total bilimbin, total protein;
¹² Urinalysis includes bilirubin, blood, glucose, ketones, pH, protein, specific gravity, and color and appearance. If urinalysis is positive for protein, segments and 24-hour urine protein exam should be performed;
¹³ HBV, HCV and HIV tests include HBsAb, HCV Ab and anti-HIV1/2. If HBsAb is positive, HBsAg, HBeAb, HBeAg, HBcAb and HBV DNA should be measured to exclude active HBV infection as clinically indicated; if HCV Ab is positive, HCV RNA should be measured to exclude active HCV infection as clinically indicated;
¹⁴ For subject who has history of untreated or inadequately treated latent or active TB infection, recent contacts of persons with infectious tuberculosis, nor clinical signs and symptoms indicative of tuberculosis infection, a negative QuantiFERON ®-TB Gold In-Tube test (TB Quantiferon test) or if unavailable or indeterminate upon retest, a Mantoux Purified Derivative skin test result of <5 mm of induration performed according to local standard within 3 months prior to screening is acceptable to declare no suspected latent nor active tuberculosis infection;
For subject who has history of either untreated or inadequately treated latent or active TB infection, recent contacts of persons with infectious tuberculosis, or with signs and symptoms that indicate TB infection, below measures are recommended to exclude suspected latent tuberculosis infection:
A negative Mantoux Purified Derivative skin test result of <5 mm of induration performed according to local standard within 3 months prior to screening (note: subject who received BCG vaccination should be tested with QuantiFERON ®-TB Gold In-Tube test);
A chest X-ray taken at the screening visit or within 3 months prior to the screening visit does not suggest a latent nor active TB infection;
For subject who has previously received adequate course of therapy for either latent (9 months of isoniazid in areas where rate of multi-drug resistant TB infection below 5% or other acceptable regimen) or active (with an acceptable regimen) TB infection, a chest X-ray must be obtained at the screening visit or within 3 months prior to the screening visit to exclude latent or active TB infection. Under these circumstances, neither PPD nor a QuantiFERON ®-TB Gold In-Tube test need to be obtained;
¹⁵ The baseline LVEF assessment should be performed as close as possible to the first trial treatment, but at a maximum of 42 days prior to first trial treatment. During treatment period, LVEF will be assessed every 12 weeks. May perform more frequent LVEF assessment as needed for cardiac safety. If an LVEF assessment must be performed earlier or later, subsequent assessment should be conducted according to the original schedule from the date of first trial treatment. Patients for whom study treatment was permanently discontinued due to a drop in LVEF should continue to monitor LVEF as clinically indicated until LVEF values return to ≥50% or 1 year, whichever occurs first;
¹⁶ For the purpose of eligibility, documentation of HR and HER2 status from tumor tissue collected at the time of diagnosing locally advanced unresectable or metastatic disease will be based on local laboratory results utilizing an institutional standard assay. Meanwhile, tumor tissue collected at the time of diagnosing locally advanced unresectable or metastatic disease is required to be submitted to Sponsor designated central laboratory to confirm HER2 status based on IHC and FISH methods. Archived formalin-fixed paraffin embedded (FFPE) specimen will be collected. If archived metastatic or locally advanced unresectable tumor FFPE specimen is not available, a de novo biopsy will be required. Minimum of 5 slides are required;
¹⁷ A tumor biopsy should be collected at Screening unless tissue (blocks or slides) from an archival specimen (biopsy or surgery) is available and was obtained no more than 2 years prior to Screening. Core needle biopsies, excisional biopsies, punch biopsies, and surgical specimens are suitable. Fine needle aspiration biopsies are not suitable. Biopsies are only to be obtained from safely accessible tumor tissue/sites. Priority 1: tumor-containing FFPE tissue block; Priority 2: if the tumor-containing FFPE tissue block cannot be provided in total, sections from this block should be provided that are freshly cut (within 1 week), 4 μm thick, and mounted on SuperFrost Plus microscope slides. Subjects should be encouraged to provide as many slides as possible, and preferably, approximately 5~10 slides should be provided in total;
¹⁸ Approximately 10 mL peripheral blood samples at Screening and at the time of disease progression is to be collected for the measurement of ERBB2 amplification and tumor single cell sequencing to develop HER2 CDx and to explore drug resistant mechanism;
¹⁹ During treatment period, HER2 inhibitor pharmacokinetics samples will be collected at pre-dose of day 1 of cycles 1, 2, 3, 5, and every other cycle within first 12 months and every 4 cycles after 12 months (−60 min before HER2 inhibitor dosing); at pre-dose of cycle 1 days 8 and 15; at end of infusion of days 1 and 8 of cycle 1, and day 1 of cycles 3 and 5 (within 30 min after completion of HER2 inhibitor infusion); at 30- and 84-day safety follow up;
²⁰ During treatment period, HER2 inhibitor ADA samples will be collected at pre-dose of cycles 1, 2, 3, 5, and every other cycle within first 12 months and every 4 cycles after 12 months (−60 min before KN046 dosing) along with PK sample collection; at 30- and 84-day safety follow up;
²¹ Tumor assessment will be performed using CT/MRI assessments of chest, abdomen and pelvis. If MRI is used, CT of chest is mandatory. Screening tumor assessment should be performed within 21 days of first trial treatment in order to document baseline status of tumor disease using RECIST 1.1 target and non-target lesions. During treatment period, the tumor assessment visit time window is ±7 days. CT or MRI scan (if MRI is used, CT of chest is mandatory) should always be used the same to screening period. If a tumor response is documented during the study, confirmation of the response should be performed according to RECIST 1.1, preferably at 4~6 weeks apart, but no sooner than 4 weeks and no later than 8 weeks after the initial documentation of CR or PR. Confirmation of PR/CR can be done at an assessment later than the next assessment after the initial documentation of PR/CR. Tumor assessment will be performed every 8 weeks within 12 months and every 12 weeks thereafter until disease progression per RECIST 1.1, start of new anti-cancer therapy, death, or withdrawal of informed consent, whichever comes first. If treatment discontinuation is due to reasons other than RECIST 1.1 defined progressive disease, tumor assessment should continue until RECIST 1.1 defined progressive disease, start of new anti-cancer therapy, death, or withdrawal of informed consent, whichever occurrs first;
²² Brain CT/MRI scan (either, with contrast preferred) is required at screening if not performed within the previous 42 days prior to first trial treatment. During treatment period, brain CT/MRI scan should be done if clinically indicated by development of new specific symptoms;
²³ A bone scan should be performed if not be done within 3 months prior to first trial treatment. During treatment period, Bone scan should be done as clinically indicated. Any suspicious abnormalities identified on the bone scans at baseline or on subsequent bone scans MUST be confirmed by X-ray, CT scan with bone windows or MRI. The same modality must be used throughout the trial for confirmation for a given lesion and patient. Bone lesions identified at baseline will be followed up according to the same tumor assessment schedule;
²⁴ Clinical assessment of superficial disease must be carried out on the same date as the imaging studies and will include photographs of all superficial metastasis lesions. All lesions must be recorded in the eCRF;
²⁵ First HER2 inhibitor dose will be administered intravenously over 90 minutes (±15 minutes). If no infusion related reaction occurs after first dose, subsequent doses can be administered intravenously over 60 minutes (±15 minutes);
²⁶ CDK inhibitor will be administered at 125 mg/day orally for 3 weeks followed by 1 week off. Patients are required to complete patient diary on Day 1 of each cycle for drug accountability and return all bottles of CDK inhibitor;
²⁷ Examination or test results should be obtained before study drug administration;
²⁸ Examination or test results should be obtained within 7 days prior to first trial treatment;
²⁹ Only be performed when clinically significant abnormal findings are found at EOT visit;

### Example 4

The HER2 inhibitor of the present application and the CDK inhibitor Ribociclib were used for treating breast cancer in MCF-7 XENO model. Four groups were divided equally (n=6):

Group 1: Vehicle, PO, QD;

Group 2: HER2 inhibitor, 10 mg/kg (DO), 5 mg/Kg (D3), 2.5 mg/Kg (D10-), IP, QW;

Group 3: Ribociclib, 40 mg/Kg (DO-D6), 20 mg/Kg (D7-D13), 10 mg/Kg (D14-), PO, QD;

Group 4: HER2 inhibitor, 10 mg/kg (DO), 5 mg/Kg (D3), 2.5 mg/Kg (D10-), TP, QW; Ribociclib 40 mg/Kg (DO-D6), 20 mg/Kg (D7-D13), 10 mg/Kg (D14-), PO, QD.

The tumor volume in MCF-7 XENO model is illustrated in FIG. 4. It can be seen that the combination of the HER2 inhibitor and the CDK inhibitor Ribociclib can reduce the tumor volume significantly.

### Example 5 Study Design

This is an open-label, multicenter, dose-escalation and parallel-group expansion Phase 1b/2 clinical trial to evaluate the efficacy, safety and tolerability of HER2 inhibitor in combination with palbociclib with or without fulvestrant in women or male with HER2-positive metastatic breast cancer whose disease has progressed after treatment with trastuzumab and a taxane, at the minimum in any breast cancer disease setting. Patients could have received one prior hormonal treatment for MBC.

Eligible patients must have histologically or cytologically proven diagnosis of HER2-positive adenocarcinoma of the breast at the time of diagnosing locally advanced unresectable or metastatic disease. HER2 status will be confirmed in a central laboratory with use of archival paraffin-embedded tumor tissue. Tumor HER2 status will be considered positive if the central laboratory reports Grade 3+ staining intensity (on a scale of 0 to 3+) by means of IHC analysis or gene amplification by FISH.

Dose escalation will be performed at the following 2 dose levels:
Dose level 1
Patients with HR⁻/HER2-positive MBC will receive:
HER2 inhibitor 20 mg/kg IV Q2W (with 20 mg/kg loading on Days 1 and 8 of Cycle 1)+palbociclib 100 mg/day orally for 3 weeks followed by 1 week off,
Patients with HR⁺/HER2-positive MBC will receive:
HER2 inhibitor 20 mg/kg IV Q2W (with 20 mg/kg loading on Days 1 and 8 of Cycle 1)+palbociclib 100 mg/day orally for 3 weeks followed by 1 week off+fulvestrant 500 mg intramuscularly on Days 1 and 15 of Cycle 1, and then on Day 1 of each subsequent 28-day cycle;
Dose level 2
Patients with HR⁻/HER2-positive MBC will receive:
HER2 inhibitor 20 mg/kg IV Q2W (with 20 mg/kg loading on Days 1 and 8 of Cycle 1)+palbociclib 125 mg/day orally for 3 weeks followed by 1 week off,
Patients with HR⁺/HER2-positive MBC will receive:
HER2 inhibitor 20 mg/kg IV Q2W (with 20 mg/kg loading on Days 1 and 8 of Cycle 1)+palbociclib 125 mg/day orally for 3 weeks followed by 1 week off+fulvestrant 500 mg intramuscularly on Days 1 and 15 of Cycle 1, and then on Day 1 of each subsequent 28-day cycle;

The dose escalation phase uses a mTPI-2 (modified toxicity probability interval) design. 3˜6 subjects will be enrolled at each treatment cohort. The decision to escalate to the next dose level will be based on safety assessments after all subjects of a treatment cohort have reached Day 28 (dose limiting toxicity observation period). A scientific monitoring committee (SMC) will be responsible for decisions on dose escalation, determination of combination maximum tolerated dose (MTD) or recommended phase 2 dose (RP2D) and will be established before first patient dose. SMC may decide to explore higher, lower or interim dose levels based on available safety and efficacy data from HER2 inhibitor program. Once the combination MTD or RP2D has been established, the study will enter the parallel-group expansion phase.

In parallel-group expansion phase, 30 HR⁻/HER2-positive and 30 HR⁺/HER2-positive MBC patients will be enrolled and treated by combination RP2Ds. Patients with HR⁻/HER2-positive MBC will receive HER2 inhibitor in combination with palbociclib and patients with HR⁺/HER2-positive MBC will receive triple combination of HER2 INHIBITOR, palbociclib and fulvestrant.

After completion of the initial parallel-group expansion, further expansion in HR⁻/HER2-positive, HR⁺/HER2-positive or further expansion in HER2-intermediate or low expressing (defined as HER2 IHC1+ or 2+ AND FISH-negative) MBC patients may be added. Protocol amendment will be issued for further expansion after the approval from applicable committees.

Patients must have at least one measurable disease at baseline from non-irradiated region or progressed within a previous radiation field. Patients will undergo tumor assessment every 8 weeks for the first 12 months and then every 12 weeks, calculated from the date of first trial treatment. Tumor assessment will continue until progressive disease according to RECIST 1.1, initiation of new anti-cancer therapy, death, or withdrawal of consent, whichever occurs first.

Patients will continue to receive assigned trial treatment until progressive disease according to RECIST 1.1, clinical deterioration, unacceptable toxicity, initiation of new anti-cancer therapy, death, or withdrawal of consent, whichever comes first. Should palbociclib, HER2 inhibitor or fulvestrant related toxicity mandate discontinuation of perspective trial treatment, patients can continue to receive other study drugs.

Each subject enrolled will have a screening period (−21 to −1 day prior to first trial treatment), a treatment period, an end-of-treatment (EOT) visit, a 30-day safety follow-up visit, a 84-day safety follow-up visit and an overall survival follow-up. The study will end at 12 months after all subjects complete the last dose of trial treatment.

### Example 6 Introduction of STUDY

### Study Object

Primary Objective

Dose Escalation Phase
To determine MTD or RP2D of:
HER2 inhibitor in combination with palbociclib in HR⁻/HER2-positive MBC; and
HER2 inhibitor in combination with palbociclib and fulvestrant in HR⁺/HER2-positive MBC;

Parallel-Group Expansion Phase
To evaluate anti-tumor activity of HER2 inhibitor in combination with palbociclib in HR⁻/HER2-positive MBC;
To evaluate anti-tumor activity of HER2 inhibitor in combination with palbociclib and fulvestrant in HR⁺/HER2-positive MBC;

Secondary Objectives
To assess safety and tolerability of HER2 inhibitor in combination with palbociclib with or without fulvestrant;
To characterize pharmacokinetics of HER2 INHIBITOR;
To evaluate immunogenicity of HER2 INHIBITOR;

Exploratory Objectives
To explore relationship between HER2 inhibitor drug exposure and efficacy/safety findings;
To explore alterations in genes, proteins, and ribonucleic acids (RNAs) relevant to the cell cycle, drug targets, tumor sensitivity and/or resistance.

### Study Endpoints

Primary Endpoint

Dose Escalation Phase
Dose limiting toxicity (DLT);

Parallel-Group Expansion Phase
Objective response rate as assessed by the investigator according to RECIST 1.1;

Secondary Endpoints
Duration of response (DOR) and time to response (TTR);
6- and 12-months PFS and OS rates;
Clinical benefit rate (CBR), defined as proportion of complete response (CR), partial response (PR) or stable disease (SD)≥24 weeks; disease control rate (DCR), defined as proportion of CR, PR or SD;
Incidence and severity (as graded by CTCAE v5.0), seriousness and relationship to the trial treatments, abnormal findings on any laboratory test and physical examination. All cardiac AEs occurring during the study and up to 12 months after last HER2 inhibitor drug administration must be reported irrespective of causal relationship (related or unrelated) or severity (serious or non-serious);
PK parameters of HER2 inhibitor derived from population PK analysis, including but not limited to AUC, Cₘₐₓ, Cₘᵢₙ, CLand T_{1/2};
Status (positive or negative) and serum titers of anti-HER2 inhibitor antibody and neutralizing capacity;

Exploratory Endpoints
Tumor tissue biomarkers, including genes (eg, HER2 mutation, copy number of HER2, CCND1 and CDKN2A, PIK3CA mutation), proteins (eg, Ki67, pRb, CCNE1) and RNA expression (eg, cdk4, cdk6).

### Study Population

### Inclusion Criteria

101. Signed informed consent;

102. Postmenopausal female or male subjects 18 years of age or older. Postmenopausal women are defined by at least one of the following criteria:
Age ≥60 years;
Age <60 years and cessation of regular menses for at least 12 consecutive months with no alternative pathological or physiological cause; and serum estradiol and FSH level within the laboratory's reference range for postmenopausal females;
Documented bilateral oophorectomy;
Medically confirmed ovarian failure;

103. Histologically or cytologically proven diagnosis of breast cancer with evidence of metastatic or locally advanced disease not amenable to resection or radiation therapy with curative intent;

104. HER2-positive tumor based on tumor tissue collected at the time of diagnosing locally advanced unresectable or metastatic disease. HER2-positivity is defined as IHC 3+ and/or HER2 gene-amplified by FISH. HER2 status determined by local laboratory can be used to determine eligibility for this study however tissue samples MUST be submitted to a Sponsor-designated central laboratory to confirm HER2 status;

105. Prior treatment for breast cancer in the adjuvant, locally advanced unresectable or metastatic setting must include a taxane and trastuzumab;

106. Documented progression of locally advanced unresectable or metastatic breast cancer, or documented progression within 6 months after completing adjuvant therapy;

107. Baseline measurable disease according to RECIST 1.1 from non-irradiated region or progressed within a previous radiation field;

108. Left ventricular ejection fraction (LVEF)≥50% at baseline (within 42 days of first trial treatment) as determined by either ECHO (preferred) or MUGA;

109. ECOG status 0 or 1;

110. Adequate organ function assessed within 7 days prior to first trial treatment:
Hematological function
ANC≥1.5×10⁹/L;
Hemoglobin≥9 g/dL;
Platelets≥100×10⁹/L;
Renal function
Calculated creatinine clearance≥60 mL/min (Cockcroft-Gault method);
Hepatic function
Total bilirubin≤1.5×ULN (or 2.5×ULN for documented Gilberts' syndrome);
ALT/AST≤3.0×ULN (or 5.0×ULN for documented liver metastasis);
INR or aPTT≤1.5×ULN;

111. Have a life expectancy of at least 3 months;

112. If a male subject with a partner with childbearing potential, be willing to use a highly effective method of contraception (with a failure rate of less than 1.0% per year) from first study treatment to 24 weeks after completion of the trial treatment.

### Exclusion Criteria

E01. Untreated active CNS metastasis or leptomeningeal metastasis. Subjects may be eligible provided they are treated and clinically stable for at least 4 weeks and have no evidence of new or enlarging brain metastases and also are off steroids 7 days for treating brain metastasis prior to first trial treatment;

E02. Uncontrolled tumor-related pain. Patients requiring narcotic pain medication must be on a stable regimen before first trial treatment for at least 2 weeks;

E03. Has received other anti-tumor treatment, including traditional Chinese medicine which has approved anti-tumor indication within 4 weeks prior to the first trial treatment;

E04. Major surgery for any reason, except diagnostic biopsy, within 4 weeks of the first administration of trial treatment and/or if the subject has not fully recovered from the surgery within 4 weeks of the first administration of trial treatment;

E05. Curative radiation within 3 months of the first dose of trial treatment. Radiation to more than 30% of the bone marrow or with a wide field of radiation should not be used within 4 weeks prior to the first administration of trial treatment. Symptomatic lesions (eg, bone metastases or metastases causing nerve impingement) or asymptomatic metastatic lesions (eg, likely cause functional deficits or intractable pain if further growth) amenable to palliative radiotherapy should be treated prior to first trial treatment and patients should be recovered from the effects of radiation;

E06. Uncontrolled hypercalcemia (>1.5 mmol/L ionized calcium or calcium >12 mg/dL or corrected serum calcium >ULN) or symptomatic hypercalcemia requiring continued use of bisphosphonate therapy;

E07. Is currently participating and receiving an investigational drug or has participated in a study of an investigational drug within 4 weeks or within 5 half-lives of the investigational drug prior to the first dose of trial treatment, whichever is shorter;

E08. Prior treatment with any CDK inhibitor, or with everolimus, or any agent whose mechanism of action is to inhibit the PI3K-mTOR pathway;

E09. Current use of food or drugs known to be potent CYP3A4 inhibitors, drugs known to be potent CYP3A4 inducers, and drugs that are known to prolong the QT interval;

E10. History of exposure to the following cumulative doses of anthracyclines
Doxorubicin or liposomal doxorubicin >360 mg/m²;
Epirubicin >720 mg/m²;
Mitoxantrone >120 mg/m² and idarubicin >90 mg/m²;
Other anthracycline >equivalent of 360 mg/m² of doxorubicin. If more than 1 anthracyclines have been used, the cumulative dose must not exceed the equivalent of 360 mg/m² of doxorubicin;

E11. Previous malignant disease other than the target malignancy to be investigated in this study with the exception of adequately treated non-melanomatous cancers of the skin, in situ carcinoma of the prostate/cervical cancer, superficial bladder cancer or other malignancy treated at least 5 years previously with surgery and/or curative radiotherapy, and there is no evidence of recurrence since that time;

E12. History of uncontrolled intercurrent illness including but not limited to:
Active HBV or HCV infection;
Known HIV infection or known history of acquired immune deficient syndrome (AIDS);
Active tuberculosis infection;
Active infection within 2 weeks prior to first trial treatment that requires the use of systemic antibiotics;
Has interstitial lung disease, or a history of pneumonitis that required oral or intravenous glucocorticoids to assist with management;
Has active cardiovascular disease or a history of cardiovascular dysfunction including any of the following:
History of angina pectoris, symptomatic pericarditis, myocardial infarction, coronary/peripheral artery bypass graft, cerebrovascular accident including transient ischemic attack, or symptomatic pulmonary embolism within 6 months prior to first trial treatment;
History of documented congestive heart failure (New York Heart Association function classification III-IV); documented cardiomyopathy;
History of LVEF decline to below 50% or absolute decrease for more than 15% during or after prior HER2-targeted therapy (neo/adjuvant, locally advanced or metastatic disease);
Serious cardiac arrhythmia requiring medication (including corrected QT interval prolongation of >470 msec <based on the mean value of the triplicate ECGs>calculated according to Fridericia, pacemaker, prior diagnosis of QT prolongation, Brugada syndrome or Torsade de Pointes; other ongoing cardiac dysrhythmias of CTCAE Grade ≥2; ongoing atrial fibrillation of any grade;
Hypertension uncontrolled by standard therapies (not stabilized to 150/90 mmHg);
Impairment of gastro-intestinal (GI) function or GI disease that may significantly alter the absorption of palbociclib, such as history of GI surgery with may result in intestinal blind loops and patients with clinically significant gastroparesis, short bowl syndrome, unresolved nausea, vomiting, active inflammatory bowel disease or diarrhea of CTCAE Grade ≥1;
Prior hematopeietic stem cell transplant or solid organ transplant;

E13. Persisting toxicity related to prior therapy (including any prior investigational therapy) of CTCAE≥grade 2 or related toxicity not recovery to baseline, with the exception of alopecia of any grade;

E14. Known severe hypersensitivity reactions to antibody drug (CTCAE Grade ≥3), any history of anaphylaxis, uncontrolled asthma (that is, 3 or more features of partially controlled asthma), or any history of severe drug hypersensitivity (for example immune mediated liver toxicity, immune mediated thrombocytopenia or anemia);

E15. Other medical conditions that at the discretion of investigator interfere with the requirements of the trial in terms of safety or efficacy evaluation, or treatment compliance. These include but are not limited to psychiatric or substance abuse disorder, moderate to large pleural fluid/cardiac effusion/ascites, or recurrent/refractory pleural fluid/cardiac effusion/ascites.

### Statistical Methods

This study is planned to enroll 30 HR⁻/HER2-positive and 30 HR⁺/HER2-positive MBC subjects. Based on a sample size of 30, the table below shows the number of responders, estimated ORR and its associated 95% confidence intervals using the Clopper Pearson method.

| Number of subjects | Responders (%) | 95% confidence interval |
|---|---|---|
| 30 | 6 (20%) | 8%, 39% |
| | 8 (27%) | 12%, 46% |
| | 10 (33%) | 17%, 53% |
| | 12 (40%) | 23%, 59% |
| | 14 (47%) | 28%, 66% |
| | 16 (53%) | 34%, 72% |
| | 18 (60%) | 41%, 77% |

### Example 7 Assessment

According to the study in example 6, the results in the clinical trail will be recorded in the following table:

| | Assessment | | | | | | | | | | | |
|---|---|---|---|---|---|---|---|---|---|---|---|---|
| | | | | | | | End of | | | 30-day | 84-day | |
| | | | | | | | treatment | | | Safety | Safety | Survival |
| | Screening | Treatment Period | | | | | (EOT) | | | Follow-up | Follow-up | Follow-up |
| | Visit | | | | | | | | | | | |
| | | | | | | | | | | 30-day | 84-day | Every ¹² |
| | Screening | C1D1 | C1D8 | C1D15 | C2D1 | C2D15 | CnD1 | CnD15 | EOT | FU | FU | weeks |
| | Day (d) | | | | | | | | | | | |
| | | | | | | | | | (within | 30 days | 84 days | Every 12 |
| | | | | | | | | | 7 days | after | after | week after |
| | | | | | | | | | after | last dose | last dose | last dose |
| | | | | | | | | | last dose | of trial | of trial | of trial |
| | | 1 | 8 | 15 | 1 | 15 | 1 | 15 | of trial | treatment | treatment | treatment |
| | (−21~−1) | (±3 d) | (±1 d) | (±3 d) | (±3 d) | (±3 d) | (±3 d) | (±3 d) | treatment) | (±7 d) | (±14 d) | (±14 d) |
| General | | | | | | | | | | | | |
| procedure | | | | | | | | | | | | |
| Informed | X | | | | | | | | | | | |
| consent ¹ | | | | | | | | | | | | |
| Inclusion/ | X | | | | | | | | | | | |
| exclusion | | | | | | | | | | | | |
| criteria | | | | | | | | | | | | |
| Demography | X | | | | | | | | | | | |
| and history of | | | | | | | | | | | | |
| alcohol and | | | | | | | | | | | | |
| tobacco use ² | | | | | | | | | | | | |
| Medical history ³ | X | | | | | | | | | | | |
| Oncology | X | | | | | | | | | | | |
| history ⁴ | | | | | | | | | | | | |
| Prior and | X | X | | X | X | X | X | X | X | X | | |
| concomitant | | | | | | | | | | | | |
| medications and | | | | | | | | | | | | |
| procedures ⁵ | | | | | | | | | | | | |
| New anti-cancer | | | | | | | | | X | X | X | X |
| therapy | | | | | | | | | | | | |
| Survival status | | | | | | | | | | | | X |
| Clinical examination | | | | | | | | | | | | |
| and test | | | | | | | | | | | | |
| Adverse event ⁶ | X | X | | | | | | | X | X | (X) | (X) |
| Full physical | X | | | | | | | | X | | | |
| examination ⁷ | | | | | | | | | | | | |
| Symptom- | | X | X | X | X | X | X | X | | X | X | |
| directed | | | | | | | | | | | | |
| examination ²⁸ | | | | | | | | | | | | |
| Height (in cm) | X | | | | | | | | | | | |
| Body weight (to | X | X | | | X | X | X | | X | X | X | |
| the nearest 0.1 | | | | | | | | | | | | |
| kilogram [kg]) ²⁷ | | | | | | | | | | | | |
| Vital signs ^{8, 27} | X | X | | | X | | X | | X | X | X | |
| 12-leadECG ⁹ | X | | | | | | | | X | | | |
| ECOG | X | X | | | X | | X | | X | X | X | |
| performance | | | | | | | | | | | | |
| status ²⁷ | | | | | | | | | | | | |
| Local laboratory | | | | | | | | | | | | |
| test/evaluation | | | | | | | | | | | | |
| Hematology | X ²⁸ | | | | X | | X | | X | (X) ²⁹ | | |
| test ^{10, 27} | | | | | | | | | | | | |
| Coagulation test | X ²⁸ | | | | | | | | | | | |
| (aPTT, PT, INR) | | | | | | | | | | | | |
| Serum | X ²⁸ | | | | X | | X | | X | (X) ²⁹ | | |
| chemistry ^{11, 27} | | | | | | | | | | | | |
| Urinalysis ¹² | X | X | | | X | | X | | X | (X) ²⁹ | | |
| HBV, HCV, | | | | | | | | | | | | |
| HIV ¹³ | | | | | | | | | | | | |
| TB test ¹⁴ | (X) | | | | | | | | | | | |
| LVEF ¹⁵ | X | Every 12 weeks | | | | | | | X | | X | X |
| Central laboratory | | | | | | | | | | | | |
| test/evaluation | | | | | | | | | | | | |
| Central | X | | | | | | | | | | | |
| confirmation of | | | | | | | | | | | | |
| HER2 status ¹⁶ | | | | | | | | | | | | |
| Biomarker | X | | | | | | | | | | | |
| (tumor tissue | | | | | | | | | | | | |
| collection) | | | | | | | | | | | | |
| (optional) ¹⁷ | | | | | | | | | | | | |
| Biomarker | X | | | | | | | | (X), | | | |
| (blood) | | | | | | | | | upon | | | |
| (optional) ¹⁸ | | | | | | | | | disease | | | |
| | | | | | | | | | progression | | | |
| Pharmacokinetics | | X | X | X | X | | (X) | (X) | X | X | X | |
| (HER2 | | | | | | | | | | | | |
| INHIBITOR) ¹⁹ | | | | | | | | | | | | |
| ADA (HER2 | | X | | | X | | (X) | (X) | X | X | X | |
| INHIBITOR) ²⁰ | | | | | | | | | | | | |
| Tumor evaluation | | | | | | | | | | | | |
| Tumor imaging | X | Every 8 weeks within 12 months and every 12 | | | | | | | (X) | (X) | (X) | (X) |
| (chest/abdomen/ | | weeks after 12 months (±7 days) | | | | | | | | | | |
| pelvis) ²¹ | | | | | | | | | | | | |
| Tumor imaging | X | (clinically indicated) | | | | | | | | | | |
| (brain if | | | | | | | | | | | | |
| applicable) ²² | | | | | | | | | | | | |
| Bone scan (if | (X) | (clinically indicated) | | | | | | | | | | |
| applicable) ²³ | | | | | | | | | | | | |
| Photographs of | (X) | (clinically indicated) | | | | | | | | | | |
| all superficial | | | | | | | | | | | | |
| lesions (if | | | | | | | | | | | | |
| applicable) ²⁴ | | | | | | | | | | | | |
| Investigational | | | | | | | | | | | | |
| drug | | | | | | | | | | | | |
| HER2 inhibitor | | X | X | | X | | X | X | | | | |
| administration ²⁵ | | | | | | | | | | | | |
| Palbociclib ²⁶ | | X | | X | X | X | X | X | | | | |
| Fulvestrant | | X | | X | X | | X | | | | | |
| (HR⁺/HER2− | | | | | | | | | | | | |
| positive MBC | | | | | | | | | | | | |
| only) | | | | | | | | | | | | |

¹ Informed consent should be obtained before any trial related procedure and treatment;
² Demographic data include date of birth, sex, race and ethnicity;
³ Medical history includes past and concurrent nonmalignant diseases and treatment, past and concurrent malignant diseases and treatment;
⁴ Oncology history includes detailed history of the target indication of this study including histopathological diagnosis, grading and staging in accordance with AJCC version 8 at initial diagnosis and at the time of diagnosing locally advanced unresectable or metastatic disease; all therapy used for prior treatment of the tumor (including surgery, radiotherapy, chemotherapy, and immunotherapy); any other conditions that were treated with chemotherapy, radiation therapy, or immunotherapy; current cancer signs and symptoms, and AEs effects from current and/or previous anticancer treatments; and current cancer disease status;
⁵ All medications (including herbal medications) taken and procedures performed within 28 days prior to first trial treatment should be recorded; radiation therapy other than the target indication of this study performed within 3 months prior to first trial treatment should be recorded;
⁶ All AEs will be documented from signed inform consent until the 30-day safety follow up visit. After this visit, related SAE (cardiac or non-cardiac) MUST be collected and reported regardless of the time elapsed from the last HER2 inhibitor drug administration. All cardiac AEs occurring during the study and up to 12 months after last HER2 inhibitor drug administration must be reported irrespective of causal relationship (related or unrelated) or severity (serious or non-serious);
⁷ Full physical examination includes an examination of general appearance, skin, neck (including thyroid), eyes, ears, nose, throat, lungs, heart, abdomen, back, lymph nodes, extremities, and a basic nervous system evaluation;
⁸ Vital signs include temperature, respiration rate, pulse rate and blood pressure;
⁹ 12-lead ECG will be performed in the supine position after the subject has been breathing quietly for 5 minutes; The ECG results will be used to evaluate heart rate, atrial-ventricular conduction, QR and QT intervals, and possible arrhythmias. Interpretation of the ECG trace must be made by a qualified physician and documented on the ECG eCRF. Each ECG trace should be labeled with the Study Number, Subject Identifier Number, and date, and kept in the source documents at the site;
¹⁰ Hematology test includes absolute lymphocyte count, absolute neutrophil count, hematocrit, hemoglobin, platelet count, red blood cells, white blood cells and differential count, red blood cell morphology, reticulocytes, mean corpuscular hemoglobin, mean corpuscular volume, mean corpuscular hemoglobin concentration;
¹¹ Serum chemistry test includes albumin, alkaline phosphatase, alanine aminotransferase, amylase, aspartate aminotransferase, gamma-glutamyl transferase, blood urea nitrogen/total urea, calcium, chloride, creatine kinase, creatinine, glucose, lactate dehydrogenase, lipase, phosphorus/phosphates, magnesium, potassium, sodium, troponin, total bilimbin, total protein;
¹² Urinalysis includes bilirubin, blood, glucose, ketones, pH, protein, specific gravity, and color and appearance. If urinalysis is positive for protein, segments and 24-hour urine protein exam should be performed;
¹³ HBV, HCV and HIV tests include HBsAb, HCV Ab and anti-HIV1/2. If HBsAb is positive, HBsAg, HBeAb, HBeAg, HBcAb and HBV DNA should be measured to exclude active HBV infection as clinically indicated; if HCV Ab is positive, HCV RNA should be measured to exclude active HCV infection as clinically indicated;
¹⁴ For subject who has history of untreated or inadequately treated latent or active TB infection, recent contacts of persons with infectious tuberculosis, nor clinical signs and symptoms indicative of tuberculosis infection, a negative QuantiFERON ®-TB Gold In-Tube test (TB Quantiferon test) or if unavailable or indeterminate upon retest, a Mantoux Purified Derivative skin test result of <5 mm of induration performed according to local standard within 3 months prior to screening is acceptable to declare no suspected latent nor active tuberculosis infection;
For subject who has history of either untreated or inadequately treated latent or active TB infection, recent contacts of persons with infectious tuberculosis, or with signs and symptoms that indicate TB infection, below measures are recommended to exclude suspected latent tuberculosis infection:
A negative Mantoux Purified Derivative skin test result of <5 mm of induration performed according to local standard within 3 months prior to screening (note: subject who received BCG vaccination should be tested with QuantiFERON ®-TB Gold In-Tube test);
A chest X-ray taken at the screening visit or within 3 months prior to the screening visit does not suggest a latent nor active TB infection;
For subject who has previously received adequate course of therapy for either latent (9 months of isoniazid in areas where rate of multi-drug resistant TB infection below 5% or other acceptable regimen) or active (with an acceptable regimen) TB infection, a chest X-ray must be obtained at the screening visit or within 3 months prior to the screening visit to exclude latent or active TB infection. Under these circumstances, neither PPD nor a QuantiFERON ®-TB Gold In-Tube test need to be obtained;
¹⁵ The baseline LVEF assessment should be performed as close as possible to the first trial treatment, but at a maximum of 42 days prior to first trial treatment. During treatment period, LVEF will be assessed every 12 weeks. May perform more frequent LVEF assessment as needed for cardiac safety. If an LVEF assessment must be performed earlier or later, subsequent assessment should be conducted according to the original schedule from the date of first trial treatment. Patients for whom study treatment was permanently discontinued due to a drop in LVEF should continue to monitor LVEF as clinically indicated until LVEF values return to ≥50% or 1 year, whichever occurs first;
¹⁶ For the purpose of eligibility, documentation of HR and HER2 status from tumor tissue collected at the time of diagnosing locally advanced unresectable or metastatic disease will be based on local laboratory results utilizing an institutional standard assay. Meanwhile, tumor tissue collected at the time of diagnosing locally advanced unresectable or metastatic disease is required to be submitted to Sponsor designated central laboratory to confirm HER2 status based on IHC and FISH methods. Archived formalin-fixed paraffin embedded (FFPE) specimen will be collected. If archived metastatic or locally advanced unresectable tumor FFPE specimen is not available, a de novo biopsy will be required. Minimum of 5 slides are required;
¹⁷ A tumor biopsy should be collected at Screening unless tissue (blocks or slides) from an archival specimen (biopsy or surgery) is available and was obtained no more than 2 years prior to Screening. Core needle biopsies, excisional biopsies, punch biopsies, and surgical specimens are suitable. Fine needle aspiration biopsies are not suitable. Biopsies are only to be obtained from safely accessible tumor tissue/sites. Priority 1: tumor-containing FFPE tissue block; Priority 2: if the tumor-containing FFPE tissue block cannot be provided in total, sections from this block should be provided that are freshly cut (within 1 week), 4 μm thick, and mounted on SuperFrost Plus microscope slides. Subjects should be encouraged to provide as many slides as possible, and preferably, approximately 5~10 slides should be provided in total;
¹⁸ Approximately 10 mL peripheral blood samples at Screening and at the time of disease progression is to be collected for the measurement of ERBB2 amplification and tumor single cell sequencing to develop HER2 CDx and to explore drug resistant mechanism;
¹⁹ During treatment period, HER2 inhibitor pharmacokinetics samples will be collected at pre-dose of day 1 of cycles 1, 2, 3, 5, and every other cycle within first 12 months and every 4 cycles after 12 months (−60 min before HER2 inhibitor dosing); at pre-dose of cycle 1 days 8 and 15; at end of infusion of days 1 and 8 of cycle 1, and day 1 of cycles 3 and 5 (within 30 min after completion of HER2 inhibitor infusion); at 30- and 84- day safety follow up;
²⁰ During treatment period, HER2 inhibitor ADA samples will be collected at pre-dose of cycles 1, 2, 3, 5, and every other cycle within first 12 months and every 4 cycles after 12 months (−60 min before KN046 dosing) along with PK sample collection; at 30- and 84-day safety follow up;
²¹ Tumor assessment will be performed using CT/MRI assessments of chest, abdomen and pelvis. If MRI is used, CT of chest is mandatory. Screening tumor assessment should be performed within 21 days of first trial treatment in order to document baseline status of tumor disease using RECIST 1.1 target and non-target lesions. During treatment period, the tumor assessment visit time window is ±7 days. CT or MRI scan (if MRI is used, CT of chest is mandatory) should always be used the same to screening period. If a tumor response is documented during the study, confirmation of the response should be performed according to RECIST 1.1, preferably at 4~6 weeks apart, but no sooner than 4 weeks and no later than 8 weeks after the initial documentation of CR or PR. Confirmation of PR/CR can be done at an assessment later than the next assessment after the initial documentation of PR/CR. Tumor assessment will be performed every 8 weeks within 12 months and every 12 weeks thereafter until disease progression per RECIST 1.1, start of new anti-cancer therapy, death, or withdrawal of informed consent, whichever comes first. If treatment discontinuation is due to reasons other than RECIST 1.1 defined progressive disease, tumor assessment should continue until RECIST 1.1 defined progressive disease, start of new anti-cancer therapy, death, or withdrawal of informed consent, whichever occurrs first;
²² Brain CT/MRI scan (either, with contrast preferred) is required at screening if not performed within the previous 42 days prior to first trial treatment. During treatment period, brain CT/MRI scan should be done if clinically indicated by development of new specific symptoms;
²³ A bone scan should be performed if not be done within 3 months prior to first trial treatment. During treatment period, Bone scan should be done as clinically indicated. Any suspicious abnormalities identified on the bone scans at baseline or on subsequent bone scans MUST be confirmed by X-ray, CT scan with bone windows or MRI. The same modality must be used throughout the trial for confirmation for a given lesion and patient. Bone lesions identified at baseline will be followed up according to the same tumor assessment schedule;
²⁴ Clinical assessment of superficial disease must be carried out on the same date as the imaging studies and will include photographs of all superficial metastasis lesions. All lesions must be recorded in the eCRF;
²⁵ First HER2 inhibitor dose will be administered intravenously over 90 minutes (±15 minutes). If no infusion related reaction occurs after first dose, subsequent doses can be administered intravenously over 60 minutes (±15 minutes);
²⁶ Palbociclib will be administered at 125 mg/day orally for 3 weeks followed by 1 week off. Patients are required to complete patient diary on Day 1 of each cycle for drug accountability and return all bottles of palbociclib;
²⁷ Examination or test results should be obtained before study drug administration;
²⁸ Examination or test results should be obtained within 7 days prior to first trial treatment;
²⁹ Only be performed when clinically significant abnormal findings are found at EOT visit;

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A medicinal product comprising:
a HER2 inhibitor and a CDK inhibitor, wherein said HER2 inhibitor is capable of binding to a first HER2 antigen and a second HER2 antigen, and wherein said CDK inhibitor inhibits CDK4 and/or CDK6.

2. (canceled)

3. (canceled)

4. (canceled)

5. (canceled)

6. (canceled)

7. The medicinal product according to claim 1, wherein said HER2 inhibitor is a bispecific antibody or an antigen binding portion thereof.

8. (canceled)

9. (canceled)

10. (canceled)

11. (canceled)

12. The medicinal product according to claim 1, wherein variable region of first light chain and/or second light chain of HER2 inhibitor comprises an amino acid sequence as set forth in any one of SEQ ID NO: 1-6.

13. (canceled)

14. (canceled)

15. The medicinal product according to claim 1, wherein first light chain of HER2 inhibitor comprises an amino acid sequence as set forth in any one of SEQ ID NO: 7-12, and/or, second light chain of HER2 inhibitor comprises an amino acid sequence as set forth in any one of SEQ ID NO: 7-12.

16. (canceled)

17. The medicinal product according to claim 1, wherein the variable region of first heavy chain of HER2 inhibitor comprises an amino acid sequence as set forth in SEQ ID NO: 13; and variable region of second heavy chain of HER2 inhibitor comprises an amino acid sequence as set forth in SEQ ID NO: 14

18. (canceled)

19. (canceled)

20. The medicinal product according to claim 1, wherein two heavy chains of HER2 inhibitor thereof comprise a sequence as set forth in any one of SEQ ID NO: 15-18.

21. (canceled)

22. The medicinal product according to claim 1, wherein said CDK inhibitor has a structure of formula I: 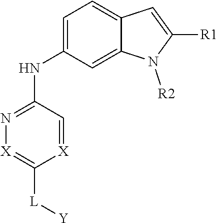
wherein X is CR⁹, or N;
R¹ is C₁₋₈alkyl, CN, C(O)OR⁴ or CONR⁵R⁶, a 5-14 membered heteroaryl group, or a 3-14 membered cycloheteroalkyl group;
R² is C₁₋₈alkyl, C₃₋ᵢ₄cycloalkyl, or a 5-14 membered heteroaryl group, and wherein R² may be substituted with one or more C₁₋₈alkyl, or OH;
L is a bond, C₁₋₈alkylene, C(O), or C(O)NR¹⁰, and wherein L may be substituted or unsubstituted; Y is H, R¹¹, NR¹²R¹³, OH, or Y is part of the following group 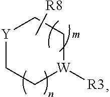
where Y is CR⁹ or N; where 0-3 R⁸ may be present, and R⁸ is C₁₋₈alkyl, oxo, halogen, or two or more R⁸ may form a bridged alkyl group; W is CR⁹, or N;
R³ is H, C₁₋₈alkyl, C₁₋₈alkylR¹⁴, C₃-i₄cycloalkyl, C(O)C₁₋₈ alkyl, C₁₋₈haloalkyl, C₁₋₈alkylOH, C(O)NR¹⁴R¹⁵, Ci-gcyanoalkyl, C(O)R¹⁴, Co-₈alkylC(O)C₀₋₈alkylNR¹⁴R¹⁵, C₀₋₈alkylC(O)OR¹⁴, NR¹⁴R¹⁵, SO₂C₁₋₈alkyl, C₁₋₈alkylC₃-i₄cycloalkyl, C(O)C₁₋₈alkylC₃-i₄cycloalkyl, C₁₋₈alkoxy, or OH which may be substituted or unsubstituted when R³ is not H, R⁹ is H or halogen; R⁴, R⁵, R⁶, R⁷, R¹⁰, R¹¹, R¹², R¹³, R¹⁴, and R¹⁵ are each independently selected from H, C₁₋₈alkyl, C₃—H cycloalkyl, a 3-14 membered cycloheteroalkyl group, a Cβ-α aryl group, a 5-14 membered heteroaryl group, alkoxy, C(O)H, C(N)OH, C(N)OCH₃, C(O)C₁₋₃alkyl, C₁₋₈alkylNH₂, C₁₋₆ alkylOH, and wherein R⁴, R⁵, R⁶, R⁷, R¹⁰, R¹¹, R¹², and R¹³, R¹⁴, and R¹⁵ when not H may be substituted or unsubstituted; m and n are independently 0-2; and wherein L, R³, R⁴ R⁵, R⁶, R⁷, R¹⁰, R¹¹, R¹², and R¹³, R¹⁴, and R¹⁵ may be substituted with one or more of C₁₋₈alkyl, C₂₋₈alkenyl, C₂₋₈alkynyl, C₃₋₁₄cycloalkyl, 5-14 membered heteroaryl group, C₆₋₁₄aryl group, a 3-14 membered cycloheteroalkyl group, OH, (O), CN, alkoxy, halogen, Or NH₂.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. The medicinal product according to claim 1, wherein said CDK inhibitor has a structure of formula II: 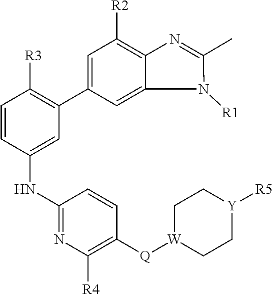
R1 is C₃-C₅ alkyl, C₃-C₅ cycloalkyl or cyclopropyl-methyl; R2 and R3 are H or fluorine, wherein at least one of R2 or R3 is fluorine; R4 is H or CH₃;
R5 is Ci-C₆ alkyl or —NR6R7 wherein R6 and R7 are C1-C3 alkyl; Q is CH₂, O, S or a direct bond; and
W and Y are C or N, wherein at least one of W or Y is N and wherein when Q is O or S, W is C;
or a pharmaceutically acceptable salt thereof.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. The medicinal product according to claim 1, wherein said CDK inhibitor has a structure of formula III, 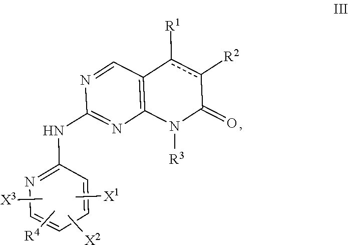
X¹, X², and X³ are independently hydrogen, halogen, C₁-C₆ alkyl, C₁-C₆ halo alkyl, C₁-C₈ alkoxy, C₁-C₈ alkoxyalkyl, CN, NO₂, OR⁵, NR⁵R⁶, CO₂R⁵, COR⁵, S(O)ₙR⁵, CONR⁵R⁶, NR⁵COR⁶, NR⁵SO₂R⁶, SO₂NR⁵R⁶, and P(O)(OR⁵)(OR⁶); with the proviso that at least one of X¹, X², and X³ must be hydrogen;
n is selected from 0, 1 and 2;
R¹ is, in each instance, independently, hydrogen, halogen, C₁-C₆ alkyl, C₁-C₆ halo alkyl, C₁-C₆ hydroxyalkyl, or C₃-C₇ cycloalkyl;
R² and R⁴ are each independently selected from hydrogen, halogen, C₁-C₈ alkyl, C₃-C₇ cycloalkyl, C₁-C₈ alkoxy, C₁-C₈ alkoxyalkyl, C₁-C₈ halo alkyl, C₁-C₈ hydroxyalkyl, C₂-C₈ alkenyl, C₂-C₈ alkynyl, nitrile, nitro, OR⁵, SR⁵, NR⁵R⁶, N(O)R⁵R⁶, P(O)(OR⁵)(OR⁶), (CR⁵R⁶)ₘNR⁷R⁸, COR⁵, (CR⁴R⁵)ₘC(O)R⁷, CO₂R⁵, CONR⁵R⁶, C(O)NR⁵SO₂R, NR⁵SO₂R⁶, C(O)NR⁵OR⁶, S(O)ₙR⁵, SO₂NR⁵R⁶, P(O)(OR⁵)(OR⁶), (CR⁵R⁶)ₘP(O)(OR⁷)(OR⁸), (CR⁵R⁶)ₘ-aryl, (CR⁵R⁶)ₘ-heteroaryl, T(CH₂)ₘQR⁵, —C(O)T(CH₂)ₘQR⁵, NR⁵C(O)T(CH₂)ₘQR⁵, and —CR⁵═CR⁶C(O)R⁷; or
R¹ and R² may form a carbocyclic group containing 3-7 ring members, preferably 5-6 ring members, up to four of which can optionally be replaced with a heteroatom independently selected from oxygen, sulfur, and nitrogen, and wherein the carbocyclic group is unsubstituted or substituted with one, two, or three groups independently selected from halogen, hydroxy, hydroxyalkyl, nitrile, lower C₁-C₈ alkyl, lower C₁-C₈ alkyl, alkoxycarbonyl, alkylcarbonyl, alkylcarbonylamino, aminoalkyl, trifluoromethyl, N-hydroxyacetamide, trifluoromethylalkyl, amino, and mono or dialkylamino, (CH₂)ₘC(O)NR⁵R⁶, and O(CH₂)ₘC(O)OR⁵, provided, however, that there is at least one carbon atom in the carbocyclic ring and that if there are two or more ring oxygen atoms, the ring oxygen atoms are not adjacent to one another;
T is O, S, NR⁷, N(O)R⁷, NR⁷R⁸W, or CR⁷R⁸;
Q is O, S, NR⁷, N(O)R⁷, NR⁷R⁸W, CO₂, O(CH₂)ₘ-heteroaryl, O(CH₂)ₘS(O)ₙR⁸, (CH₂)-heteroaryl, or a carbocyclic group containing from 3-7 ring members, up to four of which ring members are optionally heteroatoms independently selected from oxygen, sulfur, and nitrogen, provided, however, that there is at least one carbon atom in the carbocyclic ring and that if there are two or more ring oxygen atoms, the ring oxygen atoms are not adjacent to one another, wherein the carbocyclic group is unsubstituted or substituted with one, two, or three groups independently selected from alkoxycarbonyl, alkylcarbonyl, alkylcarbonylamino, aminoalkyl, trifluoromethyl, N-hydroxyacetamide, trifluoromethylalkyl, amino, and mono or dialkylamino;
W is an anion selected from the group consisting of chloride, bromide, trifluoroacetate, and triethylammonium;
m is selected from 0, 1, 2, 3, 4, 5 and 6;
R⁴ and one of X¹, X² and X³ may form an aromatic ring containing up to three heteroatoms independently selected from oxygen, sulfur, and nitrogen, and optionally substituted by up to 4 groups independently selected from alogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, mono- or dialkylamino, N-hydroxyacetamido, aryl, heteroaryl, carboxyalkyl, nitrile, NR⁷SO₂R⁸, C(O)NR⁷R⁸, NR⁷C(O)R⁸, C(O)OR⁷, C(O)NR⁷SO₂R⁸, (CH₂)ₘS(O)ₙR⁷, (CH₂)ₘ-heteroaryl, O(CH₂)ₘ-heteroaryl, (CH₂), C(O)NR⁷R⁸, O(CH₂)ₘC(O)OR⁷, (CH₂)ₘSO₂NR⁷R⁸, and C(O)R⁷;
R³ is hydrogen, aryl, C₁-C₈ alkyl, C₁-C₈ alkyl, C₃-C₇ cycloalkyl, or C₃-C₇-heterocyclyl;
R⁵ and R⁶ are each independently hydrogen, C₁-C₈ alkyl, C₂-C₅ alkenyl, C₂-C₈ alkynyl, arylalkyl, cycloalkyl, heterocycloalkyl, aryl, heteroaryl, or heterarylalkyl; or
R⁵ and R⁶, when attached to the same nitrogen atom, taken together with the nitrogen to which they are attached, form a heterocyclic ring containing from 3-8 ring members, up to four of which members can optionally be replaced with heteroatoms independently selected from oxygen, sulfur, S(O), S(O)₂, and nitrogen, provided, however, that there is at least one carbon atom in the heterocyclic ring and that if there are two or more ring oxygen atoms, the ring oxygen atoms are not adjacent to one another, wherein the heterocyclic group is unsubstituted or substituted with one, two or three groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, nitrile, mono- or dialkylamino, N-hydroxyacetamido, aryl, heteroaryl, carboxyalkyl, NR⁷SO₂R⁸, C(O)NR⁷R⁸, NR⁷C(O)R⁸, C(O)OR⁷, C(O)NR⁷SO₂R⁸, (CH₂)ₘS(O)ₙR⁷, (CH₂)ₘ-heteroaryl, O(CH₂)ₘ-heteroaryl, (CH₂)ₘC(O)NR⁷R⁸, O(CH₂)ₘC(O)OR⁷, and (CH₂)SO₂NR⁷R⁸;
R⁷ and R⁸ are, each independently, hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl; or
R⁷ and R⁸, when attached to the same nitrogen atom, taken together with the nitrogen to which they are attached, may form a heterocyclic ring containing from 3-8 ring members, up to four of which members are optionally heteroatoms independently selected from oxygen, sulfur, S(O), S(O)₂, and nitrogen, provided, however, that there is at least one carbon atom in the heterocyclic ring and that if there are two or more ring oxygen atoms, the ring oxygen atoms are not adjacent to one another, wherein the heterocyclic group is unsubstituted or substituted with one, two or three groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, nitrile, mono- or dialkylamino, N-hydroxyacetamido, aryl, heteroaryl, carboxyalkyl; and the pharmaceutically acceptable salts, esters, amides, and prodrugs thereof.

49. The medicinal product according to claim 1, wherein said CDK inhibitor has a structure of formula IV: 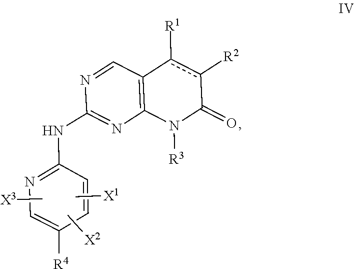
wherein X¹, X², and X³ are independently hydrogen, halogen, C₁-C₆ alkyl, C₁-C₆ halo alkyl, C₁-C₈ alkoxy, C₁-C₈ alkoxyalkyl, CN, NO₂, OR⁵, NR⁵R⁶, CO₂R⁵, COR⁵, S(O)ₙR⁵, CONR⁵R⁶, NR⁵COR⁶, NR⁵SO₂R⁶, SO₂NR⁵R⁶, and P(O)(OR⁵)(OR⁶); with the proviso that at least one of X¹, X², and X³ must be hydrogen;
n is selected from 0, 1 and 2;
R¹ is, in each instance, independently, hydrogen, halogen, C₁-C₆ alkyl, C₁-C₆ halo alkyl, C₁-C₆ hydroxyalkyl, or C₃-C₇ cycloalkyl;
R² and R⁴ are each independently selected from hydrogen, halogen, C₁-C₈ alkyl, C₃-C₇ cycloalkyl, C₁-C₈ alkoxy, C₁-C₈ alkoxyalkyl, C₁-C₈ halo alkyl, C₁-C₈ hydroxyalkyl, C₂-C₈ alkenyl, C₂-C₈ alkynyl, nitrile, nitro, OR⁵, SR⁵, NR⁵R⁶, N(O)R⁵R⁶, P(O)(OR⁵)(OR⁶), (CR⁵R⁶)ₘNR⁷R⁸, COR⁵, (CR⁴R⁵)ₘC(O)R⁷, CO₂R5, CONR⁵R⁶, C(O)NR⁵SO₂R, NR⁵SO₂R⁶, C(O)NR⁵OR⁶, S(O)ₙR⁵, SO₂NR⁵R⁶, P(O)(OR⁵)(OR⁶), (CR⁵R⁶)ₘP(O)(OR⁷)(OR⁸), (CR⁵R⁶)ₘ-aryl, (CR⁵R⁶)ₘ-heteroaryl, T(CH₂)ₘQR⁵, —C(O)T(CH₂)ₘQR⁵, NR⁵C(O)T(CH₂)ₘQR⁵, and —CR⁵═CR⁶C(O)R⁷; or
R¹ and R² may form a carbocyclic group containing 3-7 ring members, preferably 5-6 ring members, up to four of which can optionally be replaced with a heteroatom independently selected from oxygen, sulfur, and nitrogen, and wherein the carbocyclic group is unsubstituted or substituted with one, two, or three groups independently selected from halogen, hydroxy, hydroxyalkyl, nitrile, lower C₁-C₈ alkyl, lower C₁-C₈ alkyl, alkoxycarbonyl, alkylcarbonyl, alkylcarbonylamino, aminoalkyl, trifluoromethyl, N-hydroxyacetamide, trifluoromethylalkyl, amino, and mono or dialkylamino, (CH₂)ₘC(O)NR⁵R⁶, and O(CH₂)ₘC(O)OR⁵, provided, however, that there is at least one carbon atom in the carbocyclic ring and that if there are two or more ring oxygen atoms, the ring oxygen atoms are not adjacent to one another;
T is O, S, NR⁷, N(O)R⁷, NR⁷R⁸W, or CR⁷R⁸;
Q is O, S, NR7, N(O)R⁷, NR⁷R⁸W, CO₂, O(CH₂)ₘ-heteroaryl, O(CH₂)ₘS(O)ₙR⁸, (CH₂)-heteroaryl, or a carbocyclic group containing from 3-7 ring members, up to four of which ring members are optionally heteroatoms independently selected from oxygen, sulfur, and nitrogen, provided, however, that there is at least one carbon atom in the carbocyclic ring and that if there are two or more ring oxygen atoms, the ring oxygen atoms are not adjacent to one another, wherein the carbocyclic group is unsubstituted or substituted with one, two, or three groups independently selected from alkoxycarbonyl, alkylcarbonyl, alkylcarbonylamino, aminoalkyl, trifluoromethyl, N-hydroxyacetamide, trifluoromethylalkyl, amino, and mono or dialkylamino;
W is an anion selected from the group consisting of chloride, bromide, trifluoroacetate, and triethylammonium;
m is selected from 0, 1, 2, 3, 4, 5 and 6;
R⁴ and one of X¹, X² and X³ may form an aromatic ring containing up to three heteroatoms independently selected from oxygen, sulfur, and nitrogen, and optionally substituted by up to 4 groups independently selected from alogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, mono- or dialkylamino, N-hydroxyacetamido, aryl, heteroaryl, carboxyalkyl, nitrile, NR⁷SO₂R⁸, C(O)NR⁷R⁸, NR⁷C(O)R⁸, C(O)OR⁷, C(O)NR⁷SO₂R⁸, (CH₂)ₘS(O)ₙR⁷, (CH₂)ₘ-heteroaryl, O(CH₂)ₘ-heteroaryl, (CH₂), C(O)NR⁷R⁸, O(CH₂)ₘC(O)OR⁷, (CH₂)ₘSO₂NR⁷R⁸, and C(O)R⁷;
R³ is hydrogen, aryl, C₁-C₈ alkyl, C₁-C₈ alkyl, C₃-C₇ cycloalkyl, or C₃-C₇-heterocyclyl;
R⁵ and R⁶ are each independently hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl; or
R⁵ and R⁶, when attached to the same nitrogen atom, taken together with the nitrogen to which they are attached, form a heterocyclic ring containing from 3-8 ring members, up to four of which members can optionally be replaced with heteroatoms independently selected from oxygen, sulfur, S(O), S(O)₂, and nitrogen, provided, however, that there is at least one carbon atom in the heterocyclic ring and that if there are two or more ring oxygen atoms, the ring oxygen atoms are not adjacent to one another, wherein the heterocyclic group is unsubstituted or substituted with one, two or three groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, nitrile, mono- or dialkylamino, N-hydroxyacetamido, aryl, heteroaryl, carboxyalkyl, NR⁷SO₂R⁸, C(O)NR⁷R⁸, NR⁷C(O)R⁸, C(O)OR⁷, C(O)NR⁷SO₂R⁸, (CH₂)ₘS(O)ₙR⁷, (CH₂)ₘ-heteroaryl, O(CH₂)ₘ-heteroaryl, (CH₂)ₘC(O)NR⁷R⁸, O(CH₂)ₘC(O)OR⁷, and (CH₂)SO₂NR⁷R⁸;
R⁷ and R⁸ are, each independently, hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl; or
R⁷ and R⁸, when attached to the same nitrogen atom, taken together with the nitrogen to which they are attached, may form a heterocyclic ring containing from 3-8 ring members, up to four of which members are optionally heteroatoms independently selected from oxygen, sulfur, S(O), S(O)₂, and nitrogen, provided, however, that there is at least one carbon atom in the heterocyclic ring and that if there are two or more ring oxygen atoms, the ring oxygen atoms are not adjacent to one another, wherein the heterocyclic group is unsubstituted or substituted with one, two or three groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, nitrile, mono- or dialkylamino, N-hydroxyacetamido, aryl, heteroaryl, carboxyalkyl; and the pharmaceutically acceptable salts, esters, amides, and prodrugs thereof.

50. (canceled)

51. (canceled)

52. (canceled)

53. (canceled)

54. (canceled)

55. (canceled)

56. (canceled)

57. The medicinal product according to claim 1, wherein said medicinal product further comprises a fulvestrant.

58. (canceled)

59. (canceled)

60. (canceled)

61. (canceled)

62. A method of preventing, alleviating or treating tumor or inhibiting tumor growth in a subject in need thereof, comprising: administrating said HER2 inhibitor in combination with said CDK inhibitor of claim 1 to the subject.

63. A method of preventing, alleviating or treating tumor or inhibiting tumor growth in a subject in need thereof, comprising: administrating said composition of claim 1 to the subject.

64. (canceled)

65. The method according to claim 62, wherein said tumor comprises metastatic tumor, early tumor and/or locally advanced tumor.

66. The method according to claim 62, wherein said tumor comprises HER2 positive tumor and/or HER2 low-expression tumor.

67. The method according to claim 62, wherein said tumor comprises a breast cancer and/or a gastric cancer.

68. (canceled)

69. (canceled)

70. (canceled)

71. (canceled)

72. (canceled)

73. The method according to claim 62, wherein said HER2 inhibitor is administrated to the subject in need at a dose of about 1 mg/kg to about 30 mg/kg.

74. (canceled)

75. (canceled)

76. The method according to claim 62, wherein said CDK inhibitor is administrated to the subject in need at a dose of about 10 mg/kg to about 50 mg/kg.

77. (canceled)

78. The method according to claim 62, wherein said method further comprises administering to said subject an effective amount of fulvestrant.

79. (canceled)

80. The method according to claim 78, wherein fulvestrant in said medicinal product is administrated to the subject in need at a dose of about 400 mg to about 600 mg.

81. (canceled)

82. (canceled)

83. (canceled)

84. (canceled)
